(19) United States
(12) Patent Application Publication
(10) Pub. No.: US 2024/0318167 A1
(43) Pub. Date: Sep. 26, 2024

(54) HUMANIZED CELL LINE

(71) Applicants: The University of Tokyo, Tokyo (JP); Wisconsin Alumni Research Foundation (WARF), Madison, WI (US)

(72) Inventors: Yoshihiro Kawaoka, Middleton, WI (US); Kosuke Takada, Tokyo (JP); Masaki Imai, Kawasaki-shi (JP)

(21) Appl. No.: 18/525,460

(22) Filed: Nov. 30, 2023

Related U.S. Application Data
(63) Continuation of application No. 16/785,449, filed on Feb. 7, 2020, now Pat. No. 11,851,648.
(60) Provisional application No. 62/803,266, filed on Feb. 8, 2019.

(51) Int. Cl. C12N 15/10; C12N 5/071; C12N 9/10; C12Q 1/04

(52) U.S. Cl. CPC C12N 15/102; C12N 5/0602; C12N 9/10; C12Q 1/04; C12Y 204/99001

(57) ABSTRACT

A mammalian or avian cell line that expresses high levels of human influenza virus receptors is provided. In one embodiment, the cell line supports human influenza virus, e.g., human A/H3 influenza virus, isolation and growth much more effectively than corresponding conventional (unmodified) cells or in corresponding human virus receptor-overexpressing cells, and the propagated viruses may maintain higher genetic stability than in the corresponding cells.

### CROSS-REFERENCE TO RELATED APPLICATIONS

This application is a continuation of U.S. application Ser. No. 16/785,449, filed Feb. 7, 2020, which claims the benefit of the filing date of U.S. application No. 62/803,266, filed on Feb. 8, 2019, the disclosures of which are incorporated by reference herein in their entirety.

### STATEMENT OF GOVERNMENT RIGHTS

This invention was made with government support under HHSN272201400008C awarded by the National Institutes of Health. The government has certain rights in the invention.

### INCORPORATION BY REFERENCE OF SEQUENCE LISTING

This application contains a sequence listing. It has been submitted electronically as an XML file titled “800105US2.xml.” The sequence listing is 135,074 bytes in size and was created on Jun. 6, 2024. It is hereby incorporated by reference in its entirety.

### BACKGROUND

The influenza A and B viruses possess two major surface glycoproteins, hemagglutinin (HA) and neuraminidase (NA). HA recognizes sialic acid-containing receptors on the cell surface, while NA cleaves sialic acids from receptors on cellular surfaces to facilitate the release of progeny virions from the surface of infected cells (Gamblin and Skehel, 2010). HA is also the major antigen stimulating the host's protective immunity, specifically the production of neutralizing antibodies.

Virus isolation from clinical specimens is an essential tool for the identification and characterization of circulating viruses. Currently, two subtypes of influenza A viruses (A/H1N1 and A/H3N2) and two lineages of influenza B viruses (B/Yamagata- and B/Victoria-lineage) are cocirculating in the human population and cause epidemics of seasonal influenza. Madin-Darby canine kidney (MDCK) cells are the most widely used cell line for isolation and propagation of human influenza viruses. This cell line shows high susceptibility to influenza viruses; however, it supports the growth of recent A/H3N2 viruses poorly. Furthermore, passaging of influenza viruses in MDCK cells often leads to the selection of variants with mutations in their HA and/or NA genes (Chambers et al., 2014; Lee et al., 2013; Tamura et al., 2013; Lin et al., 2017; Li et al., 2009; Oh et al., 2008). The emergence of such variants carrying mutations relevant to adaptation of influenza viruses to cell culture could distort the evaluation of the antigenic, genetic, and antiviral properties of circulating influenza viruses. For example, the emergence of mutations that confer receptor-binding activity to the NA of A/H3N2 viruses, such as the aspartic acid-to-glycine substitution at position 151 (D151G) (Moh et al., 2015; Lin et al., 2010; Zhu et al., 2012), is problematic for characterization of HA antigenicity by means of hemagglutination-inhibition, virus-neutralization, and focus reduction assays because the receptor-binding activity of NA contributes to the results of these assays. Nevertheless, many laboratories use MDCK cells to isolate A/H3N2 viruses A GISAID EpiFlu database analysis by Lee et al. (2013) showed that approximately 30% of MDCK-cultured A/H3N2 isolates possess an amino acid change at position 151. Therefore, currently circulating A/H3N2 strains should be isolated and propagated in cell lines that can faithfully maintain their characteristics.

The HAs of human influenza viruses prefer to bind to glycans that end with sialic acid linked to galactose by α2,6-linkages, whereas avian virus HAs preferentially bind to glycans that terminate with sialic acid linked to galactose by α2,3-linkages (Connor et al., 1994; Rogers and Paulson, 1983; Stevens et al., 2006). Correspondingly, epithelial cells in the human upper respiratory tract express predominantly α2,6-sialoglycans (van Riel et al., 2006; Shinya et al., 2006). Although MDCK cells expressing both α2,6- and α2,3-sialoglycans are suitable for the isolation of influenza viruses from multiple animal species, this cell line has been shown to express relatively low levels of α2,6-sialoglycans (Lin et al., 2017; Hatakeyama et al., 2005; Matrosovich et al., 2003). Previously, our group and others engineered MDCK cells to overexpress α2,6-sialoglycans (Hatakeyama et al., 2005; Matrosovich et al., 2003). These modified MDCK cells (designated AX4 or MDCK-SIAT1) displayed a higher sensitivity for human influenza virus isolation than a conventional MDCK cell line (Oh et al., 2008; Hatakeyama et al., 2005), yet they still expressed α2,3-sialoglycans. Importantly, as with conventional MDCK cells, variants with mutations in either HA or NA have been detected when seasonal influenza viruses were passaged through MDCK-SIAT1 cells (Tamura et al., 2013; Li et al., 2009). Therefore, an alternative cell line that supports efficient isolation and propagation of human influenza viruses without any cell culture-adaptive mutations is necessary for accurate characterization of circulating viruses and possibly for efficient vaccine production in cells.

### SUMMARY

The present disclosure relates to a mammalian or avian cell line that is genetically modified to support, for example, more efficient isolation and/or amplification (propagation) of human influenza viruses, and in particular human H3 influenza viruses. The disclosed cell lines may be genetically modified to decrease expression of alpha-2,3-linked sialic acids on the cell surface and to increase expression of alpha-2,6-linked sialic acids relative to a parental cell lines that are not modified to alter expression of alpha-2,3-linked sialic acids, alpha-2,6-linked sialic acids, or both. In one embodiment, the modified mammalian or avian cell lines are modified to express high levels of human influenza virus receptors and low levels of avian inlfuenza virus receptors. In one embodiment, the cell line is a mammalian cell line, e.g., a non-human cell line such as a primate cell line, or a canine cell line. In one embodiment, the modified cell line is a modified MDCK cell line that has decreased expression of alpha-2,3-linked sialic acid relative to AX-4, or increased expression of alpha-2,6-linked sialic acid relative to unmodified MDCK cells. In one embodiment, the modified cell line is hCK, which supports more efficient isolation and amplification of human influenza viruses compared to MDCK and AX-4 cells. In one embodiment, the decrease in expression of alpha-2,3-linked sialic acids is due to a genetic modification that decreases or eliminates expression of one or more sialyltransferases that produce alpha-2,3-linked sialic acids, a genetic modification including but not limited to an insertion of one or more nucleotides, a deletion of one or more nucleotides, a substitution of one or more nucleotides, or any combination thereof, in one or more sialyltransferase genes. In one embodiment, the genetic modification includes an insertion of one or more nucleotides in one or more sialyltransferase genes. In one embodiment, the genetic modification includes a deletion of one or more nucleotides in one or more sialyltransferase genes. In one embodiment, the genetic modification includes a substitution of one or more nucleotides in one or more sialyltransferase genes. In one embodiment, the genetic modification includes an insertion of one or more nucleotides in at least one sialyltransferase gene In one embodiment, the genetic modification includes a deletion of one or more nucleotides in at least one sialyltransferase gene. In one embodiment, the genetic modification includes a substitution of one or more nucleotides in at least one sialyltransferase gene. The genetic modifications that decrease expression of alpha-2,3-linked sialic acids may be the result of any method that “knocks down” or “knocks out” expression, methods including the uses of recombinase systems such as CRISPR/Cas, TALEN or zinc finger binding proteins. In one embodiment, the increase in expression of alpha-2,6-linked sialic acids is due to a genetic modification that increase expression of one or more sialyltransferases that produce alpha-2,6-linked sialic acids, a genetic modification including but not limited to an expression cassette comprising a nucleotide sequence encoding a sialyltransferase that that produces alpha-2,6-linked sialic acids, e.g., a human β-galactoside α2,6-sialyltransferase I (ST6Gal I) gene.

In one embodiment, an isolated recombinant mammalian or avian cell comprising a reduced amount of cell surface β-galactoside α2,3 sialyl residues and an increased amount of human β-galactoside α2,6 sialyl residues relative to a corresponding non-recombinant mammalian or avian cell is provided. In one embodiment, the isolated recombinant cell is a non-human mammalian cell. In one embodiment, the isolated recombinant cell is a canine or non-human primate cell. In one embodiment, the reduced amount of surface β-galactoside α2,3 sialyl residues is the result of a reduced amount or activity of one or more α2,3 sialyltransferases, e.g., a reduction in the amount or activity of one or more α2,3 sialyltransferases of at least 5%, 10%, 20%, 50%, 70%, 80%, 90%, 95% or more, which may result in a reduction of at least 5%, 10%, 20%, 50%, 70%, 80%, 90%, 95% or more in α2,3 sialyl residues, in the recombinant cell. In one embodiment, the amount or activity of α2,3 sialyltransferases, or the amount of α2,3 sialyl residues, in the recombinant cell is undetectable. In one embodiment, the α2,3 sialyltransferase gene that is modified encodes an α2,3 sialyltransferase that has at least 80%, 85%, 87%, 90%, 92%, 95%, 96%, 97%, 98%, or 99% amino acid sequence identity to any one of SEQ ID Nos. 6, 8, 10, 12, 14, 16 or 18, or a nucleotide sequence having at least 80%, 85%, 87%, 90%, 92%, 95%, 96%, 97%, 98%, or 99% nucleotide acid sequence identity to any one of SEQ ID Nos. 5, 7, 9, 11, 13, 15, or 17. In one embodiment, the isolated recombinant cell comprises an expression cassette encoding human β-galactoside α2,6 sialyltransferase I (ST6Gal-I) or ST6Gal-II. In one embodiment, the ST6Gal-I or ST6Gal-II comprises a protein having at least 80% amino acid sequence identity to any one of SEQ ID Nos. 1-4, 101 or 150. In one embodiment, the α2,6 sialyltransferase gene encodes an α2,6 sialyltransferase that has at least 85%, 87%, 90%, 92%, 95%, 96%, 97%, 98%, or 99% amino acid sequence identity to any one of SEQ ID Nos. 1-4, 101 or 150, or a nucleotide sequence having at least 80%, 85%, 87%, 90%, 92%, 95%, 96%, 97%, 98%, or 99% nucleotide acid sequence identity to SEQ ID Nos. 101 or 151 In one embodiment, the human β-galactoside 2,6 sialyltransferase amount or activity in the recombinant cell in creased by at least 1%, 5%, 10%, 20%, 50%, 70%, 80%, 90%, 95% or more. In one embodiment, one or more β-galactoside α2,3 sialyltransferase genes are mutated so as to reduce the amount of cell surface β-galactoside α2,3 sialyl residues. In one embodiment, two or more of ST3Gal-I, ST3Gal-II, ST3Gal-III, ST3Gal-IV, ST3Gal-V. ST3Gal-VI, or ST3Gal-II-like genes are mutated. In one embodiment, three, four, five, six or seven of ST3Gal-1. ST3Gal-II, ST3Gal-III, ST3Gal-IV, ST3Gal-V, ST3Gal-VI, or ST3Gal-II-like genes are mutated. In one embodiment, the ST3 genes have at least 80% nucleic acid sequence identity to any one of SEQ ID Nos. 5, 7, 9, 11, 13, 15, or 17. In one embodiment, the reduction in cell surface β-galactoside α2,3 sialyl residues is the result of reduced expression of one or more ST3 sialyltransferases. In one embodiment, the one or more ST3 sialyltransferases have at least 80% amino acid sequence identity to any one of SEQ ID Nos. 6, 8, 10, 12, 14, 16, or 18. In one embodiment, influenza H3 viruses replicate more efficiently in the recombinant cell relative to the non-recombinant cell.

Further provided is an isolated recombinant mammalian or avian cell, comprising a reduced amount of cell surface β-galactoside α2,3 sialyl residues relative to a corresponding non-recombinant mammalian or avian cell. In one embodiment, one or more of ST3Gal-I, ST3Gal-II, ST3Gal-III, ST3Gal-IV, ST3Gal-V, ST3Gal-VI, or ST3Gal-II-like genes in the recombinant cell are mutated. In one embodiment, a combination of ST3Gal-1. ST3Gal-II, ST3Gal-III, ST3Gal-IV, ST3Gal-V, ST3Gal-VI, or ST3Gal-II-like genes in the recombinant cell are mutated. In one embodiment, ST3Gal-1. ST3Gal-II, ST3Gal-III. ST3Gal-IV, ST3Gal-V, ST3Gal-VI, and ST3Gal-II-like genes in the recombinant cell are mutated.

The recombinant cells described herein are useful, for example, in virus isolation, vaccine production and in diagnostics. For example, the recombinant cells allow for isolation and/or amplification of progeny viruses. Moreover, HA assays generally are not used to detect human H3N2 viruses. The recombinant cells may be advantageous in that regard, e.g., to amplify virus.

Further, recombinant cells that have increased β-galactoside α2,6 sialyl residues can be used as a source of isolated β-galactoside α2,6 sialyl, which in turn may be used to coat surfaces such as beads, to inhibit galectin(s), to isolate or detect Sambucus nigra agglutinin (SNA), Sambucus sieboldiana (SSA) or Trichosanthes japonica agglutinin I (TJA-I).

In one embodiment, a method of modifying the amount of cell surface β-galactoside α2,3 sialyl residues and human β-galactoside α2,6 sialyl residues on a mammalian or an avian cell is provided. In one embodiment, the method includes mutating one or more β-galactoside α2,3 sialyltransferase (ST3Gal) genes, and overexpressing a human β-galactoside α2,6 sialyltransferase (ST6Gal) gene, in a parental mammalian or avian cell so as to result in a modified mammalian or avian cell having a reduced amount of cell surface β-galactoside α2,3 sialyl residues and an increased amount of human β-galactoside α2,6 sialyl residues on the surface of the modified cell relative to the corresponding parental cell. In one embodiment, the one or more ST3Gal genes are mutated using a genome editing system, e.g., a CRISPR/Cas9, Zinc Finger Nuclease (ZFN) or transcription activator-like effector nuclease (TALEN) system. In one embodiment, the mutations include one or more nucleotide insertions or one or more nucleotide deletions, or both, in one or more ST3 genes. In one embodiment, the modified cell comprises an expression cassette comprising a ST6Gal open reading frame. In one embodiment, the modified cell is a kidney cell. In one embodiment, the modified cell is a canine cell.

Methods of using the recombinant cell include a method of propagating an influenza virus, e.g., a human influenza virus, for vaccine production. In one embodiment, the influenza virus is an influenza A virus. In one embodiment, the influenza virus is an influenza B virus. In one embodiment, the influenza virus is a H3 virus. In one embodiment, the virus is A/H1N1, A/H3N2, a B/Yamagata-lineage influenza B virus or a B/Victoria-lineage influenza B virus.

A further method which employs the recombinant cell is a method of isolating an influenza virus. The method includes providing a sample from an avian or a mammal suspected of being infected with an influenza virus; and contacting the recombinant cell with the sample. In one embodiment, the method further includes determining whether the sample is infected with an influenza virus. In one embodiment, the method further includes identifying the HA and/or NA subtype of the virus.

In one embodiment, the cell line is a modified MDCK cell line, ‘hCK’ for ‘humanized MDCK’ cells, which was prepared using CRISPR/Cas-mediated gene knock-out methods to down-regulate sialyltransferases that catalyze the synthesis of alpha-2,3-linked sialic acids, and overexpression of a sialyltransferase that catalyzes the synthesis of alpha-2,6-linked sialic acids. hCK cells express low levels of alpha-2,3-linked sialic acids and high levels of alpha-2,6-linked sialic acids (similar to human epithelial cells in the upper respiratory tract). As disclosed herein, hCK cells allow for the isolation of H3N2 human influenza viruses 10-100 better than the AX-4 cell line. Efficient isolation and amplification of influenza viruses including human influenza viruses is advantageous for vaccine production (possibly supporting better replication), e.g., for seasonal influenza virus vaccine production, as seasonal human influenza viruses often replicate inefficiently in unmodified MDCK cells and even in MDCK (AX-4) cells overexpressing alpha-2,6-linked sialic acids on their surface, to which human influenza viruses bind efficiently. In one embodiment, the titer of human influenza viruses on the modified cell line disclosed herein is at least one log, at least two logs, at least three logs or greater than in unmodified MDCK cells, MDCK (AX-4) cells or MDCK-SIAT1 cells (Li et al., 2009).

### BRIEF DESCRIPTION OF THE FIGURES

FIGS. 1A-1P. Sensitivity of hCK cells to human influenza virus growth and isolation. A-L) Growth kinetics of seasonal influenza viruses in MDCK, AX-4, and hCK cells. MDCK, AX4, and hCK cells were infected with viruses at a multiplicity of infection (MOI) of 0.002. The supernatants of the infected cells were harvested at the indicated times, and virus titers were determined by means of plaque assays in hCK cells. Error bars indicate standard deviations from three independent experiments. P values were calculated by using the linear mixed model described in the Methods section (*P<0.05: **P<0.01). Red and blue asterisks indicate the comparison of hCK and AX4 cells with MDCK cells, gray asterisks indicate the comparison between the cell lines depicted in red and blue. M-P) Comparative sensitivity of hCK and AX4 cells to seasonal influenza viruses. Serial 2-fold dilutions (21 to 220) of clinical samples were prepared and inoculated into AX4 and hCK cells. Cells were observed for the development of cytopathic effect (CPE) for 7 days. Three wells were inoculated with each virus dilution. The highest dilution showing CPE in all three wells is shown by the horizontal bar. The number at the end of each horizontal bar indicates the ratio of the hCK highest dilution to the AX4 highest dilution.

FIGS. 2A-2B. Schematic overview of the generation of MDCK cells expressing markedly low levels of α2,3 -linked sialic acid and high levels of α2,6 -linked sialic acid.

FIGS. 3A-3C. Flow cytometric analysis of the cell surface expression of α2,6- and α2,3 -linked Sias. A-B) Clone 6-11 (orange line open profiles) and parental MDCK cells (black line open profiles) were incubated with biotinylated Maackia amurensis II agglutinin (MAL II) lectin (specific for α2,3 -linked sialic acid) or Sambucus nigra agglutinin (SNA) lectin (specific for α2,6 -linked Sias), followed by Alexa 488-conjugated streptavidin, and then analyzed by flow cytometry. C) Unstained cells served as negative controls (no lectin).

FIGS. 4A-4J. Characterization of MDCK cells expressing markedly low levels of α2,3 -linked sialic acid and high levels of α2,6 -linked sialic acid. MDCK cells carrying mutations in seven different β-galactoside α2,3 sialyltransferase (ST3Gal) genes were generated by using the CRISPR/Cas9 genome-editing system, as described in the Methods section The edited MDCK cells were further modified to overexpress the human β-galactoside α2,6 sialyltransferase I (ST6Gal-I) by transfection of plasmids containing the ST6Gal-I gene. The modified cell clones were selected with puromycin and blasticidin, and characterized. A-D) Flow cytometric analysis of the cell surface expression of α2,6- and α2,3 -linked Sias. Modified MDCK cells (green and red line open profiles) and parental MDCK cells (black line open profiles) were incubated with biotinylated Maackia amurensis II agglutinin (MAL II) lectin (specific for α2,3 -linked sialic acid) or Sambucus nigra agglutinin (SNA) lectin (specific for α2,6 -linked Sias), followed by Alexa 488-conjugated streptavidin, and then analyzed by flow cytometry. E-G) Immunofluorescence analysis of the expression of α2,3 -linked sialic acid. Modified MDCK and parental MDCK cells were fixed and stained with a monoclonal antibody (green) that recognizes Siaα2,3 Galβ1,4GlcNAc. Nuclei were stained with Hoechst dye (blue). H-J) Flow cytometric analysis of the cell surface expression of α2,6 -linked Sias. Modified MDCK cells (red line open profiles), parental MDCK cells (black line open profiles), and AX4 cells (blue line open profiles) were incubated with SNA lectin or Sambucus sieboldiana (SSA) lectin (specific for α2,6 -linked Sias), followed by Alexa 488-conjugated streptavidin, and then analyzed by flow cytometry. D) Flow cytometric analysis of the cell surface expression of α2,3-linked Sias. Modified MDCK cells (red line open profiles), parental MDCK cells (black line open profiles), and AX4 cells (blue line open profiles) were incubated with MAL II lectin, followed by Alexa 488-conjugated streptavidin, and then analyzed by flow cytometry.

FIGS. 5A-5F. Localization of amino acid changes in HA and NA proteins. A-C) Shown are the three-dimensional structures of A/California/04/2009 (H1N1pdm) HA (PDB ID: 3UBN), A/Wyoming/3/2003 (H3N2) HA (PDB ID: 6BKR), and B/Hong Kong/8/1973 HA (PDB ID: 2RFU) in complex with human receptor analogues. Mutations identified in this study are shown in red. Mutations in influenza A virus HA are shown with H3 numbering. D-F) Shown are the three-dimensional structures of A/California/04/2009 (H1N1pdm) NA (PDB ID: 3TI6), A/Tanzania/205/2010 (H3N2) NA (PDB ID: 4GZP), and B/Brisbane/60/2008 NA (PDB ID: 4CPM) in complex with oseltamivir carboxylate. Mutations identified in this study are shown in red. All mutations are shown with N2 numbering. Images were created with the DS Visualizer v17.2.

### DETAILED DESCRIPTION

### Definitions

A “vector” or “construct” (sometimes referred to as gene delivery or gene transfer “vehicle”) refers to a macromolecule or complex of molecules comprising a polynucleotide to be delivered to a host cell, either in vitro or in vivo. The polynucleotide to be delivered may comprise a coding sequence of interest, may comprise sequences for introducing mutations into a host cell genome, or both. Vectors include, for example, plasmids, viral vectors (such as adenoviruses, adeno-associated viruses (AAV), lentiviruses, herpesvirus and retroviruses), liposomes and other lipid-containing complexes, and other macromolecular complexes capable of mediating delivery of a polynucleotide to a host cell. Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

A “recombinant viral vector” refers to a viral vector comprising one or more heterologous genes or sequences. Since many viral vectors exhibit size constraints associated with packaging, the heterologous genes or sequences are typically introduced by replacing one or more portions of the viral genome. Such viruses may become replication-defective (biologically contained), requiring the deleted function(s) to be provided in trans during viral replication and encapsidation (by using, e.g., a helper virus or a packaging cell line carrying genes necessary for replication and/or encapsidation). Modified viral vectors in which a polynucleotide to be delivered is carried on the outside of the viral particle have also been described.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art.

By “transgene” is meant any piece of a nucleic acid molecule (for example, DNA) which is inserted by artifice into a cell either transiently or permanently, and becomes part of the organism if integrated into the genome or maintained extrachromosomally. Such a transgene may include at least a portion of an open reading frame of a gene which is partly or entirely heterologous (i.e., foreign) to the transgenic organism, or may represent at least a portion of an open reading frame of a gene homologous to an endogenous gene of the organism, which portion optionally encodes a polypeptide with substantially the same activity as the corresponding full-length polypeptide or at least one activity of the corresponding full-length polypeptide.

By “transgenic cell” is meant a cell containing a transgene. For example, a cell stably or transiently transformed with a vector containing an expression cassette is a transgenic cell that can be used to produce a population of cells having altered phenotypic characteristics. A “recombinant cell” is one which has been genetically modified, e.g., by insertion, deletion or replacement of sequences in a nonrecombinant cell by genetic engineering.

The term “wild-type” or “native” refers to a gene or gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “transduction” denotes the delivery of a polynucleotide to a recipient cell either in vivo or in vitro, via a viral vector such as a replication-defective viral vector.

The term “heterologous” as it relates to nucleic acid sequences such as gene sequences encoding a protein and control sequences, denotes sequences that are not normally joined together, and/or are not normally associated with a particular cell, e.g., are from different sources (for instance, sequences from a virus are heterologous to sequences in the genome of an uninfected cell). Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature, i.e., a heterologous promoter. Another example of a heterologous coding sequence is a construct where the coding sequence itself is not found in nature (e.g., synthetic sequences having codons different from the native gene). Similarly, a cell transformed with a construct which is not normally present in the cell would be considered heterologous.

By “DNA” is meant a polymeric form of deoxyribonucleotides (adenine, guanine, thymine, or cytosine) in double-stranded or single-stranded form found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the nontranscribed strand of DNA (i.e., the strand having the sequence complementary to the mRNA). The term captures molecules that include the four bases adenine, guanine, thymine, or cytosine, as well as molecules that include base analogues which are known in the art.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial, ” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

A “gene,” “polynucleotide,” “coding region,” “sequence,” “segment,” “fragment” or “transgene” which, in one embodiment, “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “control elements” refers collectively to promoter regions, polyadenylation signals, transcription termination sequences, upstream regulatory domains, origins of replication, internal ribosome entry sites (“IRES”), enhancers, splice junctions, and the like, which collectively provide for the replication, transcription, post-transcriptional processing and translation of a coding sequence in a recipient cell. Not all of these control elements need always be present so long as the selected coding sequence is capable of being replicated, transcribed and translated in an appropriate host cell.

The term “promoter” is used herein in its ordinary sense to refer to a nucleotide region comprising a DNA regulatory sequence, wherein the regulatory sequence is derived from a gene which is capable of binding RNA polymerase and initiating transcription of a downstream (3′ direction) coding sequence.

By “enhancer” is meant a nucleic acid sequence that, when positioned proximate to a promoter, confers increased transcription activity relative to the transcription activity resulting from the promoter in the absence of the enhancer domain.

By “operably linked” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. “Operably linked” with reference to peptide and/or polypeptide molecules is meant that two or more peptide and/or polypeptide molecules are connected in such a way as to yield a single polypeptide chain, i.e., a fusion polypeptide, having at least one property of each peptide and/or polypeptide component of the fusion. The fusion polypeptide may be chimeric, i.e., composed of heterologous molecules.

“Homology” refers to the percent of identity between two polynucleotides or two polypeptides. The correspondence between one sequence and to another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions which form stable duplexes between homologous regions, followed by digestion with single strand-specific nuclease(s), and size determination of the digested fragments. Two DNA, or two polypeptide, sequences are “substantially homologous” to each other when at least about 80%, at least about 90%, or at least about 95%of the nucleotides, or amino acids, respectively match over a defined length of the molecules, as determined using the methods above.

By “mammal” is meant any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats, rabbits and guinea pigs, and the like.

By “derived from” is meant that a nucleic acid molecule was either made or designed from a parent nucleic acid molecule, the derivative retaining substantially the same functional features of the parent nucleic acid molecule, e.g., encoding a gene product with substantially the same activity as the gene product encoded by the parent nucleic acid molecule from which it was made or designed.

By “expression construct” or “expression cassette” is meant a nucleic acid molecule that is capable of directing transcription. An expression construct includes, at the least, a promoter. Additional elements, such as an enhancer, and/or a transcription termination signal, may also be included.

The term “exogenous,” when used in relation to a protein, gene, nucleic acid, or polynucleotide in a cell or organism refers to a protein, gene, nucleic acid, or polynucleotide which has been introduced into the cell or organism by artificial or natural means. An exogenous nucleic acid may be from a different organism or cell, or it may be one or more additional copies of a nucleic acid which occurs naturally within the organism or cell. By way of a non-limiting example, an exogenous nucleic acid is in a chromosomal location different from that of natural cells, or is otherwise flanked by a different nucleic acid sequence than that found in nature.

The term “isolated” when used in relation to a nucleic acid, peptide, polypeptide, virus or cell refers to a nucleic acid sequence, peptide, polypeptide, virus or cell that is identified and separated from at least one contaminant nucleic acid, polypeptide or other biological component with which it is ordinarily associated in its natural source, e.g., so that it is not associated with in vivo substances, or is substantially purified from in vitro substances. Isolated nucleic acid, peptide, polypeptide or virus is present in a form or setting that is different from that in which it is found in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. The isolated nucleic acid molecule may be present in single-stranded or double-stranded form. When an isolated nucleic acid molecule is to be utilized to express a protein, the molecule will contain at a minimum the sense or coding strand (i.e., the molecule may single-stranded), but may contain both the sense and anti-sense strands (i.e., the molecule may be double-stranded).

As used herein, the term “recombinant nucleic acid” or “recombinant DNA sequence, molecule or segment” refers to a nucleic acid, e.g., to DNA, that has been derived or isolated from a source, that may be subsequently chemically altered in vitro, and includes, but is not limited to, a sequence that is naturally occurring, is not naturally occurring, or corresponds to naturally occurring sequences that are not positioned as they would be positioned in the native genome. An example of DNA “derived” from a source, would be a DNA sequence that is identified as a useful fragment, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use, by the methodology of genetic engineering.

The term “recombinant protein” or “recombinant polypeptide” as used herein refers to a protein molecule that is expressed from a recombinant DNA molecule.

The term “peptide”, “polypeptide” and protein” are used interchangeably herein unless otherwise distinguished.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of a selected sequence that is compared to some other sequence. Gaps (in either of the two sequences) are permitted to maximize matching; gap lengths of 15 bases or less are usually used, 6 bases or less such as 2 bases or less. When using oligonucleotides as probes or treatments, the sequence homology between the target nucleic acid and the oligonucleotide sequence is generally not less than 17 target base matches out of 20 possible oligonucleotide base pair matches (85%); not less than 9 matches out of 10 possible base pair matches (90%), or not less than 19 matches out of 20 possible base pair matches (95%).

Generally, the nucleic acid sequence homology between the polynucleotides, oligonucleotides, and fragments, and a nucleic acid sequence of interest is at least 65%, and more typically with y increasing homologies of at least about 70%, about 90%, about 95%, about 98%, and 100%.

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less such as 2 or less. Alternatively, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. The two sequences or parts thereof may be considered homologous if their amino acids are greater than or equal to 50% identical when optimally aligned using the ALIGN program.

The term “corresponds to” is used herein to mean that a polynucleotide sequence is homologous (e.g., is identical, not strictly evolutionarily related) to all or a portion of a reference polynucleotide sequence that encodes a polypeptide or its complement, or that a polypeptide sequence is identical in sequence or function to a reference polypeptide sequence. For illustration, the nucleotide sequence “TATAC” corresponds to a reference sequence “TATAC” and is complementary to a reference sequence “GTATA”.

The following terms are used to describe the sequence relationships between two or more polynucleotides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing, or may comprise a complete cDNA or gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window”, as used herein, refers to a conceptual segment of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by using local homology algorithms or by a search for similarity method, by computerized implementations of these algorithms (GAP, BESTFIT, FASTA, and TFASTA Genetics Software Package or by inspection, and the best alignment (i.e., resulting in the highest percentage of homology over the comparison window) generated by the various methods is selected.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity.

at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

As applied to polypeptides, the term “substantial identity” means that two peptide sequences, when optimally aligned, such as by the programs GAP or BESTFIT using default gap weights, share at least about 80% sequence identity, at least about 90% sequence identity, at least about 95% percent sequence identity, and or at least about 99% sequence identity.

As used herein, “substantially pure” means an object species is the predominant species present (i.e., on a molar basis it is more abundant than any other individual species in the composition), and a substantially purified fraction is a composition wherein the object species comprises at least about 50 percent (on a molar basis) of all macromolecular species present. Generally, a substantially pure composition will comprise more than about 80 percent of all macromolecular species present in the composition, more than about 85%, about 90%, about 95%, and about 99%. The object species may be purified to essential homogeneity (contaminant species cannot be detected in the composition by conventional detection methods) wherein the composition consists essentially of a single macromolecular species.

“Transfected,” “transformed” or “transgenic” is used herein to include any host cell or cell line, which has been altered or augmented by the presence of at least one recombinant DNA sequence. The host cells of the present invention are typically produced by transfection with a DNA sequence in a plasmid expression vector, as an isolated linear DNA sequence, or infection with a recombinant viral vector.

### Exemplary Cells and Modifications Thereof

Most influenza vaccines are produced in embryonated chicken eggs, but increasingly influenza vaccines are produced in other systems. MDCK (Madin-Darby Canine Kidney) cells are one of two mammalian cell lines that have been approved for influenza vaccine production. Virus production in cells may be enhanced by altering the host cell or the virus. However, for vaccine production, virus modification during passage is not advantageous.

As disclosed herein, the genome of cells, e.g., avian cells or mammalian cells including but not limited to canine, feline, equine, bovine, caprine, swine, human or non-human primate, cells may be modified to enhance influenza virus isolation, propagation, or both. For example, certain HA subtypes (HA subtypes H1-H18) may not bind well to certain species or types of cells due to the number of or composition of cell surface receptors for HA. Those cells may be modified by increasing the number of cell surface receptors or modifying the type of molecules found on cell surface receptors, or both. For example, in mammals there are about 20 sialyltransferases that transfer sialic acid residues to oligosaccharide side chains of glycoconjugates. The genes encoding one or more of those enzymes may be modified to decrease, e.g., decrease by 1%, 5%, 10%, 50%, 70%, 80%, 90% or more, or eliminate expression of the encoded enzyme, or the open reading frame for one or more of those enzymes may be expressed in the cell from an exogenously introduced expression cassette, e.g., a plasmid having that expression cassette. For example, α2,6-sialyltransferases transfer sialic acid with an α2,6 -linkage to terminal Gal (ST6GalI and II) or GalNAc (ST6GalNAcI-VI); α2,8-sialyltransferases transfer sialic acid with an α2,8-linkage (STSial-IV); and α2,3 -sialyltransferases transfer sialic acid with an α2,3-linkage to terminal Gal residues (ST3Gal). ST3GalI-II and IV transfer to the Gal residue located on terminal Galβ1-3GlcNAc, ST3GalIV and VI transfer to the Gal residue located on terminal Galβ1-4GlcNAc, ST3GalV transfers to the Gal residue located on terminal Galβ1-4Glc-Cer, and ST3 GalIII transfers to the Gal residue located on terminal Galβ1-3GlcNAc or Galβ1-3GlcNAc. Thus, each of the genes for these sialyltransferases may be employed to prepare a cell disclosed herein. In one embodiment, one or more α2,3 -sialyltransferase genes in the genome of a host cell are modified to decrease, e.g., eliminate, expression of the encoded enzyme, and one or more α2,6 -sialyltransferase genes are expressed from a recombinant expression vector introduced to the host cell. To decrease expression of a sialyltransferase, one or more vectors, or a combination of vectors and isolated protein, may be introduced to a cell. The vectors and/or protein may be part of a recombinase system that can be targeted to a specific gene in the cell, systems including CRISPR/Cas, TALEN and zinc finger nucleases.

To prepare expression cassettes (to express RNA such as gRNA or a protein including a recombinase or a sialyltransferase) for transformation herein, the recombinant DNA sequence or segment may be circular or linear, double-stranded or single-stranded. A DNA sequence which encodes an RNA sequence that is substantially complementary to a mRNA sequence encoding a gene product of interest is typically a “sense” DNA sequence cloned into a cassette in the opposite orientation (i.e., 3′ to S′ rather than 5′ to 3′). Generally, the DNA sequence or segment is in the form of chimeric DNA, such as plasmid DNA, that can also contain coding regions flanked by control sequences which promote the expression of the DNA in a cell. As used herein, “chimeric” means that a vector comprises DNA from at least two different species, or comprises DNA from the same species, which is linked or associated in a manner which does not occur in the “native” or wild-type of the species.

Aside from DNA sequences that serve as transcription units, or portions thereof, a portion of the DNA may be untranscribed, serving a regulatory or a structural function. For example, the DNA may itself comprise a promoter that is active in eukaryotic cells, e.g., mammalian cells, or in certain cell types, or may utilize a promoter already present in the genome that is the transformation target of the lymphotropic virus. Such promoters include the CMV promoter, as well as the SV40 late promoter and retroviral LTRs (long terminal repeat elements), e.g., the MMTV, RSV, MLV or HIV LTR, although many other promoter elements well known to the art may be employed.

Other elements functional in the host cells, such as introns, enhancers, polyadenylation sequences and the like, may also be a part of the recombinant DNA. Such elements may or may not be necessary for the function of the DNA, but may provide improved expression of the DNA by affecting transcription, stability of the mRNA, or the like. Such elements may be included in the DNA as desired to obtain the optimal performance of the transforming DNA in the cell.

The recombinant DNA to be introduced into the cells may contain either a selectable marker gene or a reporter gene or both to facilitate identification and selection of transformed cells from the population of cells sought to be transformed. Alternatively, the selectable marker may be carried on a separate piece of DNA and used in a co-transformation procedure. Both selectable markers and reporter genes may be flanked with appropriate regulatory sequences to enable expression in the host cells. Useful selectable markers are well known in the art and include, for example, antibiotic and herbicide-resistance genes, such as neo, hpt, dhfr. bar, aroA, puro, hyg, dapA and the like. See also, the genes listed on Table 1 of Lundquist et al. (U.S. Pat. No. 5,848,956).

Reporter genes are used for identifying potentially transformed cells and for evaluating the functionality of regulatory sequences. Reporter genes which encode for easily assayable proteins are well known in the art. In general, a reporter gene is a gene which is not present in or expressed by the recipient organism or tissue and which encodes a protein whose expression is manifested by some easily detectable property, e.g., enzymatic activity. Exemplary reporter genes include the chloramphenicol acetyl transferase gene (cat) from Tn9 of E. coli, the beta-glucuronidase gene (gus) of the uidA locus of E. coli, the green, red, or blue fluorescent protein gene, and the luciferase gene. Expression of the reporter gene is assayed at a suitable time after the DNA has been introduced into the recipient cells.

The general methods for constructing recombinant DNA which can transform target cells are well known to those skilled in the art, and the same compositions and methods of construction may be utilized to produce the DNA useful herein. For example, Sambrook et al., Molecular Cloning: A Laboratory Manual (2002) provides suitable methods of construction.

The recombinant DNA can be readily introduced into the host cells, e.g., mammalian, yeast or insect cells, by transfection with an expression vector comprising the recombinant DNA by any procedure useful for the introduction into a particular cell, e.g., physical or biological methods, to yield a transformed (transgenic) cell having the recombinant DNA so that the DNA sequence of interest is expressed by the host cell. In one embodiment, at least one of the recombinant DNA which is introduced to a cell is maintained extrachromosomally. In one embodiment, at least one recombinant DNA is stably integrated into the host cell genome.

Physical methods to introduce a recombinant DNA into a host cell include calcium-mediated methods, lipofection, particle bombardment, microinjection, electroporation, and the like. Biological methods to introduce the DNA of interest into a host cell include the use of DNA and RNA vectors. Viral vectors, e.g., retroviral or lentiviral vectors, have become a widely used method for inserting genes into eukaryotic, such as mammalian, e.g., human, cells. Other viral vectors useful to introduce genes into cells can be derived from poxviruses, e.g., vaccinia viruses, herpes viruses, adenoviruses, adeno-associated viruses, baculoviruses, and the like.

To confirm the presence of the recombinant DNA sequence in the host cell, a variety of assays may be performed. Such assays include, for example, molecular biological assays well known to those of skill in the art, such as Southern and

Northern blotting, RT-PCR and PCR; biochemical assays, such as detecting the presence or absence of a particular gene product, e.g., by immunological means (ELISAs and Western blots) or by other molecular assays.

To detect and quantitate RNA produced from introduced recombinant DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the recombinant DNA segment in question, they do not provide information as to whether the recombinant DNA segment is being expressed. Expression may be evaluated by specifically identifying the peptide products of the introduced DNA sequences or evaluating the phenotypic changes brought about by the expression of the introduced DNA segment in the host cell.

For vectors that are used to knock down or knock out expression of one or more sialyltransferases, the vectors harbor sequences that result in one or more mutations in the genome of the cell. The mutation is effective to inhibit or prevent production of at least one functional sialyltransferase. In one embodiment, the mutation is a deletion from 1, 10, 20, 50, 100, 500 and up to thousands of nucleotides, e.g., 1%, 10%, 50%, 90% or more of sequences corresponding to a sialyltransferase gene are deleted, e.g., a deletion in the coding region for a sialyltransferase, e.g., a 2,3-sialyltransferase (ST3). In one embodiment, the deleted sequences correspond to sequences with a substantial identity, e.g., at least 80% or more, e.g., 85%, 90% or 95% and up to 100% or any integer in between, nucleic acid sequence identity, to SEQ ID Nos. 5, 7, 9, 11, 13, 15 or 17, or any combination thereof. In one embodiment, the mutation is an insertion from 1, 2, 3, 5, 10, 20, 50, 100, 500 and up to thousands of nucleotides or more into sequences corresponding to a sialyltransferase gene such as an insertion into the coding region for a sialyltransferase, e.g., a 2,3-sialyltransferase (ST3). In one embodiment, the insertion is in sequences corresponding to sequences with a substantial identity, e.g., at least 80% or more, e.g., 85%, 90% or 95% and up to 100% or any integer in between, nucleic acid sequence identity, to SEQ ID Nos. 5, 7, 9, 11, 13, 15 or 17, or any combination thereof. In one embodiment, the mutation include one or more nucleotide substitutions, e.g., 1, 2, 3, 4, 5, 6, 10 or up to hundreds of nucleotide substitutions in sequences corresponding to the coding region for a sialyltransferase, e.g., a 2,3-sialyltransferase (ST3) such as substitutions in SEQ ID Nos. 5, 7, 9, 11, 13, 15 or 17, or any combination thereof. In one embodiment, a combination of insertions, nucleotide substitutions, and/or deletions in sequences with a substantial identity, e.g., at least 80% or more, e.g., 85%, 90% or 95% and up to 100% or any integer in between, nucleic acid sequence identity, SEQ ID Nos. 5, 7, 9, 11, 13, 15 or 17, or any combination thereof, are in a host cell. In one embodiment, the mutation(s) result in the host cell having reduced expression of one or more ST3 genes, e.g., encoding a protein having at least 80% or more, e.g., 85%, 90% or 95% and up to 100% or any integer in between, amino acid sequence identity to any of SEQ ID Nos. 6, 8, 10, 12, 14, 16, or 18.

In one embodiment, the host cell expresses one or more ST6 genes, e.g., encoding a protein having at least 80% or more, e.g., 85%, 90% or 95% and up to 100% or any integer in between, amino acid sequence identity to any of SEQ ID Nos. 1-4.

### The CRISPR/Cas System

The Type II CRISPR is a well characterized system that carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the

CRISPR/Cas system comprises of three steps: (i) insertion of alien DNA sequences into the CRISPR array to prevent future attacks, in a process called ‘adaptation,’ (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system. The primary products of the CRISPR loci appear to be short RNAs that contain the invader targeting sequences, and are termed guide RNAs

“Cas1” polypeptide refers to CRISPR associated (Cas) protein1. Cas1 (COG1518 in the Clusters of Orthologous Group of proteins classification system) is the best marker of the CRISPR-associated systems (CASS). Based on phylogenetic comparisons, seven distinct versions of the CRISPR-associated immune system have been identified (CASS1-7). Cas1 polypeptide used in the methods described herein can be any Cas1 polypeptide present in any prokaryote. In certain embodiments, a Cas1 polypeptide is a Cas1 polypeptide of an archaeal microorganism. In certain embodiments, a Cas1 polypeptide is a Cas1 polypeptide of a Euryarchaeota microorganism. In certain embodiments, a Cas1 polypeptide is a Cas1 polypeptide of a Crenarchaeota microorganism. In certain embodiments, a Cas1 polypeptide is a Cas1 polypeptide of a bacterium. In certain embodiments, a Cas1 polypeptide is a Cas1 polypeptide of a gram negative or gram positive bacteria. In certain embodiments, a Cas1 polypeptide is a Cas1 polypeptide of Pseudomonas aeruginosa. In certain embodiments, a Cas1 polypeptide is a Cas1 polypeptide of Aquifex acolicus. In certain embodiments, a Cas1 polypeptide is a Cas1 polypeptide that is a member of one of CASs1-7. In certain embodiments, Cas1 polypeptide is a Cas1 polypeptide that is a member of CASS3. In certain embodiments, a Cas1 polypeptide is a Cas1 polypeptide that is a member of CASS7. In certain embodiments, a Cas1 polypeptide is a Cas1 polypeptide that is a member of CASS3 or CASS7.

In some embodiments, a Cas1 polypeptide is encoded by a nucleotide sequence provided in GenBank at, e.g., GeneID number: 2781520, 1006874, 9001811, 947228, 3169280, 2650014, 1175302, 3993120, 4380485, 906625, 3165126, 905808, 1454460, 1445886, 1485099, 4274010, 888506, 3169526, 997745, 897836, or 1193018 and/or an amino acid sequence exhibiting homology (e.g., greater than 80%, 90 to 99% including 91%, 92%, 93%, 94%, 95%, 96%, 97%, 98% or 99%) to the amino acids encoded by these polynucleotides and which polypeptides function as Cas1 polypeptides.

There are three types of CRISPR/Cas systems which all incorporate RNAs and Cas proteins. Types I and III both have Cas endonucleases that process the pre-crRNAs, that, when fully processed into crRNAs, assemble a multi-Cas protein complex that is capable of cleaving nucleic acids that are complementary to the crRNA.

In type II CRISPR/Cas systems, crRNAs are produced using a different mechanism where a trans-activating RNA (tracrRNA) complementary to repeat sequences in the pre-crRNA, triggers processing by a double strand-specific RNase III in the presence of the Cas9 protein. Cas9 is then able to cleave a target DNA that is complementary to the mature crRNA however cleavage by Cas 9 is dependent both upon base-pairing between the crRNA and the target DNA, and on the presence of a short motif in the crRNA referred to as the PAM sequence (protospacer adjacent motif)). In addition, the tracrRNA must also be present as it base pairs with the crRNA at its 3′ end, and this association triggers Cas9 activity.

The Cas9 protein has at least two nuclease domains: one nuclease domain is similar to a HNH endonuclease, while the other resembles a Ruv endonuclease domain. The HNH-type domain appears to be responsible for cleaving the DNA strand that is complementary to the crRNA while the Ruv domain cleaves the non-complementary strand.

The requirement of the crRNA-tracrRNA complex can be avoided by use of an engineered “single-guide RNA” (sgRNA) that comprises the hairpin normally formed by the annealing of the crRNA and the tracrRNA (see Jinek, et al. (2012) Science 337:816 and Cong et al. (2013)

Sciencexpress/10.1126/science.1231143). In S. pyrogenes, the engineered tracrRNA:crRNA fusion, or the sgRNA, guides Cas9 to cleave the target DNA when a double strand RNA:DNA heterodimer forms between the Cas associated RNAs and the target DNA. This system comprising the Cas9 protein and an engineered sgRNA

“Cas polypeptide” encompasses a full-length Cas polypeptide, an enzymatically active fragment of a Cas polypeptide, and enzymatically active derivatives of a Cas polypeptide or fragment thereof. Suitable derivatives of a Cas polypeptide or a fragment thereof include but are not limited to mutants, fusions, covalent modifications of Cas protein or a fragment thereof.

### RNA Components of CRISPR/Cas

The Cas9 related CRISPR/Cas system comprises two RNA non-coding components: traerRNA and a pre-crRNA array containing nuclease guide sequences (spacers) interspaced by identical direct repeats (DRs). To use a CRISPR/Cas system to accomplish genome engineering, both functions of these RNAs must be present (see Cong, et al. (2013) Sciencexpress 1/10.1126/science 1231143). In some embodiments, the tracrRNA and pre-crRNAs are supplied via separate expression constructs or as separate RNAs. In other embodiments, a chimeric RNA is constructed where an engineered mature crRNA (conferring target specificity) is fused to a tracrRNA (supplying interaction with the Cas9) to create a chimeric cr-RNA-tracrRNA hybrid (also termed a single guide RNA). (see Jinek, ibid and Cong, ibid).

Chimeric or sgRNAs can be engineered to comprise a sequence complementary to any desired target. The RNAs comprise 22 bases of complementarity to a target and of the form G[n19], followed by a protospacer-adjacent motif (PAM) of the form NGG. Thus, in one method, sgRNAs can be designed by utilization of a known ZFN target in a gene of interest by (i) aligning the recognition sequence of the ZEN heterodimer with the reference sequence of the relevant genome (human, mouse, or of a particular plant species); (ii) identifying the spacer region between the ZFN half-sites; (iii) identifying the location of the motif G[N20]GG that is closest to the spacer region (when more than one such motif overlaps the spacer, the motif that is centered relative to the spacer is chosen); (iv) using that motif as the core of the sgRNA. This method advantageously relies on proven nuclease targets. Alternatively, sgRNAs can be designed to target any region of interest simply by identifying a suitable target sequence that conforms to the G[n20]GG formula.

### Donors

As noted above, insertion of an exogenous sequence (also called a “donor sequence” or “donor” or “transgene” or “gene of interest”), for example for correction of a mutant gene or for increased expression of a wild-type gene. It will be readily apparent that the donor sequence is typically not identical to the genomic sequence where it is placed. A donor sequence can contain a non-homologous sequence flanked by two regions of homology to allow for efficient HDR at the location of interest. Alternatively, a donor may have no regions of homology to the targeted location in the DNA and may be integrated by NHEJ-dependent end joining following cleavage at the target site. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded and/or double-stranded and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang, et al. (1987) Proc. Natl. Acad. Sci. USA 84:4959-4963; Nehls, et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

The donor is generally inserted so that its expression is driven by the endogenous promoter at the integration site, namely the promoter that drives expression of the endogenous gene into which the donor is inserted (e.g., highly expressed, albumin, AAVS1, HPRT, etc.). However, it will be apparent that the donor may comprise a promoter and/or enhancer, for example a constitutive promoter or an inducible or tissue specific promoter.

The donor molecule may be inserted into an endogenous gene such that all, some or none of the endogenous gene is expressed. For example, a transgene as described herein may be inserted into an albumin or other locus such that some (N-terminal and/or C-terminal to the transgene encoding the lysosomal enzyme) or none of the endogenous albumin sequences are expressed, for example as a fusion with the transgene encoding the lysosomal sequences. In other embodiments, the transgene (e.g., with or without additional coding sequences such as for albumin) is integrated into any endogenous locus, for example a safe-harbor locus. See, e.g., U.S. Patent Publication Nos. 2008/0299580; 2008/0159996; and 2010/0218264.

When endogenous sequences (endogenous or part of the transgene) are expressed with the transgene, the endogenous sequences (e.g., albumin, etc.) may be full-length sequences (wild-type or mutant) or partial sequences. In one embodiment, the endogenous sequences are functional. Non-limiting examples of the function of these full length or partial sequences (e.g., albumin) include increasing the serum half-life of the polypeptide expressed by the transgene (e.g., therapeutic gene) and/or acting as a carrier.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

Other nucleases, such as zinc finger nucleases (ZFNs), transcription activator-like effector nucleases (TALENs), or others that are specific for targeted genes and can be utilized such that the transgene construct is inserted by either homology directed repair (HDR) or by end capture during non-homologous end joining (NHEJ) driven processes.

### Exemplary Embodiments

In one embodiment, an isolated recombinant mammalian or avian cell is provided comprising a reduced amount of cell surface β-galactoside α2,3 sialyl residues and an increased amount of human β-galactoside α2,6 sialyl residues relative to a corresponding non-recombinant mammalian or avian cell. In one embodiment, the isolated recombinant cell is a non-human cell. In one embodiment, the isolated recombinant cell is a canine or primate cell. In one embodiment, the isolated recombinant cell comprises an expression cassette encoding human β-galactoside α2,6 sialyltransferase I (ST6Gal-I) or ST6Gal-II. In one embodiment, the ST6Gal-I or ST6Gal-II comprises a protein having at least 80% amino acid sequence identity to any one of SEQ ID Nos. 1-4 or 101. In one embodiment, the one or more β-galactoside α2,3 sialyltransferase genes are mutated in the recombinant cell so as to reduce the amount of cell surface β-galactoside α2,3 sialyl residues. In one embodiment, two or more of ST3Gal-1. ST3Gal-II, ST3Gal-III, ST3Gal-IV, ST3Gal-V, ST3Gal-VI, or ST3Gal-II-like genes are mutated in the recombinant cell. In one embodiment, the ST3 genes have at least 80% nucleic acid sequence identity to any one of SEQ ID Nos. 5, 7, 9, 11, 13, 15, or 17. In one embodiment, the reduction in cell surface β-galactoside α2,3 sialyl residues is the result of reduced expression of one or more ST3 sialyltransferases. In one embodiment, the one or more ST3 sialyltransferases have at least 80% amino acid sequence identity to any one of SEQ ID Nos. 6, 8, 10, 12, 14, 16, or 18. In one embodiment, influenza H3 viruses replicate more efficiently in the recombinant cell relative to the non-recombinant cell.

In one embodiment, an isolated recombinant mammalian or avian cell is provided comprising a reduced amount of cell surface β-galactoside α2,3 sialyl residues relative to a corresponding non-recombinant mammalian or avian cell. In one embodiment, one or more of ST3Gal-1, ST3Gal-II, ST3Gal-III, ST3Gal-IV, ST3Gal-V, ST3Gal-VI, or ST3Gal-II-like genes are mutated in the recombinant cell. In one embodiment, a combination of ST3Gal-I, ST3Gal-II, ST3Gal-III, ST3Gal-IV, ST3Gal-V, ST3Gal-VI, or ST3Gal-II-like genes are mutated in the recombinant cell. In one embodiment, ST3Gal-I, ST3Gal-II. ST3Gal-III, ST3Gal-IV, ST3Gal-V, ST3Gal-VI, and ST3Gal-II-like genes are mutated.

In one embodiment, a method of modifying the amount of cell surface β-galactoside α2,3 sialyl residues and human β-galactoside α2,6 sialyl residues on a mammalian or an avian cell is provided. In one embodiment, the method includes mutating one or more β-galactoside α2,3 sialyltransferase (ST3Gal) genes, and overexpressing a human β-galactoside α2,6 sialyltransferase (ST6Gal) gene, in a parental mammalian or avian cell so as to result in a modified mammalian or avian cell having a reduced amount of cell surface β-galactoside α2,3 sialyl residues and an increased amount of human-galactoside 2,6 sialyl residues on the surface of the modified cell relative to the corresponding parental cell. In one embodiment, the one or more ST3Gal genes are mutated using a genome editing system. In one embodiment, the genome editing system comprises a CRISPR/Cas9, Zinc Finger Nuclease (ZFN) or transcription activator-like effector nuclease (TALEN). In one embodiment, the mutations include one or more nucleotide insertions or one or more nucleotide deletions, or both, in one or more S73 genes. In one embodiment, the modified cell comprises an expression cassette comprising a ST6Gal open reading frame. In one embodiment, the modified cell is a kidney cell. In one embodiment, the modified cell is a canine cell. In one embodiment, the modified cell is a Madin-Darby canine kidney (MDCK) cell.

In one embodiment, a method of propagating an influenza virus is provided. The method includes infecting the recombinant cell with an influenza virus; and collecting progeny virus. In one embodiment, the influenza virus is a human influenza virus. In one embodiment, the influenza virus is an influenza A virus. In one embodiment, the influenza virus is an influenza B virus. In one embodiment, the influenza virus is a H3 virus. In one embodiment, the virus is A/H1N1, A/H3N2, a B/Yamagata-lineage influenza B virus or a B/Victoria-lineage influenza B virus.

In one embodiment, a method of isolating an influenza virus is provided which includes providing a sample from an avian or a mammal suspected of being infected with an influenza virus; and contacting the recombinant cell with the sample. In one embodiment, the method includes determining whether the sample is infected with an influenza virus. In one embodiment, the method includes identifying the HA and/or NA subtype of the virus.

In one embodiment, a method of diagnosing an influenza virus infection is provided. The method includes contacting the recombinant cell with a sample from an avian or a mammal suspected of being infected with an influenza virus; and determining if the cell is infected with virus. In one embodiment, a plaque assay is employed to determine the presence of amount of virus. In one embodiment, a nucleic acid amplification assay is employed to determine the presence of amount of virus, e.g., in the supernatant of the infected cell.

### Exemplary Sialyltransferase Sequences

Sialyltransferases in higher vertebrates are glycosyltransferases that mediate the transfer of sialic acid residues from activated sugar donors (CMP-β-Neu5Ac, CMP-β-Neu5Gc, and CMP-β-KDN) to terminal non-reducing positions of oligosaccharide chains of glycoproteins and glycolipids. The vertebrate sialyltransferase superfamily is divided into four families, ST6Gal, ST3Gal, ST6GalNAc, and ST8Sia, depending on the glycosidic linkage formed and the monosaccharide acceptor used. Members of the mammalian and avian ST6Gal family catalyze the transfer of sialic acid residues to the terminal galactose residues of the type 2 disaccharide (Gal(NAc)β1,4GlcNAc), resulting in the formation of an α2-6 glycosidic linkage. Unlike the other sialyltransferase families, this family comprises only two paralogs in the human genome named ST6GAL1 and ST6GAL2, respectively. The human ST6GAL1 gene is ubiquitously expressed in a broad variety of tissues, whereas the ST6GAL2 gene is expressed in a tissue-specific (adult brain) and stage-specific (embryonic) manner. Mammalian s16gal1 gene expression is regulated by multiple promoters governing the expression of several transcripts encoding an identical polypeptide enzyme.

In one embodiment, one or more ST3 genes in an avian or a mammlian cell, e.g., a canine or non-human primate cell, are modified so as to result in decreased expression of α-2,3-linked sialic acids on the cell surface. In one embodiment, one or more human ST6 genes are introduced to a canine or a non-human primate cell. In one embodiment, one or more ST3 genes are modified before one or more ST6 genes are introduced to the cell. In one embodiment, one or more ST6 genes are introduced before ST3 genes are modified in the cell. In one embodiment, concurrently or sequentially ST3 genes are modified and ST6 genes are introduced to the cell.

In one embodiment, the ST6Gal that is expressed comprises human ST6 (Accession No. KJ897554) comprising

(SEQ ID NO: 1)
MIHTNLKKKFSCCVLVFLLFAVICVWKEKKKGSYYDSFKLQTKE
FQVLKSLGKLAMGSDSQSYSSSSTQDPHRGRQTLGSLRGLAKAKPEASFQVWNKDSSS
KNLIPRLQKIWKNYLSMNKYKVSYKGPGPGIKFSAEALRCHLRDHVNVSMVEVTDFPF
NTSEWEGYLPKESIRTKAGPWGRCAVVSSAGSLKSSQLGREIDDHDAVLRFNGAPTAN
FQQDVGTKTTIRLMNSQLVTTEKRFLKDSLYNEGILIVWDPSVYHSDIPKWYQNPDYN
FFNNYKTYRKLHPNQPFYILKPQMPWELWDILQEISPEEIQPNPPSSGMLGIIIMMTL
CDQVDIYEFLPSKRKTDVCYYYQKFFDSACTMGAYHPLLYEKNLVKHLNQGTDEDIYL
LGKATLPGFRTIHC
or
(SEQ ID NO: 150)
MIHTNLKKKFSCCVLVFLLFAVICVWKEKKKGSYYDSFKLQTKEFQVLKSL
GKLAMGSDSQSVSSSSTQDPHRGRQTLGSLRGLAKAKPEASFQVWNKDSSS
KNLIPRLQKIWKNYLSMNKYKVSYKGPGPGIKFSAEALRCHLRDHVNVSM
VEVTDFPFNTSEWEGYLPKESIRTKAGPWGRCAVVSSAGSLKSSQLGREIDD
HDAVLRFNGAPTANFQQDVGTKTTIRLMNSQLVTTEKRFLKDSLYNEGILIV
WDPSVYHPDIPKWYQNPDYNFFNNYKTYRKLHPNQPFYILKPQMPWELWD
ILQEISPEEIQPNPPSSGMLGIIIMMTLCDQVDIYEFLPSKRRTDVCYYYQKFF
DSACTMGAYHPLLFEKNLVKHLNQGTDEDIYLLGKATLPGFRTIHC,
which is encoded by
(SEQ ID NO: 151)
ATGATTCACACCAACCTGAAGAAAAAGTTCAGCTGCTGCGTCCTGGTCT
TTCTTCTGTTTGCAGTCATCTGTGTGTGGAAGGAGAAGAAGAAAGGGAG
TTACTATGATTCCTTTAAATTGCAAACCAAGGAATTCCAGGTGTTAAAGA
GTCTGGGGAAATTGGCCATGGGGTCTGATTCCCAGTCTGTATCCTCAAGC
AGCACCCAGGACCCCCACAGGGGCCGCCAGACCCTCGGCAGTCTCAGAG
GCCTAGCCAAGGCCAAACCAGAGGCCTCCTTCCAGGTGTGGAACAAGGA
CAGCTCTTCCAAAAACCTTATCCCTAGGCTGCAAAAGATCTGGAAGAAT
TACCTAAGCATGAACAAGTACAAAGTGTCCTACAAGGGGCCAGGACCA
GGCATCAAGTTCAGTGCAGAGGCCCTGCGCTGCCACCTCCGGGACCATG
TGAATGTATCCATGGTAGAGGTCACAGATTTTCCCTTCAATACCTCTGAA
TGGGAGGGTTATCTGCCCAAGGAGAGCATTAGGACCAAGGCTGGGCCTT
GGGGCAGGTGTGCTGTTGTGTCGTCAGCGGGATCTCTGAAGTCCTCCCA
ACTAGGCAGAGAAATCGATGATCATGACGCAGTCCTGAGGTTTAATGGG
GCACCCACAGCCAACTTCCAACAAGATGTGGGCACAAAAACTACCATTC
GCCTGATGAACTCTCAGTTGGTTACCACAGAGAAGCGCTTCCTCAAAGA
CAGTTTGTACAATGAAGGAATCCTAATTGTATGGGACCCATCTGTATACC
ACCCAGATATCCCAAAGTGGTACCAGAATCCGGATTATAATTTCTTTAAC
AACTACAAGACTTATCGTAAGCTGCACCCCAATCAGCCCTTTTACATCCT
CAAGCCCCAGATGCCTTGGGAGCTATGGGACATTCTTCAAGAAATCTCC
CCAGAAGAGATTCAGCCAAACCCCCCATCCTCTGGGATGCTTGGTATCA
TCATCATGATGACGCTGTGTGACCAGGTGGATATTTATGAGTTCCTCCCA
TCCAAGCGCAGGACTGACGTGTGCTACTACTACCAGAAGTTCTTCGATA
GTGCCTGCACGATGGGTGCCTACCACCCGCTGCTCTTTGAGAAGAATTTG
GTGAAGCATCTCAACCAGGGCACAGATGAGGACATCTACCTGCTTGGAA
AAGCCACACTGCCTGGCTTCCGGACCATTCACTGCTAA;
a human ST6 (Accession No. BAC24793) comprising
(SEQ ID NO: 2)
mkphlkqwrq rmlfgifawg llfllifiyf tdsnpaepvp sslsfletrr llpvqgkqra
imgaahepsp pggldarqal prahpagsfh agpgdlqkwa qsqdgfehke ffssqvgrks
qsafypeddd yffaagqpgw hshtqgtlgf pspgepgpre gafpaaqvqr rrvkkrhrrq
rrshvleegd dgdrlyssms raflyrlwkg nvsskmlnpr lqkamkdylt ankhgvrfrg
kreaglsraq llcqlrsrar vrtldgteap fsalgwrrlv pavplsqlhp rglrscavvm
sagailnssl geeidshdav lrfnsaptrg yekdvgnktt iriinsqilt npshhfidss
lykdvilvaw dpapysanln lwykkpdynl ftpyiqhrqr npnqpfyilh pkfiwqlwdi
iqentkekiq pnppssgfig ilimmsmcre vhvyeyipsv rqtelchyhe lyydaactlg
ayhpllyekl lvqrlnmgtq gdlhrkgkvv lpgfqavhcp apspviphs;
a human ST6 (Accession No. SJL87798) comprising
(SEQ ID NO: 3)
aamgsdsqsv sssstqdphr grqtlgslrg lakakpeasf qvwnkdsssk nliprlqkiw
knylsmnkyk vsykgpgpgi kfsaealrch lrdhvnvsmv evtdfpfnts ewegylpkes
irtkagpwgr cavvssagsl kssqlgreid dhdavlrfng aptanfqqdv gtkttirlmn
sqlvttekrf lkdslynegi livwdpsvyh sdipkwyqnp dynffnnykt yrklhpnqpf
yilkpqmpwe lwdilqeisp eeiqpnppss gmlgiiimmt lcdqvdiyef lpskrktdvc
yyyqkffdsa ctmgayhpll yeknlvkhln qgtdediyll gkatlpgfrt ihc;
or
a human ST6 Gal-II
(SEQ ID NO : 4)
MKPHLKQWRQRMLFGIFAWGLLFLLIFIYFTDSNPAEPVPSSLS
FLETRRLLPVQGKQRAIMGAAHEPSPPGGLDARQALPRAHPAGSFHAGPGDLQKWAQS
QDGFEHKEFFSSQVGRKSQSAFYPEDDDYFFAAGQPGWHSHTQGTLGFPSPGEPGPRE
GAFPAAQVQRRRVKKRHRRQRRSHVLEEGDDGDRLYSSMSRAFLYRLWKGNVSSKMLN
PRLQKAMKDYLTANKHGVRFRGKREAGLSRAQLLCQLRSRARVRTLDGTEAPFSALGW
RRLVPAVPLSQLHPRGLRSCAVVMSAGAILNSSLGEEIDSHDAVLRFNSAPTRGYEKD
VGNKTTIRIINSQILTNPSHHFIDSSLYKDVILVAWDPAPYSANLNLWYKKPDYNLFT
PYIQHRQRNPNQPFYILHPKFIWQLWDIIQENTKEKIQPNPPSSGFIGILIMMSMCRE
VHVYEYIPSVRQTELCHYHELYYDAACTLGAYHPLLYEKLLVQRLNMGTQGDLHRKGK
VVLPGFQAVHCPAPSPVIPHS;
human ST6Gal1 encoded by
(SEQ ID NO: 100)
ATGATTCACACCAACCTGAAGAAAAAGTTCAGCTGCTGCGTCCTGGTCTTTCTTCTGTTTGCAGTCA
TCTGTGTGTGGAAGGAGAAGAAGAAAGGGAGTTACTATGATTCCTTTAAATTGCAAACCAAGGAA
TTCCAGGTGTTAAAGAGTCTGGGGAAATTGGCCATGGGGTCTGATTCCCAGTCTGTATCCTCAAGC
AGCACCCAGGACCCCCACAGGGGCCGCCAGACCCTCGGCAGTCTCAGAGGCCTAGCCAAGGCCAA
ACCAGAGGCCTCCTTCCAGGTGTGGAACAAGGACAGCTCTTCCAAAAACCTTATCCCTAGGCTGCA
AAAGATCTGGAAGAATTACCTAAGCATGAACAAGTACAAAGTGTCCTACAAGGGGCCAGGACCAG
GCATCAAGTTCAGTGCAGAGGCCCTGCGCTGCCACCTCCGGGACCATGTGAATGTATCCATGGTA
GAGGTCACAGATTTTCCCTTCAATACCTCTGAATGGGAGGGTTATCTGCCCAAGGAGAGCATTAGG
ACCAAGGCTGGGCCTTGGGGCAGGTGTGCTGTTGTGTCGTCAGCGGGATCTCTGAAGTCCTCCCA
ACTAGGCAGAGAAATCGATGATCATGACGCAGTCCTGAGGTTTAATGGGGCACCCACAGCCAACT
TCCAACAAGATGTGGGCACAAAAACTACCATTCGCCTGATGAACTCTCAGTTGGTTACCACAGAGA
AGCGCTTCCTCAAAGACAGTTTGTACAATGAAGGAATCCTAATTGTATGGGACCCATCTGTATACC
ACCCAGATATCCCAAAGTGGTACCAGAATCCGGATTATAATTTCTTTAACAACTACAAGACTTATCG
TAAGCTGCACCCCAATCAGCCCTTTTACATCCTCAAGCCCCAGATGCCTTGGGAGCTATGGGACAT
TCTTCAAGAAATCTCCCCAGAAGAGATTCAGCCAAACCCCCCATCCTCTGGGATGCTTGGTATCATC
ATCATGATGACGCTGTGTGACCAGGTGGATATTTATGAGTTCCTCCCATCCAAGCGCAGGACTGAC
GTGTGCTACTACTACCAGAAGTTCTTCGATAGTGCCTGCACGATGGGTGCCTACCACCCGCTGCTC
TTTGAGAAGAATTTGGTGAAGCATCTCAACCAGGGCACAGATGAGGACATCTACCTGCTTGGAAA
AGCCACACTGCCTGGCTTCCGGACCATTCACTGCTAA;
human ST6Gal1 comprising
(SEQ ID NO: 101)
MIHTNLKKKFSCCVLVFLLFAVICVWKEKKKGSYYDSFKLQTKEFQVLKSLGKLAMGSDSQSVSSSSTQD
PHRGRQTLGSLRGLAKAKPEASFQVWNKDSSSKNLIPRLQKIWKNYLSMNKYKVSYKGPGPGIKFSAE
ALRCHLRDHVNVSMVEVTDFPFNTSEWEGYLPKESIRTKAGPWGRCAVVSSAGSLKSSQLGREIDDHD
AVLRFNGAPTANFQQDVGTKTTIRLMNSQLVTTEKRFLKDSLYNEGILIVWDPSVYHPDIPKWYQNPD
YNFFNNYKTYRKLHPNQPFYILKPQMPWELWDILQEISPEEIQPNPPSSGMLGIIIMMTLCDQVDIYEFL
PSKRRTDVCYYYQKFFDSACTMGAYHPLLFEKNLVKHLNQGTDEDIYLLGKATLPGFRTIHC;

or a protein having at least 80%, 85%, 87%, 90%, 92%, 95%, 96%, 97%, 98%, or 99% amino acid sequence identity to any one of SEQ ID Nos. 1-4, 101 or 150 or a nucleotide sequence having at least 80%, 85%, 87%, 90%, 92%, 95%, 96%, 97%, 98%, or 99% nucleotide acid sequence identity to any one of SEQ ID Nos. 100 or 151.

In one embodiment, the ST3 gene that is mutated has at least 80%, 85%, 87%, 90%, 92%, 95%, 96%, 97%, 98%, or 99% nucleic acid sequence identity to a canine ST3GalI comprising

(Accession No. XM_022426722)
(SEQ ID NO: 5)
gtcaggtctc agaaaagtct agaataaggc ttacaggcac tttgttcagt tgtggaacac
atggaaactt catacaccgc ccccctcctt gcagaccgga gagctctctg ctctaatttg
ggcacaggcg cccaaccttg ggccccagag gaagcctgtt ctactcccag ggccaacgct
caattgcttg gtaacttggc acccacactg ttccaggctc tggtctcagt ttcactctct
gcaaaatgag aggctcagag ttccgctgac atggcctcgg ggccgttaaa acctttcttc
atgaagacag cctgcaccct tcctgcttct gctccaggtc catcctcaga accttccaga
aagtcctggc acgtcagata acagggccaa cccggcagtg atgccccacc ccccacccct
tcccttactc atagagcctg ctccagactc tcagagccca cacccacttg gtgaagtcat
ttgccagtaa ttcttcgcac tggacattga gaggtttcag accccagaag tctcaggcgc
tgggtctgaa agtgggcaga gcccaggtga catttgtgga gactctcagt ggtgcgtata
gccgccggga ccattttcag actcaacctt tctgacctgg aaatgccaat agaagtaatc
atcatcgcca ggggctgtgg tgagcaaatg cttggagctc tgtggccagg gcagtttcat
ttgaggacca gagatggaca atccctcagc ctactgagat gaagaaactg agtctcagag
aggttaagga actcccccga ggttgcacca ctgaggaaga ttgacctgac ttcccaagac
catacatctg gtaaaccgga acctgcacct gcgccatctc aagcctactc tggaggcccg
aggctaattg gcagagtttg agggctgaga tgacagacaa ccctcagtgc cttcatcggg
cgggctgctt acctgcacat ccctggtgac agcatgggaa agaccgcttc taattaagcg
tcatcacaca tcaccctttt ccggaggaag agaggcaaag acagctcccc tctatcctgc
actgtgaacc ttcctccgag gtcctcccct cacccccgag agtccttgcc ctgtcaccaa
gattaattac ccctcaaccc ctttggatgg caaaggcagt cattttatta agtttgatta
aagcttcaag agacattgcc ggatgtttca ggactgctga caaagcagcc tgcttgtttc
ctgaaaagac caattatatg caagaagcgt cagccccact ccccgagggg tccacttagc
ctccgaccac cacagggtga tgtccagcgc accggtgtgg ccatcacctg gcgggagtga
ggtcggccgt agcaggtctg gaggcccccc tgcaagtccc tgcctcaccc tgtgactgag
cctctcgtct ctgggaattt tgttccaaat tccccgtcta ccaggtgtga tttcctccag
ccccaccagc cctgggaggc gcccatccag agagcagaga tggtgaccat gagaaagagg
actctcaaag tgctcaccct cctcgtcctc ttcatcttcc tcacctcctt cttcctgaat
tactcccaca ccatggtgac caccacctgg tttcccaagc agatggttgt cgagctctca
gagaacttta agaagttcat gaaatacact cacaggcctt gcacctgtgc ccgctgcatc
gggcagcaga gggtctcggc ctggttcgat gagaggttca accggtccat gcagccgctg
ctgaccgccc agaacgccct cttggaggag gacacctaca gctggtggct gaggctccag
cgggagaagc aacccaacaa cttgaacgat accatcaggg agctgttcca ggtggtgccc
gggaacgtgg accccctgct ggagaagagg tcggtgggct gccggcgctg cgcagtcgtg
ggcaactctg gcaacctccg agagtcctgg tacgggcctc agatcgacag ccacgacttc
gtgctcagga tgaacaaggc ccccacggcg ggcttcgaga tggatgtcgg gagcaagacc
acccaccacc tggtgtaccc cgagagcttc agggagctgg cggagaatgt cagcatggtc
ctggtgccct tcaagaccac cgacctggag tgggtggtca gtgccaccac cacaggcacc
atctctcaca cctatgttcc tgttcctgca aagatcaaag tgaaaaagga taagatcctc
atctaccacc cggccttcat caagtacgtc ttcgacagct ggctgcaggg ccacgggcgg
tacccgtcca ccggcatcct ctccgtcatc ttctcgctgc acatctgcga cgaggtggac
ttgtacggct tcggggcaga cagtaagggg aactggcatc actactggga gaacaatcca
tcggcggggg ctttccgcaa gaccggggtg cacgacggag actttgagtc caacgtgacg
gccaccttgg cgtccatcaa taagatccgg atatttaagg ggagatgacg ctgccgagga
gcaccggagc ccgcctcttt ggccagcccc agcctctgct ggagccgatc tgtgctgggg
gctttgaggg ccagcctcgg gggcgtgttc aggtgcccct cgtgccccct cgcaccccga
catttggcag catcgactca gcaaggcccc agagccccgc tgggtctgca gagcgagtgt
cagaactggt cttggatggg gacacccccc ctccacctcc ctgcaccgcc actgctgtcc
tggagcaggg ggcaggtggg gagacgcggg aggaggtccg gtctccaaac gctcaagtca
tttcggcttt ggggggcaga gggggagata tcgaagactc tgtgttctaa agtgaaggac
actggccctg ggcagagggg tcccgggctg actctctgac tctgatgctt gctgggtgag
gacgacctcg ggacagagcc acatcgggcc acgtggacgc tgggggtgag aaggcacctt
cctctgcacg atgcccggcc cctccctcac cgcctctgcg gtcttcccga agctcctccg
tggcggccag gagaggcgcc ctgcgccgag ggtcctcaca gatgcttggc caagtgtttc
agactccagc aggtgtcttc ttcgcaagct gaggctccct tgagtgatcg atctttgtgg
ccataaatga tggctgagag cagatctgac tacttccacg tgcctttgtg tctgggggag
agctgtgcgc attggctgaa ataaggcaaa agccttaatt cgggagtggg gagctccccc
ctctccccgc ccccagcaat gccaccccct tgctctggag ctgggtaaca tctttactag
tttcctgagg cggtgccgga gctggaatga agctaggatg atgctcaacg gcgtccagag
gtgcatcagc cgcggcacgt cattcttagg ttcacagact gatggatttg gggcccgtga
ccttccaagg ccacacacag aacctccacc agccagcact ttgccccagg agcccaccct
ccaggccaag tccctccggg ccacctgcac agtgtcctgg ggtttgtggg gccatcccag
gaacatgacc ctcggagcgg gagaagacat acctcatcct ctcacttctg ggctgctctg
attcctcctc atgattattt attgattttt ttttaattca gctgacattg cgtgcacttt
gtggctccgt ggctggtcct gatgttttaa ttaagcttgc tctcgcttca cctggcagcc
ggggcatggg gggcttaagt caaggtttgc aggagtcctc agacttggga gggggatgca
tatctagggc ttgggggctc gtcggtgggc aaatgccagg gtttcaggtt ggtaggttct
ccgagtgctc tgtacctcct tcccctcagc tctgcctccc ttctccatca ttgcgggttg
ggatcattct caggctaaga atctgcaaaa cccagtgaga cggccttggg gccaggtggg
gagtggagtc acacaggcag ggcagcgatg gttggctccg gttgctgaca ctgaatcaga
aaatccacgt ttcctattga gagcatttcc taacaggcca ctgctacttc gaggaggtgt
gacagtgtcc tggctgtcac gggggcccgt ctcctgtgct ccttgcagca gcactccggc
aagttctgct ctcctggctt cagtctctgc ctctgcaaac ggagagggag gcatttgggg
agctcagggg gccacaggtg gtttcagggg gttcacaagc gtccccagag agagcacacc
acgcccgtgt tttaccccaa gcctgtgtgt atgagcatat attcctgggg agcggtgccg
tagcgttcat tcaattgtca gaggttcaga acccagacaa tggtttacaa aaacacgaaa
cgacagcaag caaacaaaaa cccgttcgag ctttaaaatc ctccaacact tagactctct
tgacccctaa aggctcataa atcactacga ggtcagcaag ctgatcataa atcccaaaga
ctatatagca ggcatctgtg ggttggctga tgaatccgct gagagaagtg ggtgcggagc
ggcctccgag ctcctccccg agtcctggaa gagccgagtc cagtcagagc tgatgggtcc
agagcgctca gcctgcgtcg gggctgtgct gagtcaggac aggtccccag agtaaggggg
ggggtctctc tccctcccca ccccactgag ccgtccaggc tgttgagagg acttcccacc
tgccttccca tgtaacactt gagaaaagcg aggcccggag aagggaagtg gcttgcaaag
tgaacccgtg gtgctggaca acgcaagtca gaccctttga gcaaagatag agcacgattg
ctacatgttg aacacacctc gaggtgggct ttcccagccg gcttccatcc agcctcccca
aggactgtga ccgggcacag gtgggctcct caggctgcac gtggtggccc ccgcaagctc
cggtgtctgc cttttccccc catccctcca gtgagggaga cacccttgct cttccctcag
ccccagcaaa cttgtctttg tgttcgttgg cttagcaaag gggtctgacc cgaatgcacc
aacgggcttc agccccctgg catctgtacc tggggtaagg gtggctgaga atccaaccag
agtatttccc ccagagctcg tggctgtggg caggttaacg agaaccaccc gcccccccca
ctcggaggag gctcctgaat gcccacccca gtgttgggct cccagcctag ggccctgctt
gctcctcaag cacagagctc ccttggaggc cagtcctgga cccctgggca gggagcagag
gcatccctgc ctctctcagt gctgagcccg aagccaggaa gaccagggcc gaggagggag
agagagattt taacaccctc cccggccctg gggagccagg agctggagtc acctgccgca
aatgcaaggc ctggttccac gtgcccccct ccgcccctgt cctcaccccc ttgttagggt
gggtttgtag cgtctggctc ccagagccat caggcaaaaa agaaagttct taaagagatg
cttttagggg cctgtgctta gtgggagaag ccagattcaa gaaaatggga cttaaaacgg
ggaagccaga cgagaaggtg gggggggggt tgtccgggtg tgattcttac aagtgctgag
gccagaagga tggaccctct ccaggtccag agtagagtgg agtcaacggg gaatatttag
gactgtgatg tttgcagtaa tagtcctctt caaggcagcc tggacacaaa gcaaaatcgg
aaacagaaaa gaactcacca tcccttgcgt gggtaggacc aaaacagctt tctccaggga
ggcgcaagcc ctgagtggcc gccgtctggc caaagccggc ctcgtgccat cgggaaccgg
ccaccgccca tgcccctccg gagagctcgg ggtcgtcatc gtgtccacca cgggcatctg
gtcggaagca aacgttccca caggccactg gctggtcatc gtcccctgtg gtctgggctg
accggtttga gaccaagtcc gctgccttgc tccggcctgt cctgaagaca ccagcgcccc
ggcctgtggg gtgccccgtg atgcctccat gcagggaaga gcaggggacc agggaggaag
agcagagaca ggtgaagcga cagtccccgc gtcccagcct cagcattcgc atcctcttgg
cccctacttt tcctctccgc ccagcagaca tctgccctgc ccttgccctt gaccccattg
ctgcgcttcc ctcaaggacg ggcctggcct tggtggccac ctgcggacag ccctgcgccc
gacgcccgct tcaccccggg gcccgggtct ggaggggccg cccccaggac gaacgcggct
gccccacggg gccggcccct caccggcttc gcgtccaagc caaagtttct cgagcacttt
tttgttcttt gcaatcatgt tgggttcatt gttggtgttt taaaattttg cttccctctc
cctctggcct cgctcctgtg tgtgttttgt agccgagcgc taacctggat gcttttttga
atgacctttg caagagcctg ccttcctcgg cctctgctct gttttattta ttgttgaata
tttccaatga tccaaatcaa agtgaattaa aacaaagcta ttttatcgtt;

a ST3 gene encoding a protein having at least 80%, 85%, 87%, 90%, 92%, 95%. 96%, 97%, 98%, or 99% amino acid sequence identity to

(SEQ ID NO: 6; Accession No. XP_022282430)
mvtmrkrtlk vltllvlfif ltsfflnysh tmvtttwfpk qmvvelsenf kkfmkythrp
ctcarcigqq rvsawfderf nrsmqpllta qnalleedty swwlrlqrek qpnnlndtir
elfqvvpgnv dpllekrsvg crrcavvgns gnlreswygp qidshdfvlr mnkaptagfe
mdvgsktthh lvypesfrel aenvsmvlvp fkttdlewvv satttgtish tyvpvpakik
vkkdkiliyh pafikyvfds wlqghgryps tgilsvifsl hicdevdlyg fgadskgnwh
hywennpsag afrktgvhdg dfesnvtatl asinkirifk gr;

a ST3 gene having at least 80%, 85%, 87%, 90%, 92%, 95%, 96%, 97%, 98%, or 99% nucleic acid sequence identity to a canine ST3GalII gene comprising

(Accession No. XM_014114023)
(SEQ ID NO: 7)
cggctcgctc cgggagggca gagccggcaa gggcgggact ggcctgctgc gggcggacgg
gggagccgcg gagctaacgg gtccggacgt cgccagcggc ggagctttct gcacgggcgg
aggcggcggc aggagcggca agcatggccc gcgcggcgcc cggcgctgac tgagggtcat
gctgcggcag cctggcgcct gacgagtgag ggggaacctg ccaggggatt actgctagca
cgagctaaca gcaggacagc aggcctcagg ggagtgggga tgggcatagc agcctgcctg
caacgctggg ccagtggcac caaggaggcc caggccctgg cagggagctc ggcacacgct
cagctgaccc agacagcatt caaagcaggt ccctgctgag cagcagctgt tcccaggccc
tggaggaccg caaacgcctc agcaaccaca tttggggctg tgaccatcag gagaagggac
caacacctcg aatgggaagc agaagggcag aacctgctgc caggaggatc ttggtctctg
cagcaaagtc ttccgacacc cgtggagctt cttgggtttc tcctctcagg actggggcct
tgtttcctcc tctggtggag ggggtaggcc aggagggcca gctgaggtgg ggacgactct
cggatggaat ccaggccact tgaagcccat gggctctctg ctgtgattgg ggttcgaggg
cttccccact gcaggggacg aggggccgcc tccgtgtctg ctcatgaacc acaaggaccc
cgaatgctcc agactggacc atttcgagcc accaaagagt ggggccccca gagctggcag
ccagcagctc caagggacta gagagctggg atggactgac ctccccctca ccagggcatt
aggagagtca gagcctctgt ggcccagcta gtgacagaga gacccgatga ggccataagc
tggcacccga gtgactcagg gacaggagag ccactcctgc ctactgtgct ttctctttac
attgagggag ggtgtggtaa gggacccctg cctctgttcc ctcctcagca cgtgccccta
tgccctttgc acgtggtgcc aggagacgca ggctacactg tggctggccc atcagtgggc
tgggaaggga gtggccacgg tgacacccac ccgcccacca ggctggtggt ctagcccccc
ggggcgccaa accatgaagt gctccctgcg ggtgtggttc ctctctgtgg ccttcctgct
ggtgttcatc atgtcactgc tctttaccta ctcccaccac agcatggcca ccttgcccta
cctggactca ggggccctgg gcggtaccca ccgggtgaag ctggtgcctg gctatgctgg
tctgcagcgc ctcagcaagg aggggctcac cggtaagagc tgtgcctgcc gccgctgcat
gggtgacact ggcgcctctg actggtttga cagccacttc aacagcaaca tttcccctgt
gtggacccga gagaatatgg atctgcctcc agatgtccag aggtggtgga tgatgctgca
gccccagttc aagtcacaca acaccaacga ggtactggag aagctgttcc agatagtacc
aggcgagaac ccctaccgtt tccgggaccc ccaccagtgc cggcgctgtg cggtagtggg
gaactcaggc aacctgcggg gctctggcta cggcccagat gtggatgggc ataacttcat
catgaggatg aatcaggcgc caaccgtggg ctttgagcag gatgttggca gccgaactac
ccaccatttc atgtaccccg agagtgccaa gaaccttcct gccaacgtca gctttgtgtt
ggtgcccttc aaagctctgg acctactatg gattgccagc gctttgtcca cagggcaaat
cagattcacc tatgcgccag tgaagtcctt ccttcgagtg gacaaagaaa aggttcagat
ctacaacccg gcattcttca agtacatcca cgaccggtgg acagagcatc acgggcggta
cccttccaca gggatgctgg tgctcttctt tgccctgcat gtttgtgatg aggtgaacgt
gtacgggttc ggggccgaca gccggggcaa ctggcaccac tactgggaga ataaccggta
tgcgggcgag ttccagaaga cgggagtgca cgacgccgac ttcgaggccc acatcatcga
catgctggcc aaggccagca agatcgaggt ctaccgaggc aactgagccc ggcctcgccg
cgacccttcc ggcccagccg cgaggctgcg acgctcgctc cgcagccggg actcccggcc
agcccgaggg cggcggcctt ggcgagggcg cggcgcccgg ttgggcgtct ccagccctgg
gagcgacgcc aagtcgcggt ctggaccaat catgctgcaa gtccagcgag cgccggctgt
cccccgccaa tcaggagact ttgggggctg gcccaggcct ggcacccaat cagcgctgca
gtgggagcgg aggctctttc tcccagccaa tcatgcgact caaggagaac ttccggcgct
gggcccggtc tcctccaatc aatggccttc ggaggcgggc cggccgccgc tgaatcccca
ctcccctatg ctttgggtag gattttattt tatgcttttt aaggagtagt gattggttcc
ggcctcagtg gagtactttc ctcaggctct gcgggaggag tgttggtggc ctgtcggcgg
tactcggcca ggggcaccga ggaggaagcg ggggggaagg tgcggggcag cagcggctgg
gcctcccttg gccgggggcc cctcgcgacc tcggggcggg cgggggggcg ggggcgttgg
cctcccgctt ctggaggtcc gggggaaatc aggtggtttc cggggagcgc gctttcatcc
ccgggaagag ctagatccct ctcaaacctt ttcaggcctc agagcactct agaccgcgta
tttcctttat ctgtcgggcc cagatgggtg agtgtaaacc caccaaagaa aggcagtgga
gcgtgggtcc cctcgtcctc ccttgtcccc atccccacct ttggccaccc tatgggatgg
ccttcctgac caggacattg agcatcccac ctggaaacta gaactgtatt cacccgttgc
tagggctcgg agttcgccga tggcctaact cgagcgcagg ggtaaggaac agggcagggg
acccggaagc cccgtcactt cagatgtaag gtgcttctca ctctcgtgta ctctcgcggc
cccttactgt tcgcccacaa cttttttagt gtcccttctt aagccctggg cctcctcacc
agcctgctcg tcctgggatt gggggtgggg ggtggggcac tgctggcttc tccaaccccc
tacccctcct cgctcgtctt tagccggctc tagggagagg aaggcaggct ggagatgggg
agcccagctg cctggtgcat gcaccgtttt cctccgccca tcaccccaaa gaggagtagg
aaacctcttg cttgggggtg gaatttgctt tggtctccta atttagttaa cttgaggtta
ccagggatgg ctgaccaaca aagattcttt taaaattcca ggctggccat gcaaattgct
gggatcctag ctggggagga gtcgactgac ttgcccgcct tgcatgtctc ctctcctgcc
cctgcgtccc ctccctctgc cacgctcact tcctgcctca tctctccaac ccattttcca
ttttcagctc tagaagggca gggacgctta caaacaggag ttacatctgg aagttacttc
caagactgaa cccagcttaa gtccctagag gaagctgctg atgatattct cacccttcaa
ggttggggaa gtttcggaag gggaaagtgc ttctgtgaag cttccaaacc actaatagga
tcccccttcc caacaatgag gaacacaaac accacccttt atcttagttg ataccaccaa
gcagcctcct ggccattggg gtaattcctg cagctggctg gggtaaccag caggggagta
tattagaaga ggattggggc agggcagtgg gcacccctaa agttaatata ttgagaactt
agcttaaacc taagtcttag ttccttccca attccaaaag taggaggagc aacgagtggg
ggtggatttg ggggggccta tcctggaatg cctctctcag ggcttccccc accattttag
agagtcaagg caccagccat tcatgccagt ctcctctcag tgcttcctga agaggctgtt
tggagtgttc ggaaaatgaa aaaaacaatg caattatgcc aaacagtatt gagcagaata
atttatttct tttttgttcc tttttcttct ttttgttttg tttaaaacat taataaatcc
cctttctgga agaggtaggt cccagcatcc agcccagatc tccttttctg caatagttat
ttaaacaaat gtttgtttgt ttttttattt tcttcccttt ctctctcttt ctgaattaaa
aaaaaagaaa actccta;

a ST3 gene encoding a protein having at least 80%, 85%, 87%, 90%, 92%, 95%, 96%, 97%, 98%, or 99% amino acid sequence identity to

(Accession No. XP_013969498)
(SEQ ID NO: 8)
mkcslrvwfl svafllvfim sllftyshhs matlpyldsg alggthrvkl vpgyaglgrl
skegltgksc acrrcmgdtg asdwfdshfn snispvwtre nmdlppdvqr wwmmlqpqfk
shntnevlek lfqivpgenp yrfrdphqcr rcavvgnsgn lrgsgygpdv dghnfimrmn
qaptvgfeqd vgsrtthhfm ypesaknlpa nvsfvlvpfk aldllwiasa lstgqirfty
apvksflrvd kekvqiynpa ffkyihdrwt ehhgrypstg mlvlffalhv cdevnvygfg
adsrgnwhhy wennryagef rktgvhdadf eahiidmlak askievyrgn;

a ST3 gene having at least 80%, 85%, 87%, 90%, 92%, 95%, 96%, 97%, 98%, or 99% nucleic acid sequence identity to a canine ST3GalIII gene comprising

(Accession No. XM_025420404)
(SEQ ID NO: 9)
ttgtggtcg cgctccgccc gccgctgcgt ccccaccatg gcggcgcccg tgcagcccac
cgcgtcgtgg gcgcccgccg ggctcccccg cggctgtggc ggccgcccgc gcctcggcct
ccgcctcccc gcccgcgccg gcccgggcgc cgcctccccg ctgcctccgt ctccgctgcg
gtcatgtaqg aaatcgtaaa tcatgtgaag atgggactct tggtatttgt acgcaatctg
ctgctagccc tctgcctttt tctggtactg ggatttttgt attattctgc gtggaagctg
catttactcc agtgggagga ctccaattca gtggttcttt cctttgactc cgctggacaa
acactaggct cagagtatga tcggttgggt ttcctcctga agctggactc taaactgcct
gctgagttag ccaccaagta tgcaaacttt tcagagggag cttgcaagcc tggctatgct
tcggccttga tgactgccat ctttccccgg ttctccaagc cagcacccat gttcctggat
gactctttcc gcaagtgggc taagattcgg gagtttgtgc cgccttttgg gatcaaaggt
caagacaatc tgatcaaagc catcttgtca gtcaccaaag agtaccgcct gacccctgcc
ttggacagcc tcagctgccg ccgctgcatc atcgtgggca acggaggtgt cctagccaac
aagtctctqg ggtcacgaat tgatgactat gacattgtqg tcagactgaa ctccgcacca
gtgaaaggct ttgagaagga cgtgggcagc aaaactacac tgcgcatcac ctaccctgag
ggcgccatgc agcggcctga gcaatatgaa cgcgattctc tatttgtcct cgctggcttc
aagtggcagg acttcaagtq gttgaagtac atcgtctaca aggagagagt gctctgggcc
cgcagggata cctgccaatc tgtctgggcc catccccctc tcccctccac cagctgtcac
cagccacccc aggggaggag tcctgcagag ttcaggccat tcttcttcca atacccgagc
ctcctactgg aggagaatga tgacagacag cctctggcga caagtgcatc agatggcttc
tggaaatccg tggccacacg agtgcccaag gagccccctg agattcgcat cctcaacccg
tacttcatcc aggaagccgc cttcaccctc atcggactgc ccttcaacaa cggcctcatg
ggccgcggga acatcccgac ccttggcagt gtggcagtga ccatggcgct acacggctgt
gatgaggtgg cagtcgcagg ctttggctac gacatgagca cacccaacgc gcccctgcac
tactatgaga ccgtgcgcat ggcagccatc aaagaggtca ccagcgactc agctcaaggc
tgccaaatcc agtggacaca tggaagcctc atctttcctg acctcccaga aatgcttttt
ctgttgacca ctccttcctc tttgaaactt ttcctgctca gactgtcctg gacacacaat
atccagcgag agaaagagtt tctgcgcaag ctggtgaagg cgcgcgtcat caccgaccta
accagcggca tctgaggtgg gcccagcaca tggccacgga ggtcctggca ccgccaagag
gaagccgcag ccactgccac ctgtccactt cattggcctc ggtctggctc tgcctgaaag
gcgcaggagt cttcagaccc agagaaggac agtgccaagg gg;

a ST3 gene encoding a protein having at least 80%, 85%, 87%, 90%, 92%, 95%, 96%, 97%, 98%, or 99% amino acid sequence identity to

(Accession No. XP_025276189)
(SEQ ID NO: 10)
mgllvfvrnl llalclflvl gflyysawkl hllqwedsns vvlsfdsagq tlgseydrlg
fllkldsklp aelatkyanf segackpgya salmtaifpr fskpapmfld dsfrkwarir
efvppfgikg qdnlikails vtkeyrltpa ldslscrrci ivgnggvlan kslgsriddy
divvrlnsap vkgfekdvgs kttlritype gamqrpeqye rdslfvlagf kwqdfkwlky
ivykervlwa rrdtcqsvwa hpplpstsch qppqgrgpae frpfffqyps llleenddrq
platsasdgf wksvatrvpk eppeirilnp yfigeaaftl iglpfnnglm grgniptlgs
vavtmalhgc devavagfgy dmstpnaplh yyetvrmaai kevtsdsaqg cqiqwthgsl
ifpdlpemlf llttpsslkl fllrlswthn iqrekeflrk lvkarvitdl tsgi;

a ST3 gene which has at least 80%, 85%, 87%, 90%, 92%, 95%, 96%, 97%, 98%, 40 or 99% nucleic acid sequence identity to a canine ST3GalIV gene comprising

(Accession No. XM_014113293)
(SEQ ID NO: 11)
gccctacagg cccgagctgc cggggtcggg cctccccggg ttcccgctcc caggtcctcc
tggacacacc ggcctggcct ggctcccggg gaactctcgt ctgctagcgg ggagcctccc
tccgcctcgc ccacgggcac ccctcccacc cagtatcctt ggcctcttgc aggtggcccg
aggcagccgg gatgacagct ctccccagga accctgctac cctctgagaa acatgatcag
caagtcccgc tggaagctcc tggccatgtt ggctctggtc ctggtcgtca tggtgtggta
ttccatctcc cgagaagaca ggtacattga acttttttat tttcccatcc cagagaagaa
ggaaccgtgc ttccagggtg aggcagagag aaaggcctct aagctctttg gcaactactc
ccgagatcag cccatcttcc tgcagatgaa ggattatttc tgggtcaaga caccgtctgc
ctacgagctg ccctatggga ccaaggggag cgaagacctg ctcctccggg ttctagccat
caccagctac tccattccag agagcatcca gagtctcaag tgtcgccgct gcgtggtggt
gggcaatggg catcggctgc gcaacagctc gctgggagat gccatcaaca agtacgacgt
ggtcatcaga ctgaacaacg cccccgtggc tggctacgag ggtgacgtgg gctcgaagac
caccatgcgt ctcttctacc cggagtcagc ccacttcaac cccaaagtgg agaacaaccc
agacacactt ctcgtcctgg tggccttcaa ggcaatggac ttccactgaa ttgagaccat
cctgagtgat aagaagaggg tacgaaaggg cttctggaag cagcctcccc tcatctggga
cgtcaacccc aggcaggttc ggattctcaa ccctttcttt atggagattg cagctgacaa
actgctgaac ctgccaatga aacagccacg caagatttcc cagaagccca ccacgggcct
gctggccatc acgctggctc tccacctctg cgacctggtg cacatcgccg gcttcggcta
cccggacgcc cacaacagga agcagaccat tcactactat gaacagatca cgctcaagtc
catggcgggg tcaggccaca acgtctccca ggaggccctg gccatcaagc ggatgctgga
gatcggagca gtcaagaacc tcacgttctt ctgacgggga caggagctct agccgtcagt
ctgcccgccc tgccgcctaa gcgaccaacc acggctgtgg gggcgccggc gtgacctgct
tggattcccc ctccccgtgt ggagaggggg cctggtacag gcgggccctg agatggggcc
gcgcccctgg ctgctcttgg ggcggccgga tccagtcagg gtggaggccc cgggtggcgg
gaggccttcc gaggcgcggg gtgtgtggct gaggcacccc ttctcaccag ccccgggagc
ttatttaatg ggctatttaa ttaaaagggt aggaatgtgc ctcgggctgg tcccatggca
tccggaaacg ggggcatagc acagtggtct gcccactgtg gataaaaaca cacaagtgct
tggcccacta gagcctagag ccagagcagg cctcccagga gggcaggggc gtctggagcg
ggtgggtgcc ctccagagag gggctgctac ctcccagcgg gcatgggaag agcattggga
tgaagtccca cggagaatag gacctcatgt agaaaagagg tttgaaacct aacattaaac
tattttttcc taaaacggaa;

a ST3 gene encoding a protein having at least 80%, 85%, 87%, 90%, 92%, 95%, 96%, 97%, 98%, or 99% amino acid sequence identity to

(Accession No. XP_013968768)
(SEQ ID NO: 12)
misksrwkll amlalvlvvm vwysisredr yielfyfpip ekkepcfqge aerkasklfg
nysrdqpifl qmkdyfwvkt psayelpygt kgsedlllrv laitsysipe siqslkcrrc
vvvgnghrlr nsslgdaink ydvvirlnna pvagyegdvg skttmrlfvp esahfnpkve
nnpdtllvlv afkamdfhwi etilsdkkrv rkgfwkqppl iwdvnprqvr ilnpffmeia
adkllnlpmk qprkisqkpt tgllaitlal hlcdlvhiag fgypdahnrk qtihyyeqit
lksmagsghn vsqealaikr mleigavknl tff;

a ST3 gene having at least 80%, 85%, 87%, 90%, 92%, 95%, 96%, 97%, 98%, or 99% nucleic acid sequence identity to a canine GalV comprising

(Accession No. XM_022404744)
(SEQ ID NO: 13)
cgctctggaa ccacttacag ccacctggtg catcctcctt tggggtgcgt ttggagggcc
tggttcctgc tcagccacat cttctgccac tttcaccagc aatgcccagt gagtataact
atgtaaaact gagaagcgat cgctcaagac cctctctgca atggtacacc cgagctcaaa
acaagatgag aagacccaac ttgttgttaa aagacatcct taagtgtaca ttgcttgtgt
ttggagtgtg gatcctttat attctcaagt taaattatac tactgaagaa tgtgacatga
aaaaaatgca ttatgtggac ccagaccgtg taaagagagc tcagaaatat gctcagcaag
tcttgcaaaa ggagtgccga cccaagtttg cgaagaagtc gatggcgcag ttgttcgagc
acaggtacag cacggactta ccacctttcg tgaaggagac ccccaaaatg aatgaaaccg
agtacaagta tgatcctcct tttggattcc gaaagttctc cagtgaagtc cagaccctgt
tggaaatact gcccgagcat gacatgcccg aacacttgag agcaaagagc tgtaggcgtt
gtgtggtcat cggaagcggt ggcatactcc acggactagc actgggccag gccctcaacc
agttcgatgt ggtgataagg ttaaacagtg caccagttga gggatattct gagcatgttg
gtaataaaac tactataagg atgacttatc cagagggcgc gccactgtct gaccttgaat
attattccaa tgacttgttt gttgctgttt tattcaagag tgttgacttc aactggcttc
aagcaatggt aaaaaatgaa accctgccat tttgggtgcg gctcttcttt tggaagcaga
tggcgaaaaa aatcccacta cagccaaaac atttcaggat tttgaatcca gttattatca
aagagactgc ctttgacatc cttcaatact cagagcccca gtcaaggttc tggggccgag
ataagaacgt gcccaccatt ggtgtcattg ccgttgtctt agccacacat ctgtgtgatg
aagtcagctt ggcaggcttt ggatatgacc tcaatcaacc caaaacacct ttgcactact
ttgacaatct ctgcatggct gccataaact ttcaaaccat gcataatgtg acaacggaga
ccaggttcct cctcaagctg gtcaaagagg gcgtggtgaa ggatctcagc ggaggcatcc
attgtgaatt ttgaacacag ggaaacctca tgtgacaatg caactctgac tctgaaggct
gtttttcgta gccttctcga tgcagcgcat cctgcaaaat acttagaggt gcagctgggg
ttttt;

a ST3 gene encoding a protein having at least 80%, 85%, 87%, 90%, 92%, 95%, 96%, 97%, 98%, or 99% amino acid sequence identity to

(Accession No. XP_022260452)
(SEQ ID NO: 14)
mpseynyvkl rsdrsrpslq wytraqnkmr rpnlllkdil kctllvfgvw ilyilklnyt
teecdmkkmh yvdpdrvkra qkyaqqvlqk ecrpkfakks maqlfehrys tdlppfvket
pkmneaeyky dppfgfrkfs sevqtlleil pehdmpehlr akscrrcvvi gsggilhgla
lggalnqfdv virlnsapve gysehvgnkt tirmtypega plsdleyysn dlfvavlfks
vdfnwlqamv knetlpfwvr lffwkqvaek iplqpkhfri lnpviiketa fdilqysepq
srfwgrdknv ptigviavvl athlcdevsl agfgydlnqp ktplhyfdnl cmaamnfqtm
hnvttetrfl lklvkegvvk dlsggihcef;

a ST3 gene having at least 80%, 85%, 87%, 90%, 92%, 95%, 96%, 97%, 98%, or 99% nucleic acid sequence identity to a canine GalVI comprising

(Accession No. XM_005639375)
(SEQ ID NO: 15)
ggtcgattgc cccttggctg ctgtggaggc tgtgatgacc tccagggccg cggccctccg
ggcgatgctt ctccaggggc tgaggccaac gcagaactcc cgtggcaccc actcggactc
gcggcgtgtt cacatgtggg gttttattaa atcctcccac caaccgtgtg agacaggaac
agttagcccc ggtgtgtccg ccaagattgc cccgcacaag tggctccgga tggatcacac
gaagcacttg caagtgaaga agcagcacag ccctttatct tgggctattt cctgtggaga
gactccaaca atttagcagc caggctcctg ggcctctggg accctcacca catcacatcc
ttcaccttca ggagcagagc gcctttggga aacagacttc taaaagtgca ggtgggccag
ccatgagagg gtacctagtg gccatattcc tgagtgctgt ctttctctat tatgtgctgc
attgtatatt gtggggaaca aacatctatt gggtgccacc tgtgaaaatg aagcggagaa
ataagatcca gccttgttta gcgaagccag cttttgcctc tctcctgagg tttcatcagt
ttcacccttt tctgtgtgca gctgatttta aaaagattgc ttccttgtat ggtagcgata
agtttgatct gccctatggg ataagaacat cagcggaata ttttcgactc gctctttcaa
aactacagag ttgtgatctc tttgatgagt ttgacaatgt gccgtgtaaa aagtgcatgg
tggttggtaa tggaggagtt ctgaagaata agacattagg agaaaaaatt gactcctatg
atgtcataat aagaatgaat aatggtcctg ttttaggaca tgaagaggaa gttgggagaa
ggacaacctt ccgacttttt tatccagaat ctgttttttc agatcccaat cacaatgatc
ctaatactac agcgattctc actgctttta agccgcttga cttaaagtag ctgtgggaag
tgttgacggg tggcaaaata aacactaatg gtttttggaa gaaaccagct ttaaacttga
tctacaaacc ttatcaaatc agaatattag atcctttcat tatcagaatg gcagcttatg
aactgcttca cttcccaaaa gtgtttccca aaaaccagaa acccaaacac ccaacaacag
gaattattgc catcacgctg gcctttcaca tatgtcacga agttcacctt gctggtttta
aatacaattt ttctgacctc aagagccctt tacactatta tgggaacgca accatgtctt
tgatgaataa gaatgcgtat cacaatgtga cagcggaaca gctctttttg aaggacattc
tagaaaaaaa ctttgtaatc aacttgactg aagattgacc ctacagactc tgcagatgat
gctaagagta ttagttttat ttttatactg caatttttag tttattttta aatatattgg
atgcacttgt caaaaaattg tgtatagtca gtctgttgct gcctggtgat tcataaccac
cagcttaatt tctgtgaata tatttaattt ataaaaacca agaagatatg cttagatatc
cgggaagttt tgattgcgtt ggttttaaaa caaccttagt tctctgaagt gtttttaaac
atctttttta atagttactt catctttgac ttctgagggc atgtaacgtc caagtaaggg
gctttagctt gaccaccaca aactctgaac agagttggtg gcggattcgg ctactgtaaa
ttggtgggga atagccatgt gattgtgcaa actggaaccg gtttaggcaa gtatcgagtt
cctttttact gaacccgagg aaacggattt gaatcttaaa gcaggcccaa ccatagcagt
aggtacggtt atgaaatcta agatcataat ggtttcatta agcttttttt cctgtaagta
aaccagatta taaaatgaaa ggtgtttgtt tttaaggtag aggaaacagg ctacatgtga
aattctggat gagtaaacaa cctaggaatg caattactaa agtctggtgg ctgcattatt
ttaaagttca tacaaagaag cagagctagg ccacctcaag gagacagttc ttaaacgtca
tcttttgcct gccttaatat gttaaaattt ggaagtttac tatttgaaat aggaaagatg
aatacggcac agtaggtaaa tccttcagac tcctcaggct gtttttggat ttaaatagtc
ctttcgtgaa aaatctcact tgtccacggt gaaatcccat cttcaaaggg aaggcttacc
cggctaccta gggtgcatca gagaagagtc ctgctggatg cagacaagtc aaaaccagcc
tgtccaacaa acgtgcgccc gtctctcttc tcaaagaggg atggaatgaa cagctctcag
aagaggtaag agttgaagga cttgttatcc tctgagcgat aatcgtcatg gagagacact
gctggtgttc ctgaaaacca gcctgcctct gagtctcaga gacaaaatat gagagcagcc
actgggataa atcgtgaagc acggcataag gggggagaag cctcgtagtt gattgaaccc
atgtctacgt ggcttcagct gattcccctg taacggaggt ggaaagttcc cgcacgtaca
cagctgcacg ctgcagcctg gcggctggga ttccatgggt ggactcattc agggtacaaa
gacagtcctg gctgcaaagt gaaaaacccc aggtggcatt ttcaagtgtt tatggactga
aataatggct gtacggtatc tggcggatgc tcaacttgag gaatcggcat ttttgtacag
tgggagctga ggctataaac ctcagcgtgg cttcacataa gccagaagaa actctcagcc
cgatacatat gtacaattta ttaaaaacac atgaacacat taaaatctca ctatttatac
aatctacatt ctagcaacat atacaaatac cgagtgacta cagtacatgc cgaggtaaga
aaagtacatt cggggagact atcactgaca ctcaagccat ttttatttcc aatatgtttt
gctttcacct ttcccagtgc caaaaaaaaa aaaaaaaaa;

a ST3 gene4 encoding a protein having at least 80%, 85%, 87%, 90%, 92%, 95%, 96%, 97%, 98%, or 99% amino acid sequence identity to

(Accession No. XP_005639432)
(SEQ ID NO: 16)
mrgylvaifl savflyyvln cilwgtniyw vppvemkrrn kiqpclakpa fasllrfhqf
hpflcaadfk kiaslygsdk fdlpygirts aeyfrlalsk lqscdlfdef dnvpckkcvv
vgnggvlknk tlgekidsvd viirmnngpv lgheeevgrr ttfrlfypes vfsdpnhndp
nttailtafk pldlkwlwev ltggkintng fwkkpalnli ykpyqirild pfiirmaaye
llhfpkvfpk nqkpknpttg iiaitlafhi chevhlagfk vnfsdlkspl hyygnatmsl
mnknayhnvt aeqlflkdil eknfvinlte d;

a ST3 gene having at least 80%, 85%, 87%, 90%, 92%, 95%, 96%, 97%, 98%, or 99% nucleic acid sequence identity to a canine ST3GalII-like comprising

(Accession No. XM_025469036)
(SEQ ID NO: 17)
aaagacttca ctgggtatca gtctcctttg ggagaccaca ggacacgtgt cacctctccc
atcctctcag cctccagccc agaccttggc agagttcctt ttaggagtta gcaagtggct
gaggaggcaa gaggtgccag agccaatcta ctatctgctg ggggatgatt gccagggcca
gagatgaggg ctcaatactt gaagtggggt ctggtagctg cctgtatagt tacgttatgg
ctgatgatga tgaacttcct ggaccaggag ttcaaacaga atgacttccc taaaaagaca
agaatacaat tatgccactg ccccaggaac tctttcagaa agtgtaggtg ttcgtttgag
atccgcaagt gctctgcctg cctccgcgta cgtggaacgt ctgtctggtt tgatgaacgc
ttcgaaacgg ctattgagcc tgtgcagaga ccagaagatc ccatatcctc tgatgctcta
atattgtggt tgggtatcca atcaaagagg gagtttgaga ctcagaagcc aatagaagag
cctcctgggc aaccactggg ctacgtggag tccagttgtc ggacctgtgc agtggttgga
aactcaaggt gcctacgggg ctctggccat ggattcagga ttaaccaaaa tgacatggtc
ctcaggatga accaggcccc cgtccaagga tttgagatgg atgtggggaa cacaaccacc
atgcgcataa tgtaccccga tatggctagc acgcagaatc ctggcaccaa attgctgctg
cttcctctga attcatctgg tctaaagtgg tttatggaag tactacagga acagagcttc
agaaagccca taaaccctgg atttcagata gtccagtttc ctgatggaag taacacgagc
aaagacgagg tcttggtgat cagcctcacc tttcttcagt acatccagga tcattggctg
cgaaaacgtc atcgttttcc atccttgggg tttgtgggtc tgttatatgc cctgcacact
tgtgaccagg tatccttatt tggttttggg acagatcagc tcatgaggtg gtcccattac
tgggatgata aatatcggtt cgagagtaac atgcacagtt tcaaagaaga gcagaagctc
atcctccagc tgcaatgtaa ggggaagatt gttatctaca gctgacatgt ttctgtcctg
ttcagcccac tggaggcccc aggaggctga caggtagtca aggggaccac agagtgtcag
agagggactg gggcttcaag tggaccctgg atatagatca gtctgctgct aaataaaact
acagcttatt tctccca;

or
a ST3 gene that encodes a protein having at least 80%, 85%, 87%, 90%, 92%, 95%, 96%, 97%, 98%, or 99% amino acid sequence identity to

(Accession No. XP_025324821)
(SEQ ID NO: 18)
mragylkwql vaacivtlwl mmmnfldqef kqndfpkktr iqlchcprns frkcrcsfei
rkcsaclrvr gtsvwfderf etaiepvqrp edpissdali lwlgvqskre fetqkpieep
pgqplgyves scrtcavvgn srclrgsghg frinqndmvl rmnqapvqgf emdvgntttm
rimypdmast qnpgtkllll plnssglkwf mevlqeqsfr kpinpgfqiv qfpggsntsk
devlvisltf lqyiqdhwlr krhrfpslgf vgllyalhtc dqvslfgfgt dqlmrwshyw
ddkyrfesnm hsfkeeqkli lqlqcegkiv iys.

The invention will be further described by the following non-limiting example.

### EXAMPLE

### Methods

Cells. MDCK and AX4 cells were maintained in Eagle's minimal essential media (MEM) containing 5% newborn calf serum (NCS) or 10% fetal calf serum (FCS). All cells were incubated at 37° C. with 5% CO₂, and regularly tested for mycoplasma contamination by using PCR and were confirmed to be mycoplasma-free.

Clinical specimens. Respiratory specimens were obtained from patients with influenza-like symptoms who visited clinics in Yokohama city, Japan during the 2017-2018 influenza season, and were submitted to the Yokohama City Institute of Public Health for virus isolation. These clinical specimens were collected under the National Epidemiological Surveillance of Infectious Diseases program in Japan. Respiratory specimens were also obtained from patients with influenza-like symptoms who visited clinics in Tokyo, Japan during the 2013-2014, 2015-2016, 2016-2017, and 2017-2018 seasons, and were submitted to the Division of Virology, Department of Microbiology and Immunology, Institute of Medical Science, the University of Tokyo for virus isolation. These specimens were collected by attending physicians after informed consent was obtained. Our research protocol was approved by the Research Ethics Review Committee of the Institute of Medical Science of the University of Tokyo (approval no. 26-42-0822). Samples that were positive by real-time RT-PCR (see below) or rapid diagnostic kits were used in this study.

Viruses. Human influenza viruses were propagated in hCK cells in MEM containing 1 ug of L-1-Tosylamide-2-phenylethyl chloromethyl ketone (TPCK)-trypsin/ml.

Real-time RT-PCR. RNA was extracted from clinical specimens by using the Simply RNA Tissue Kit (Promega) or RNeasy Mini Kit (Qiagen). Amplification and detection by real-time PCR were performed with the Applied Biosystems 7900HT Fast Real-Time PCR System (Applied Biosystems) or StepOnePlus Real-Time PCR System (Applied Biosystems). RT-PCR was carried out using the QuantiTect multiplex RT-PCR kit (Qiagen) or QuantiTect Probe RT-PCR Kit (Qiagen). The probes contained oligonucleotides with the 6-carboxyfluorescein (FAM) or the hexacholoro-6-carboxyfluorescein (HEX) reporter dye at the 5′ end, and the Black Hole Quencher-1 (BHQ-1) or 6-carboxytetramethylrhodamine (TAMRA) quencher dye at the 3′ end. The list of primers and probes used is provided in Table 5.

Virus isolation. MDCK, AX4, and hCK cells grown in 12-well plates were inoculated with 0.2 mL per well of the clinical samples and incubated at 34° C. for at least 30 minutes. One microliter of MEM containing 2.5 μg/mL acetylated trypsin was then added to cells. The cultures were then incubated for up to 7 days, until CPE was evident. Cell culture supernatants were harvested and subjected to hemagglutination assays using guinea pig red blood cells (see below).

Hemagglutination assay. Viruses (50 μL) were serially diluted with 50 μL of PBS in a microtiter plate. An equal volume (i.e., 50 μL) of a 0.75% (vol/vol) guinea pig red blood cell suspension was added to each well. The plates were kept at 4° C. and hemagglutination was assessed after a 90-minute incubation.

RT-PCR and sequencing of viral genes. Viral RNA was extracted from 140 ul of culture supernatants using the QIAamp Viral RNA Mini kit (Qiagen). Samples were amplified using the SuperScript III One-step RT-PCR System with Platinum Taq High Fidelity DNA Polymerase (Invitrogen) and specific primers of HA or NA genes. PCR products were then analyzed by means of 1.5% agarose gel electrophoresis in tris-buffer, and target bands were visualized by staining with GelRed (Biotium). The PCR products were purified and subjected to direct sequencing. The level of mutation frequencies were examined based on the height of the waves at each position on the sequencing chromatogram. The detection limit for a minor population was 10%-20%. The list of primers used is provided in Table 5.

Serial passages of human influenza viruses. Ten-fold serial dilutions (10¹ to 10⁶)of viruses were prepared in MEM. Each dilution was inoculated into MDCK, AX4, and hCK cell monolayers in 24-well culture plates using one well per dilution. The plates were incubated at 33° C. for 3 days. The end point was taken as the highest dilution of the sample showing CPE. Culture supernatants were harvested from wells inoculated with the 10-fold higher concentration of dilution than the end point dilution, and were used for the next round of infection. Viruses sampled after the first and sixth passages in the supernatants of each cell were subjected to sequence analysis.

Statistical analysis. Data are expressed as the mean±SD. For the analysis of the growth curve data, we performed a linear mixed effects analysis. As fixed effects, the different cell lines, and the time of the measurement (with an interaction term between those fixed effects), were used. As random effects, intercepts for the individual animals were used. The virus titer values were transformed to the log 10 scale, and the R statistical package (www.r-project.org), lme4 (Bates et al., 2015), and the lsmeans package (Lenth, 2016) for the group comparisons, were used. The p-values were adjusted using Holm's method and considered significant if less than 0.05.

### Generation of MDCK Cells Expressing Markedly Low Levels of α2,3 -Linked Sialic Acid and High Levels of α2,6 -Linked Sialic Acid

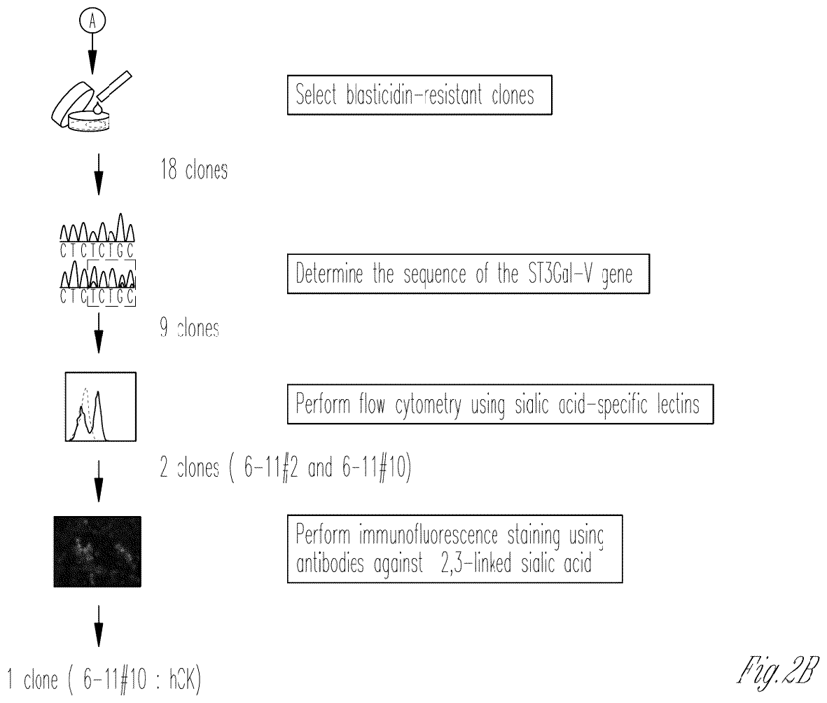

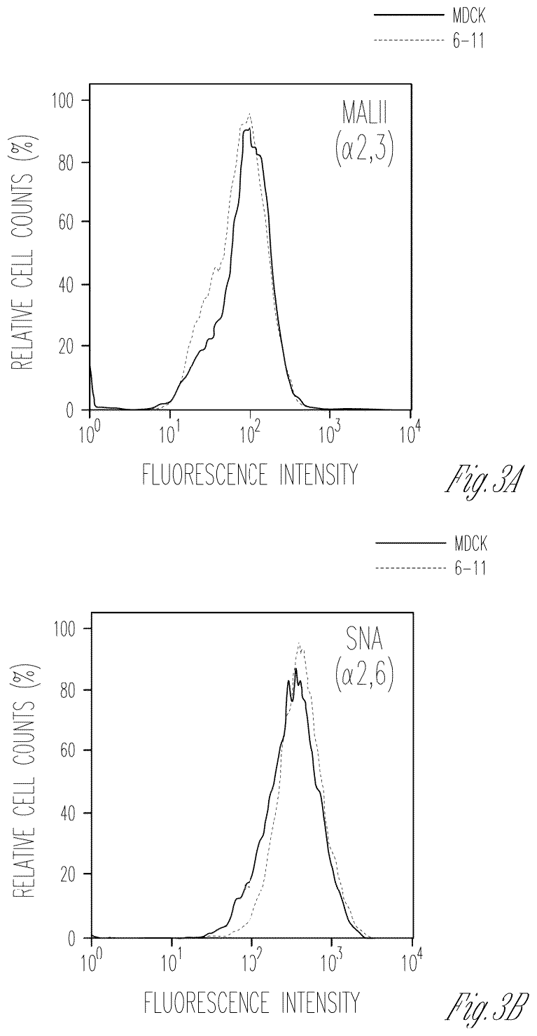

To mimic the expression pattern of sialic acid (Sia) molecules on the surface of human upper airway epithelial cells, we first attempted to knockout the β-galactoside α2,3 sialyltransferase (ST3Gal) genes, whose products catalyze the transfer of Sia with an α2,3 -linkage to terminal galactose (Gal) residues, by using the clustered regularly interspaced short palindromic repeats (CRISPR)/CRISPR-associated protein 9 (Cas9) gene editing system (Cong et al., 2013; Jinek et al., 2012; Han et al., 2018; Shalem et al., 2014). Dogs have seven different ST3Gal proteins (ST3Gal-I,-II,-III,-IV,-V,-VI, and ST3Gal-II-like protein) each of which is encoded by a distinct gene. ST3Gal-I,-II,-III,-IV, and-VI use oligosaccharides on glycoproteins, or on glycoproteins and glycolipids, as acceptor substrates, whereas ST3Gal-V utilizes oligosaccharides on glycolipids only (Takashima and Tsuji, 2011). A previous study reported that N-linked glycoprotein is required for productive entry of influenza viruses into host cells (Chu and Whittaker, 2004). Therefore, to inhibit the transfer of α2,3 -linked Sias to glycoproteins, MDCK cells were transfected with a mixture of six plasmids, each containing a Cas9 gene expression cassette and an expression cassette for the individual guide RNA (gRNA) targeting the ST3Gal-I,-II,-III,-IV,-VI, or ST3Gal-II-like protein gene (FIG. 2) After transfection, puromycin was added to the cells, and 33 drug-resistant clones were randomly picked up. Genomic DNA analysis revealed that only one clone (6-11) contained mutations in the gRNA target regions for the six ST3Gal genes (data not shown). Cell surface Sias was measured by flow cytometry using the Maackia ammurensis II agglutinin (MAL II) lectin specific for α2,3 -linked Sias and the Sambucus nigra agglutinin (SNA) lectin specific for α2,6 -linked Sias Unexpectedly, the reactivity with MALII was very similar between the parental MDCK cells and clone 6-11, indicating that the clone still expressed high levels of α2,3 -linked Sias (FIG. 3). This may have been due to the compensatory activity of ST3Gal-V.

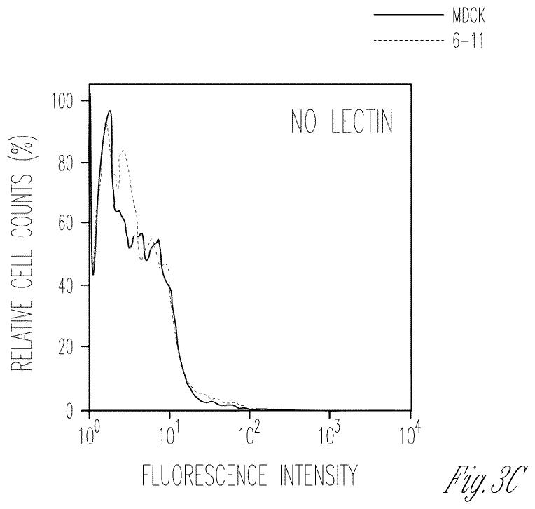

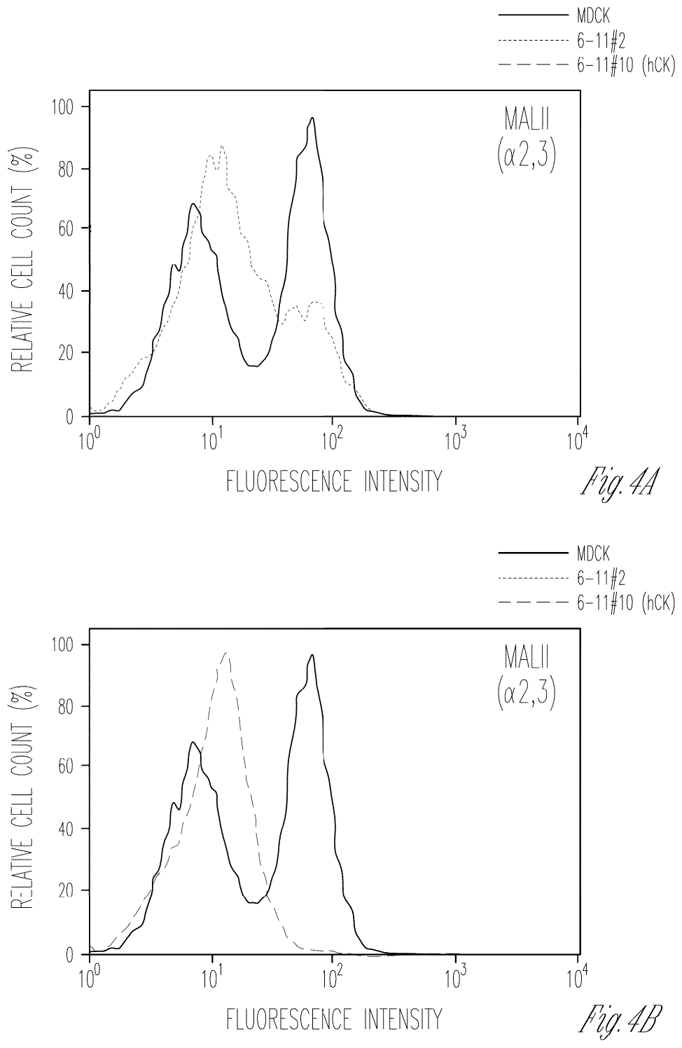

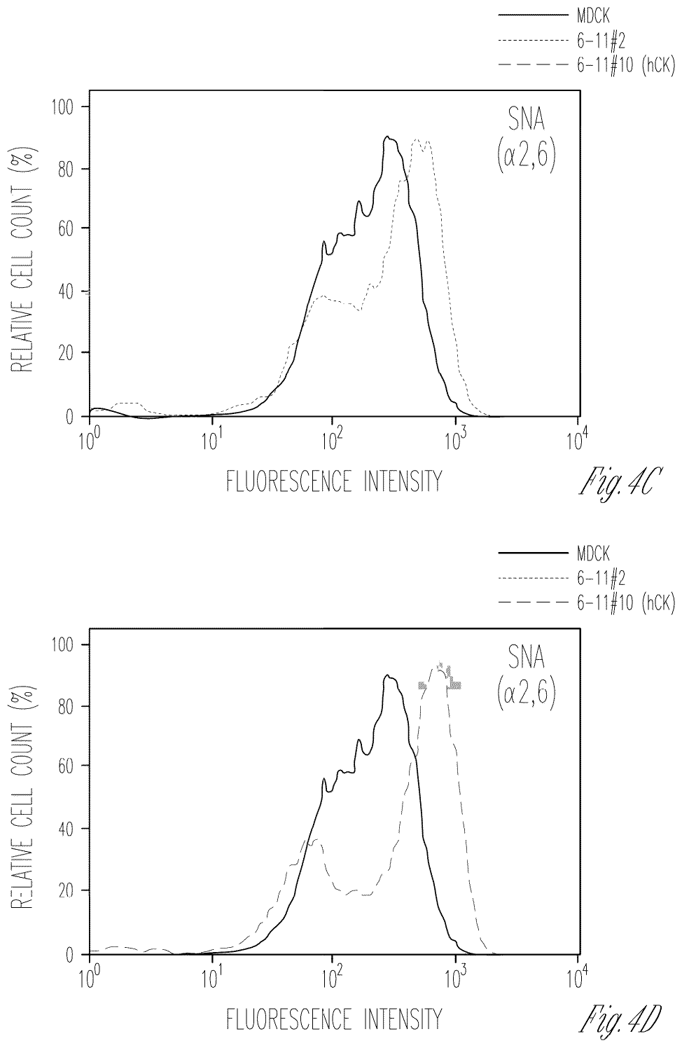

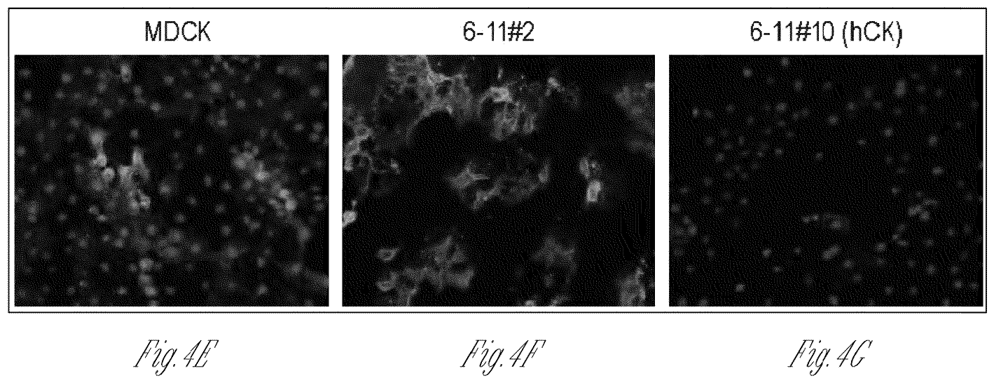

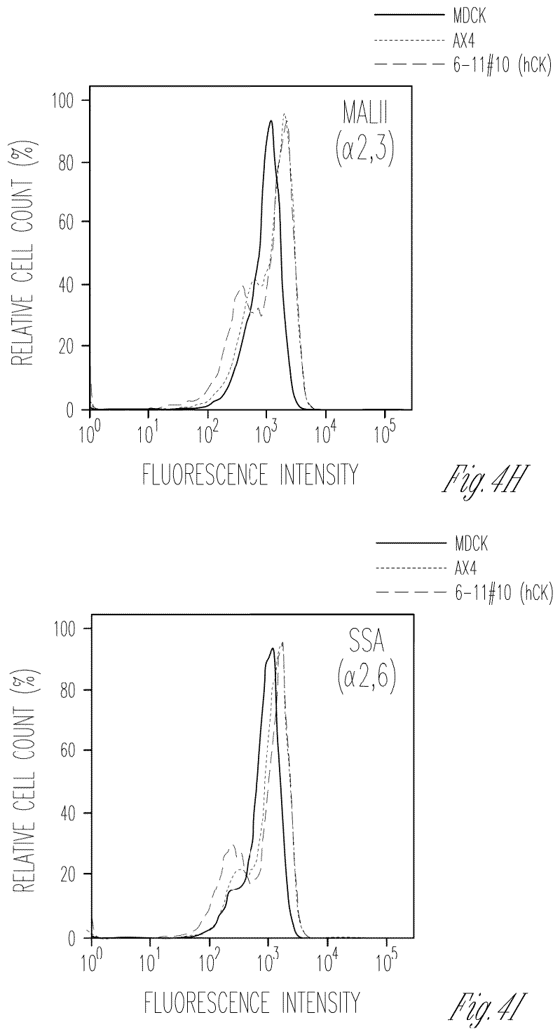

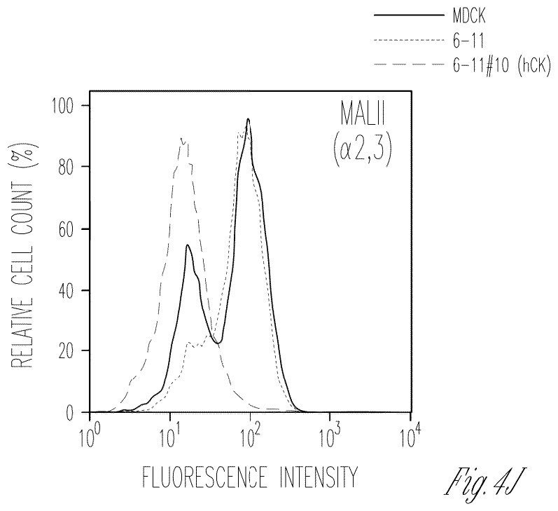

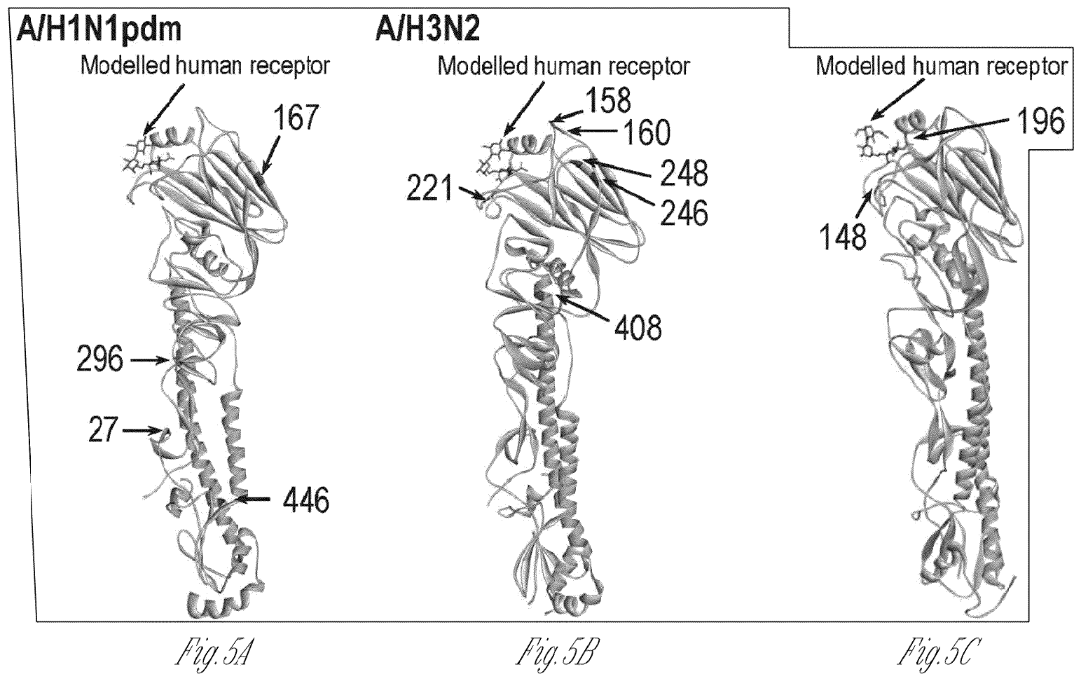

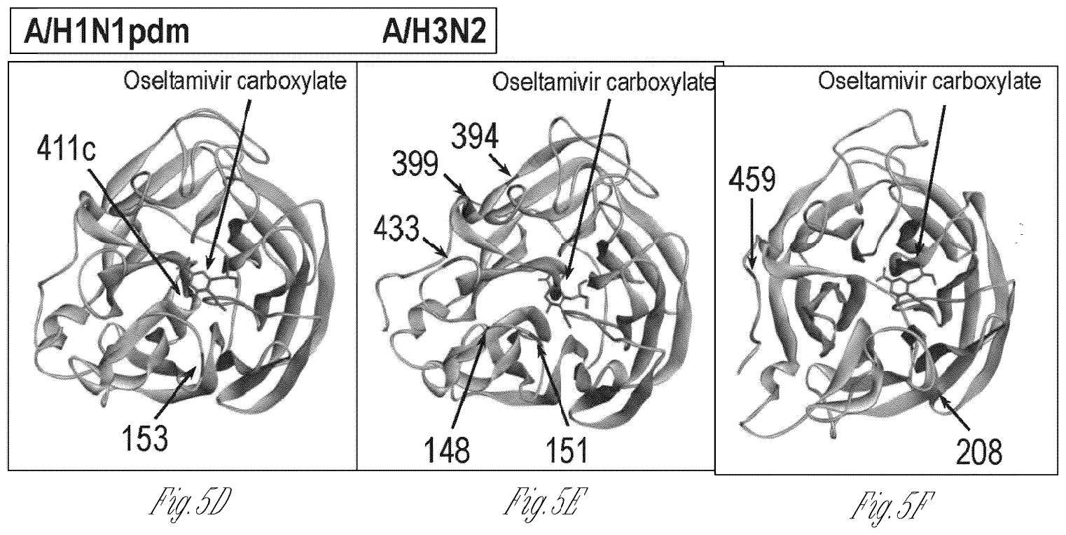

To inhibit the transfer of α2,3 -linked Sias more efficiently and to express high levels of α2,6 -linked Sias on the cell surface, clone 6-11 was co-transfected with a plasmid encoding human β-galactoside α2,6 sialyltransferase I (ST6Gal-I), which catalyzes the addition of α2,6 -linked Sia to Gal-containing glycans, and a plasmid containing expression cassettes for Cas9 and a gRNA targeting ST3Gal-V. Eighteen cell clones were selected with blasticidin and subjected to genomic DNA analysis. Among the drug-resistant clones, 9 possessed a mutation in the gRNA target region for the ST3Gal-V gene (data not shown). Flow cytometric analysis using the MAL II and SNA lectins revealed that two (clones 6-11 #2 and 6-11 #10) of the nine clones had markedly decreased expression of α2,3 -linked Sias compared with the parental MDCK cells and higher expression levels of α2,6 -linked Sias than those of the parental cells (Sy FIG. 4a; data for only clones 6-11 #2 and 6-11 #10 are shown). Terminal Sia is attached to several types of oligosaccharide structures on glycoproteins or glycolipids, such as Galβ1,4GlcNAc (GlcNAc; N-acetylglucosamine), Galβ1,3GalNAc (GalNAc; N-acetylgalactosamine), and Galβ1,4Glc (Glc; glucose) (Takashima and Tsuji, 2011). The MAL II lectin preferentially recognizes the Siaα2,3 Galβ1,3GalNAc structure (Hidari et al., 2013). To assess whether the two clones express different types of α2,3 -linked oligosaccharide structures on the cell surface, an indirect immunofluorescence assay (IFA) analysis was performed using a monoclonal antibody against Siaα2,3 Galβ1,4GlcNAc7. IFA showed that levels of Siaα2,3Galβ1,4GlcNAc were undetectable or markedly low in one (6-11 #10) of these two clones (FIG. 4b), suggesting that in clone 6-11 #10, multiple types of oligosaccharides containing terminal α2,3 -linked Sias are expressed at lower levels than in the parental cells. Next, the cell surface expression levels of α2,6 -linked Sias on AX4 cells and clone 6-11 #10 were compared by using the SNA and Sambucus sieboldiana (SSA) lectins, both of which recognize the Siaα2,6Gal or Siaα2,6 GalNAc structure (Shibuya et al., 1989). Flow cytometric analysis indicated that there were no differences in the expression level of α2,6 -linked Sias between AX4 cells and clone 6-11 #10 (FIG. 4c). However, the expression level of α2,3 -linked Sias, as measured by using the MAL II lectin, was markedly lower in clone 6-11 #10 compared to AX4 cells (FIG. 4d). It was confirmed that clone 6-11 #10 contained the desired mutations in the gRNA target regions for the seven ST3Gal genes (Table 1). These results show that clone 6-11 #10 expresses mainly human virus receptors and limited amounts of avian virus receptors. The resulting clone, 6-11 #10, was designated hCK, and subsequently expanded for further analysis.

TABLE 1
Amino acid changes in the HA and NA of viruses analyzed
after passages in MDCK, AX4, or hCK cells^{a}.

| | | | HA^{b} | | | NA^{c} | | |
|---|---|---|---|---|---|---|---|---|
| Virus type | Sample ID | Cell | P1 | P6 | P10 | P1 | P6 | P10 |
| H1N1pdm | BB139 | MDC | —d | — | T167T/I^{e} | — | — | — |
| | | AX4 | — | — | — | — | — | — |
| | | hCK | N296N/S^{f} | N296N/S^{f} | N296S | S153G | S153G | S153G |
| | BB131 | MDC | — | — | N446N/S | — | — | H411cH/ |
| | | AX4 | — | — | — | — | — | C53C/Yⁱ |
| | | hCK | — | — | — | — | — | — |
| | HP79 | MDC | — | — | — | — | — | — |
| | | AX4 | — | — | — | — | — | — |
| | | hCK | — | — | D27N | — | — | — |
| H3N2 | DA30^{j} | AX4 | — | N158N/K | N158K | — | — | — |
| | | hCK | — | — | — | — | — | — |
| | DA29-1^{j} | AX4 | — | — | — | — | T148K | T148K |
| | | hCK | — | — | D408D/N | — | — | — |
| | DA23-1^{j} | AX4 | — | — | — | — | — | — |
| | | hCK | — | — | — | — | — | — |
| B/Yamagata | HP70-2 | MDC | — | — | — | — | — | — |
| | | AX4 | — | — | — | — | — | — |
| | | hCK | — | S148S/N | S148N | — | — | — |
| | BB005 | MDC | — | — | — | — | — | — |
| | | AX4 | — | — | — | — | — | — |
| | | hCK | — | — | — | — | — | — |
| | DA09-2 | MDC | — | — | — | — | — | — |
| | | AX4 | — | — | — | — | — | — |
| | | hCK | — | — | — | — | — | — |
| B/Victoria | HP015 | MDC | — | — | — | — | — | D459D/N |
| | | AX4 | — | — | — | G208G/R | G208G/R | G208G/R |
| | | hCK | — | — | — | — | — | — |
| | WD28 | MDC | — | — | N196N/S | — | — | — |
| | | AX4 | — | — | — | — | — | — |
| | | hCK | — | — | — | — | L72L/F^{q} | L72F |
| | DA25-2 | MDC | — | — | — | — | — | — |
| | | AX4 | — | — | — | — | — | — |
| | | hCK | — | — | — | — | — | — |

^{a}Influenza viruses isolated form the clinical specimens were passaged ten times in MDCK, AX4, or hCK cells. The sequences of the HA and NA genes of the viruses were determined after a single passage (P1), the sixth passage (P6), and tenth passage (P10).
^{b}Mutations of influenza A viruses are shown with H3 numbering.
^{c}All mutations are shown with N2 numbering.
d—, No mutation was detected compared to the sequences from the original clinical specimens.
^{e}T/I, mixture of threonine and isoleucine at position 167
^{f}N/S, mixture of asparagine and serine at position 296.
^{g}N/S, mixture of asparagine and serine at position 446.
^{h}H/Y, mixture of histidine and tyrosine at position 411c.
ⁱC/Y, mixture of cysteine and tyrosine at position 53.
^{j}Influenza viruses were not isolated from the clinical specimens in MDCK cells.
^{k}N/K, mixture of asparagine and lysine at position 158.
^{l}D/N, mixture of aspartic acid and asparagine at position 408.
^{m}S/N, mixture of serine and asparagine at position 148.
ⁿD/N, mixture of aspartic acid and asparagine at position 459.
^{o}G/R, mixture of glycine and arginine at position 208.
^{p}N/S, mixture of asparagine and serine at position 196.
^{q}L/F, mixture of leucine and phenylalanine at position 72.

Establishment of a stable cell line possessing mutations in its ST3Gal genes and expressing the ST6Gal-I and HAT genes. gRNA sequences each targeting the ST3Gal-I,-II,-III,-IV, V,-VI, and ST3Gal-II-like protein genetic loci were designed using the sgRNA Design Tool from the Michael Boutros lab (http://www.e-crisp.org/E-CRISP/). The oligo DNA for the gRNA was cloned into the Cas9/gRNA dual expression vector pSpCas9(BB)-2APuro(PX459), encoding puromycin resistance (addgene). The resulting constructs were designated PX459-ST3Gal-I, PX459-ST3Gal-II, PX459-ST3Gal-III, PX459-ST3Gal-IV, PX459-ST3Gal-V, PX459-ST3Gal-VI, and PX459-ST3Gal-II-like, which express gRNA targeting ST3Gal-I,-II,-III,-IV, V,-VI, and ST3Gal-II-like protein genes, respectively. Human ST6Gal-I genes were amplified by PCR from the pCAGGS-FLAG-PUR-ST6Gal-I plasmid (Hatakeyama et al., 2005) and were then digested with NotI and XhoI. The digested fragment was cloned between the NotI and XhoI sites of the eukaryotic expression vector pCAG-Bsd, which encodes blasticidin resistance (Wako). The resulting construct was designated pCAG-Bsd-ST6Gal-I, which expresses ST6Gal-I. All constructs were sequence verified by Sanger sequencing. Cycle sequencing was performed using BigDye Terminator version 3.1 Cycle Sequencing Kits (Thermo Fisher Scientific), and sequences were analyzed on an ABI Prism 3130xl Genetic Analyzer (Thermo Fisher Scientific).

Electroporation was performed using the AMAXA Nucleofector II machine (Lonza) according to the manufacturer's instructions. Briefly, 5×10⁵ MDCK cells were resuspended in 100 μL of the desired electroporation buffer and mixed with either 5 μg of Cas9/gRNA dual expression vectors (1 μg PX459-ST3Gal-I, 1 μg PX459-ST3Gal-II, 1 μg PX459-ST3Gal-III, 1 μg PX459-ST3Gal-IV, 1 μg PX459-ST3Gal-VI, and 1 μg PX459-ST3Gal-II-like) or 1.7 μg of PX459-ST3Gal-V and 1.7 μg of pCAG-Bsd-ST6Gal-I. The resuspended cells were transferred to cuvettes and immediately electroporated using the program A-024. The cells were cultured in the presence of 2 μg/mL puromycin or 10 ug/ml blasticidin in MEM supplemented with 5% NCS to select for transfected cells. Clones were isolated using cloning rings, dissociated using trypsin and EDTA, and expanded. Genomic DNA was isolated using a genome isolation kit (Promega) according to the manufacturer's instructions. The target region was amplified by PCR using primers surrounding each target site, and amplification products were cloned by using a Zero Blunt TOPO PCR Cloning Kit (Invitrogen). At least eight clones were randomly selected for each gene and the isolated plasmids were sequenced. The list of primers used is provided in Table 5.

Flow cytometric analysis. Cells were detached by incubation for 10 min in PBS containing 0.125% Trypsin-20 mM EDTA (Dojindo). After being washed with PBS, the cells were blocked with Carbo-Free Blocking Solution (Vector) at 4° C. for 15 minutes. The cells were incubated with either biotinylated MAL II, SNA, or SSA at 4° C. for 30 minutes. The cells were then rinsed with PBS before being incubated with Alexa 488-conjugated streptavidin for 30 minutes at 4° C. (Invitrogen). Fluorescence was measured using a FACS Calibur or a FACS Verse (Becton Dickinson) and analyzed using FlowJo software (Becton Dickinson).

To confirm sialic acid-specific lectin binding, cells were treated, before incubation with lectin, with Clostridium perflingense (Roche) for 1 h at 37° C. Lectins bound to cells were detected as described above.

Immunofluorescence staining. Cells grown in 24-well plates were incubated with a mouse monoclonal antibody, which recognizes Siaα2,3 Galβ1,4GlcNAc (HYB4: Wako) at 4° C. After incubation, the cells were fixed with 10% trichloroacetic acid for 10 minutes at −20° C. Cells were then washed with PBS and incubated for 30 minutes with Alexa 488-conjugated goat anti-mouse immunoglobulin G (lgG) (Invitrogen). Cell nuclei were counterstained with Hoechst 33342, trihydrochloride, trihydrate (Molecular Probes). The samples were examined by using Zeiss fluorescence microscopy (model Imager Z1; Carl Zeiss).

TABLE A
Sequence analysis of the CRISPR/Cas9 target sites in hCK cells^{a}.

| α2,3-sialyltransferase gene | Mutation type |
|---|---|
| ST3Gal-I | 2 nucleotide deletion |
| ST3Gal-II | 1 nucleotide insertion |
| ST3Gal-III | 1 nucleotide deletion, |
| | 1 nucleotide insertion |
| ST3Gal-IV | 1 nucleotide deletion, |
| | 236 nucleotide insertion |
| ST3Gal-V | 8 nucleotide deletion, |
| | 1 nucleotide deletion, |
| | 2 nucleotide deletion, |
| | 1 nucleotide insertion |
| ST3Gal-VI | 1 nucleotide deletion |
| ST3Gal-II like | 1 nucleotide deletion |

^{a}PCR products of each gene were cloned into blunt-end vectors and subjected to sequencing analysis.

TABLE B
Mutations in α2,3-sialyltransferase genes caused by each gRNA

| Target | |
|---|---|
| gene | Sequence |
| ST3Gal- | CCTCCTTCTTCCTGAATTACTCCCACACC |
| I | CCTCCTT--TTCTGAATTACTCCCACACC |
| | CCTCCT---TTCCTGAATTACTCCCACACC (SEQ ID NO: 20) |
| ST3Gal- | CTTTACCTACTCCCACCACAGCATGGCCA |
| II | CTTTACCTACTCCCACCACAAGCATGGCCA(SEQ ID NO: 21) |
| ST3Gal- | CTCCCCCGCGGCTGTGGCGGCCGCCCGCG |
| III | CTCCCCCGCGGGCTGTGGCGGCCGCCCGCG |
| | CTCCCCCGC-GCTGTGGCGGCCGCCCGCG(SEQ ID NO: 22) |
| ST3Gal- | TCCCCAGGAACCCTGCTACCCTCTGAGAA |
| IV | TCCCCAGGA-CCCTGCTACCCTCTGAGAA |
| | TCCCCAGGACGATGGTGGTGTCAAAGTACTTGAAGGCGGCAGGGGCT |
| | CCCAGATTGGTCAGGGTAAACAGGTGGATGATATTCTCGGCCTGCTCT |
| | CTGATGGGCTTATCCCGGTGCTTGTTGTAGGCGGACAGCACTTTGTCC |
| | AGATTAGCGTCGGCCAGGATCACTCTCTTGGAGAACTCGCTGATCTGC |
| | TCGATGATCTCGTCCAGGTAGTGCTTGTGCTGTTCCACAAACAGCTGTTTC |
| | ACCCTGCTACCCTCTGAGAA (SEQ ID NO: 23) |
| ST3Gal- | ATCGCTCAAGACCCTCTCTGCAATGGTAC |
| V | ATCGCTCAAGACCCTCTCTG--ATGGTAC |
| | ATCGCTCAAGAC--------CAATGGTAC |
| | ATCGCTCAAGACCCTCTCTGGCAATGGTAC |
| | ATCGCTCAAGACCCTCTCT-CAATGGTAC (SEQ ID NO: 24) |
| ST3Gal- | AGCGATAAGTTTGATCTGCCCTATGGGATA |
| VI | AGCGATAAGTTTGATCTGCC-TATGGGATA(SEQ ID NO: 25) |
| ST3Gal- | CGCCCCGTCCAAGGATTTGAGATGGATGT |
| II-like | GCCCCCGTC-AAGGATTTGAGATGGATGT (SEQ ID NO: 26) |

The sequence of sgRNA is shown by bold letters. The underlined sequence shows the PAM sequence.
¹The sequence matched some sequence ot the PX459 vector.

TABLE 2
Isolation of human influenza viruses from clinical specimens^{a}.

| | | Number of virus isolates recovered | | |
|---|---|---|---|---|
| | Total number | (isolation efficiency)^{b} | | |
| Virus type | of specimens | MDCK cells | AX4 cells | hCK cells |
| A/H1N1pdm | 30 | 30 (100%) | 30 (100%) | 30 (100%) |
| A/H3N2 | 30 | 25 (83%) | 28 (93%) | 30 (100%) |
| B | 30 | 30 (100%) | 30 (100%) | 30 (100%) |

^{a}Clinical specimens shown to be influenza virus-positive by real-time RT-PCR or rapid diagnostic kits were used for virus isolation.
^{b}Clinical specimens were inoculated into MDCK, AX4, and hCK cells. Cells were observed for the development of cytopathic effect (CPE) for 7 days. Supernatants from CPE-negative cell culture samples were tested by using rapid diagnostic kits and hemagglutination assays with guinea pig red blood cells at 7 days after inoculation.

TABLE 3

| | | | Amino acid substitutions | |
|---|---|---|---|---|
| Sample ID | Virus | Cell | HA | NA |
| I-1202 | A/Yokohama/146/2017 | MDCK | —^{b} | D399G |
| | | AX4 | — | — |
| | | hCK | — | — |
| I-1205 | A/Yokohama/147/2017 | MDCK | — | D151N |
| | | AX4 | — | — |
| | | hCK | — | — |
| I-1218 | A/Yokohama/160/2017 | MDCK | — | — |
| | | AX4 | — | — |
| | | hCK | — | — |
| I-1221 | A/Yokohama/181/2017 | MDCK | — | D151D/G^{c} |
| | | AX4 | — | — |
| | | hCK | — | — |
| P-9211 | A/Yokohama/199/2017 | MDCK | N158K | R394K/R^{d} |
| | | AX4 | — | — |
| | | hCK | — | — |
| I-1250 | A/Yokohama/240/2017 | MDCK | — | — |
| | | AX4 | — | — |
| | | hCK | — | — |
| I-1244 | A/Yokohama/1/2018 | MDCK | — | — |
| | | AX4 | — | — |
| | | hCK | — | — |
| I-1248 | A/Yokohama/8/2018 | MDCK | — | D151D/N^{e} |
| | | AX4 | — | — |
| | | hCK | — | — |
| P-9256 | A/Yokohama/10/2018 | MDCK | P221S/P^{f}, | — |
| | | | N246D/N^{g} | |
| | | AX4 | — | — |
| | | hCK | — | — |
| P-9265 | A/Yokohama/14/2018 | MDCK | T248I | — |
| | | AX4 | — | D151D/N^{e} |
| | | hCK | — | — |
| P-9279 | A/Yokohama/15/2018 | MDCK | T160K/I^{h} | — |
| | | AX4 | — | — |
| | | hCK | — | — |
| P-9281 | A/Yokohama/16/2018 | MDCK | — | — |
| | | AX4 | — | — |
| | | hCK | — | — |
| P-9288 | A/Yokohama/20/2018 | MDCK | T160K/Tⁱ | D151D/N^{e} |
| | | AX4 | — | — |
| | | hCK | — | — |
| P-9291 | A/Yokohama/21/2018 | MDCK | — | D151D/N^{e} |
| | | AX4 | — | — |
| | | hCK | — | — |
| P-9301 | A/Yokohama/29/2018 | MDCK | — | — |
| | | AX4 | — | T148K/T^{j}, |
| | | | | D151D/N^{e} |
| | | hCK | — | — |
| I-1271 | A/Yokohama/33/2018^{k} | hCK | — | — |
| I-1275 | A/Yokohama/32/2018 | MDCK | — | — |
| | | AX4 | — | — |
| | | hCK | — | — |
| P-9307 | A/Yokohama/34/2018 | MDCK | — | — |
| | | AX4 | — | — |
| | | hCK | — | — |
| P-9315 | A/Yokohama/36/2018^{l} | AX4 | — | — |
| | | hCK | — | — |
| I-1279 | A/Yokohama/37/2018^{l} | AX4 | — | — |
| | | hCK | — | — |
| I-1280 | A/Yokohama/38/2018^{l} | AX4 | — | — |
| | | hCK | — | — |
| P-9328 | A/Yokohama/40/2018 | MDCK | — | — |
| | | AX4 | — | T148T/I^{m} |
| | | hCK | — | — |
| I-1288 | A/Yokohama/41/2018 | MDCK | — | — |
| | | AX4 | — | — |
| | | hCK | — | — |
| P-9330 | A/Yokohama/43/2018 | MDCK | — | D151D/G^{c} |
| | | AX4 | — | E433E/Kⁿ |
| | | hCK | — | — |
| P-9333 | A/Yokohama/44/2018 | MDCK | — | D151D/N^{e} |
| | | AX4 | — | D151D/N^{e} |
| | | hCK | — | — |
| P-9334 | A/Yokohama/45/2018 | MDCK | — | — |
| | | AX4 | — | D151D/G^{c} |
| | | hCK | — | — |
| P-9352 | A/Yokohama/48/2018 | MDCK | — | — |
| | | AX4 | — | — |
| | | hCK | — | — |
| P-9356 | A/Yokohama/49/2018^{k} | hCK | — | S44S/P^{o} |
| I-1295 | A/Yokohama/50/2018 | MDCK | T160K | — |
| | | AX4 | — | D151D/N^{e} |
| | | hCK | — | — |
| I-1296 | A/Yokohama/51/2018 | MDCK | N158K | — |
| | | AX4 | — | — |
| | | hCK | — | — |

^{a}Influenza viruses isolated from the clinical specimens in MDCK, AX4, or hCK cells. The sequences of the HA and NA genes of the viruses were determined after isolation in MDCK, AX4, or hCK cells.
^{b}—, No mutation was detected compared to the sequences from the original clinical specimens.
^{c}D/G, mixture of aspartic acid and glycine at position 151.
^{d}K/R, mixture of Lysine and arginine at position 394.
^{e}D/N, mixture of aspartic acid and asparagine at position 151.
^{f}S/P, mixture of serine and proline at position 221.
^{g}D/N, mixture of aspartic acid and asparagine at position 246.
^{h}K/I, mixture of lysine and isoleucine at position 160.
ⁱK/T, mixture of lysine and threonine at position 160.
^{j}K/T, mixture of lysine and threonine at position 148.
^{k}Influenza viruses were not isolated from the clinical specimens in MDCK and AX4 cells.
^{l}Influenza viruses were not isolated from the clinical specimens in MDCK cells.
^{m}T/I, mixture of threonine and isoleucine at position 148.
ⁿE/K, mixture of glutamic acid and lysine at position 433.
^{o}S/P, mixture of serine and proline at position 44.

TABLE 4
Comparison of the sensitivity of hCK and
AX4 cells to human influenza viruses^{a}.

| | | | Highest dilution | | |
|---|---|---|---|---|---|
| | | | of clinical sample | | |
| | | | showing CPE | | Ration (hCK |
| | | | observed in^{b} | | highest dilu- |
| | Influenza | Sample | AX4 | hCK | tion/AX4 high- |
| Virus type | season | ID | cells | cells | est dilution |
| A/ | 2017-18 | BB139 | 8192 | 32768 | 4 |
| H1N1pdm | | UT001-1 | 16 | 512 | 32 |
| | | HP79 | 65536 | 16384 | 0.25 |
| | 2016-17 | P-8848 | 8192 | 8192 | 1 |
| | | BB131 | 524288 | 524288 | 1 |
| | 2013-14 | IMS1 | 8192 | 16384 | 2 |
| A/H3N2 | 2017-18 | DA29-1 | <2 | 64 | >64 |
| | | DA30 | <2 | 2048 | >2048 |
| | | HP62 | 32 | 4096 | 128 |
| | 2016-17 | DA23-1 | 16 | 2048 | 128 |
| | | DA16-2 | <2 | 16 | >16 |
| | | DA19-2 | 256 | 2048 | 8 |
| B/ | 2017-18 | BB140 | 16384 | 16384 | 1 |
| Yamagata | | HP70-2 | 512 | 4096 | 8 |
| | | BB152 | 262144 | 262144 | 1 |
| | 2016-17 | BB005 | 128 | 256 | 2 |
| | 2015-16 | DA09-2 | 65536 | 524288 | 8 |
| | | DA07-2 | 64 | 64 | 1 |
| B/ | 2017-18 | WD28 | 16384 | 131072 | 8 |
| Victoria | 2016-17 | DA25-2 | 8192 | 32768 | 4 |
| | | BB078 | 524288 | 524288 | 1 |
| | | BB130 | 4096 | 4096 | 1 |
| | 2015-16 | HP015 | 2048 | 16384 | 8 |
| | | HP009 | 262144 | 262144 | 1 |

^{a}Clinical specimens shown to be influenza virus-positive by real-time RT-PCR were used for virus isolation.
^{b}Serial 2-fold dilutions (2¹ to 2²⁰) of clinical samples were prepared and inoculated into AX4 and hCK cells. Cells were observed for the development of CPE for 7 days. Three wells were used to infect with the same dilutions of virus, and the highest dilution showing CPE in all three wells is shown.

TABLE 5
List of primers used.

| Primer or | | | |
|---|---|---|---|
| probe | Target gene | Sequence (5′-3′)^{a} | Orientation |
| ST3Gal- | Canis lupus | CCCTCCTCGTCCTCTTCATC (SEQ | Forward |
| I-F | familiaris | ID NO: 27) | |
| | ST3Gal-I | | |
| ST3Gal- | Canis lupus | AGGCAGAGAGAGACCAGAGA | Reverse |
| I-R | familiaris | (SEQ ID NO: 28) | |
| | ST3Gal-I | | |
| ST3Gal- | Canis lupus | CCAAACCATGAAGTGCTCCC | Forward |
| II-F | familiaris | (SEQ ID NO: 29) | |
| | ST3Gal-II | | |
| ST3Gal- | Canis lupus | AGGGGCTTGAAGAGTGACTC | Reverse |
| II-R | familiaris | (SEQ ID NO: 30) | |
| | ST3Gal-II | | |
| ST3Gal- | Canis lupus | ATGAGACTTGCTTGCATCCC (SEQ | Forward |
| III-F | familiaris | ID NO: 31) | |
| | ST3Gal-III | | |
| ST3Gal- | Canis lupus | CTTTGGTTGGCCTCTCTGTCTC | Reverse |
| III-R | familiaris | (SEQ ID NO: 32) | |
| | ST3Gal-III | | |
| ST3Gal- | Canis lupus | CGTTAGCCGCGCGCACAG (SEQ ID | Reverse |
| III-seq-R | familiaris | NO: 33) | |
| | ST3Gal-III | | |
| ST3Gal- | Canis lupus | CCGGGATGACAGCTCTC (SEQ ID | Forward |
| IV-F | familiaris | NO: 34) | |
| | ST3Gal-IV | | |
| ST3Gal- | Canis lupus | ACATGGAAGCTGGACTCAC (SEQ | Reverse |
| IV-R | familiaris | ID NO: 35) | |
| | ST3Gal-IV | | |
| ST3Gal- | Canis lupus | CATCATCACAAGGATCCTGC (SEQ | Forward |
| V-F | familiaris | ID NO: 36) | |
| | ST3Gal-V | | |
| ST3Gal- | Canis lupus | CTCTCCCATGAAAACCTGG (SEQ ID | Reverse |
| V-R | familiaris | NO: 37) | |
| | ST3Gal-V | | |
| ST3Gal- | Canis lupus | GTTTTAAATTTGGGAGCGGCC | Forward |
| VI-F | familiaris | (SEQ ID NO: 38) | |
| | ST3Gal-VI | | |
| ST3Gal- | Canis lupus | TGGCTCACATCAAACACCAC (SEQ | Reverse |
| VI-R | familiaris | ID NO: 39) | |
| | ST3Gal-VI | | |
| ST3Gal- | Canis lupus | GGTTGGAAACTCAAGGTGCC (SEQ | Forward |
| II-like-F | familiaris | ID NO: 40) | |
| | ST3Gal-II-like | | |
| ST3Gal- | Canis lupus | TGACTCCTTCCCCTTTTCCC (SEQ | Reverse |
| II-like-R | familiaris | ID NO: 41) | |
| | ST3Gal-II-like | | |
| RT/PCR- | A/H1N1pdm | GTTACGCGCCAGCAAAAGCAGGG | Forward |
| A/H1N1 | virus HA | GAAAACAAAAGCAA (SEQ ID | |
| pdm-HA- | | NO: 42) | |
| F | | | |
| RT/PCR- | A/H1N1pdm | GTTACGCGCCAGTAGAAACAAGGG | Reverse |
| A/H1N1 | virus HA | TGTTTTTCTCATGC (SEQ ID NO: 43) | |
| pdm-HA- | | | |
| R | | | |
| RT/PCR- | A/H1N1pdm | GTTACGCGCCAGCAAAAGCAGGA | Forward |
| A/H1N1 | virus NA | GTTTAAAAT (SEQ ID NO: 44) | |
| pdm-NA- | | | |
| F | | | |
| RT/PCR- | A/H1N1pdm | GTTACGCGCCAGTAGAAACAAGGA | Reverse |
| A/H1N1 | virus NA | GTTTTTTGAACAAC (SEQ ID NO: 45) | |
| pdm-NA- | | | |
| R | | | |
| RT/PCR- | A/H3N2 virus | GTTACGCGCCAGCAAAAGCAGGG | Forward |
| A/H3N2- | HA | GATAATTCTATTAA (SEQ ID NO: 46) | |
| HA-F | | | |
| RT/PCR- | A/H3N2 virus | GTTACGCGCCAGTAGAAACAAGGG | Reverse |
| A/H3N2- | HA | TGTTTTTTAATTAATG (SEQ ID | |
| HA-R | | NO: 47) | |
| RT/PCR- | A/H3N2 virus | GTTACGCGCCAGCAAAAGCAGGA | Forward |
| A/H3N2- | NA | GTAAAGATG (SEQ ID NO: 48) | |
| NA-F | | | |
| RT/PCR- | A/H3N2 virus | GTTACGCGCCAGTAGAAACAAGGA | Reverse |
| A/H3N2- | NA | GTTTTTTCTAAAATTGC(SEQ ID | |
| NA-R | | NO: 49) | |
| RT/PCR- | Influenza B | GTTACGCGCCAGCAGAAGCAGAGC | Forward |
| IBV-HA- | virus HA | ATTTTCTAATATCC (SEQ ID NO: 50) | |
| F | | | |
| RT/PCR- | Influenza B | GTTACGCGCCAGTAGTAACAAGAG | Reverse |
| IBV-HA- | virus HA | CATTTTTCAATAACGTTTC (SEQ ID | |
| R | | NO: 51) | |
| RT/PCR- | Influenza B | GTTACGCGCCAGCAGAAGCAGAGC | Forward |
| IBV-NA- | virus NA | ATCTTCTCAAAACTG (SEQ ID | |
| F | | NO: 52) | |
| RT/PCR- | Influenza B | GTTACGCGCCAGTAGTAACAAGAG | Reverse |
| IBV-NA- | virus NA | CATTTTTCAGAAAC (SEQ ID NO: 53) | |
| R | | | |
| qPCR- | A/H1N1pdm | AGAAAAGAATGTAACAGTAACAC | Forward |
| A/H1N1 | virus HA | ACTCTGT (SEQ ID NO: 54) | |
| pdm-F | | | |
| qPCR- | A/H1N1pdm | TGTTTCCACAATGTARGACCAT | Reverse |
| A/H1N1 | virus HA | (SEQ ID NO: 55) | |
| pdm-R | | | |
| qPCR- | A/H3N2 virus | CTATTGGACAATAGTAAAACCGGG | Forward |
| A/H3N2- | HA | RGA (SEQ ID NO: 56) | |
| F | | | |
| qPCR- | A/H3N2 virus | GTCATTGGGRATGCTTCCATTTGG | Reverse |
| A/H3N2- | HA | (SEQ ID NO: 57) | |
| R | | | |
| qPCR- | B/Victoria | CCTGTTACATCTGGGTGCTTTCCTA | Forward |
| B/Victoria- | virus HA | TAATG (SEQ ID NO: 59) | |
| HA-F | | | |
| qPCR- | B/Victoria | GTTGATARCCTGATATGTTCGTATC | Reverse |
| B/Victoria- | virus HA | CTCKG (SEQ ID NO: 60) | |
| HA-R | | | |
| qPCR- | B/Yamagata | CCTGTTACATCCGGGTGCTTYCCTA | Forward |
| B/Yamagata- | virus HA | TAATG (SEQ ID NO: 61) | |
| HA-F | | | |
| qPCR- | B/Yamagata | GTTGATAACCTKATMTTTTCATAT | Reverse |
| B/Yamagata- | virus HA | CCTCTG (SEQ ID NO: 62) | |
| HA- | | | |
| R | | | |
| MP-39- | Type A virus | CCMAGGTCGAAACGTAYGTTCTCT | Forward |
| 67 For | M | CTATC (SEQ ID NO: 63) | |
| MP-183- | Type A virus | TGACAGRATYGGTCTTGTCTTTAG | Reverse |
| 153 Rev | M | CCAYTCCA (SEQ ID NO: 64) | |
| NIID- | Type B virus | GGAGCAACCAATGCCAC(SEQ ID | Forward |
| TypeB | NS | NO: 65) | |
| TM | | | |
| Primer- | | | |
| F1 | | | |
| NIID- | Type B virus | GTKTAGGCGGTCTTGACCAG(SEQ | Reverse |
| TypeB | NS | ID NO: 66) | |
| TM | | | |
| Primer- | | | |
| R1 | | | |
| FAM- | A/H1N1pdm | (FAM)CAGCCAGCAATRTTRCATTT | |
| A/H1N1 | virus HA | ACC(BHQ-1) (SEQ ID NO: 67) | |
| pdm-HA- | | | |
| Probe | | | |
| NIID- | A/H1N1pdm | (FAM)CAGCCAGCAATRTTRCATTT | |
| swH1 | virus HA | ACC(MGB/TAMRA) (SEQ ID NO: 68) | |
| Probe2 | | | |
| HEX- | A/H3N2 virus | (HEX)AAGTAACCCCKAGGAGCAAT | |
| A/H3N2- | HA | TAG(BHQ-1) (SEQ ID NO: 69) | |
| HA- | | | |
| Probe | | | |
| NIID-H3 | A/H3N2 virus | (FAM)AAGTAACCCCKAGGAGCAA | |
| Probe1 | HA | TTAG(MGB/TAMRA) (SEQ ID NO: 70) | |
| FAM- | B/Victoria | (FAM)TTAGACAGCTGCCTAACC(BHQ-1) | |
| B/Victoria- | virus HA | (SEQ ID NO: 71) | |
| HA- | | | |
| Probe | | | |
| FAM- | Victoria | (FAM)TTAGACAGCTGCCTAACC | |
| Type B | B/Victoria | (MGB/TAMRA) (SEQ ID NO: 72) | |
| HA | virus HA | | |
| HEX- | B/Yamagata | (HEX)TCAGGCAACTASCCAATC | |
| B/Yamagata- | virus HA | (BHQ-1) (SEQ ID NO: 73) | |
| HA- | | | |
| Probe | | | |
| FAM- | Yamagata | (FAM)TCAGGCAACTASCCAATC | |
| Type B | B/Yamagata | (MGB/TAMRA) (SEQ ID NO: 74) | |
| HA | virus HA | | |
| MP-96- | As Type A | (FAM)ATYTCGGCTTTGAGGGGGCC | |
| 75 Probe | virus M | TG(MGB/TAMRA) (SEQ ID NO: 75) | |
| NIID- | Type B virus | (FAM)ATAAACTTTGAAGCAGGAAT | |
| TypeB | NS | (MGB/TAMRA) (SEQ ID NO: 76) | |
| Probe1 | | | |

aFAM, 6-carboxyfluorescein; HEX, hexacholoro-6-carboxyfluorescein; BHQ-1, black hole quencher; MGB, minor groove binder; TAMRA, 6-carboxytetramethylrhodamine.

### Results

A new MDCK cell line (designated hCK) was prepared that overexpresses α2,6 -sialoglycans and expresses extremely low levels of α2,3 -sialoglycans to mimic the sialic acid expression pattern of human upper respiratory epithelial cells (see FIGS. 2-4 and Table A).

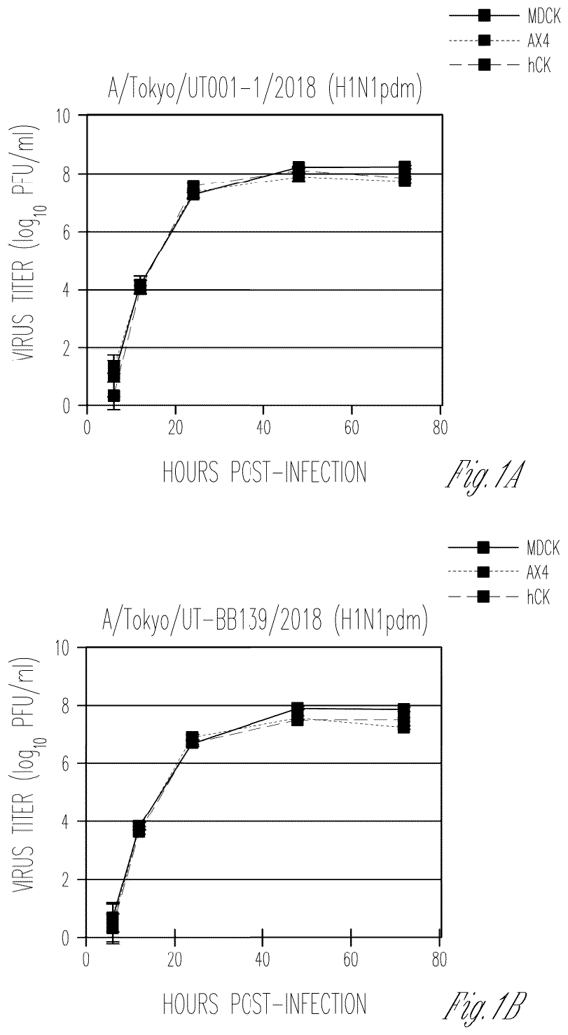

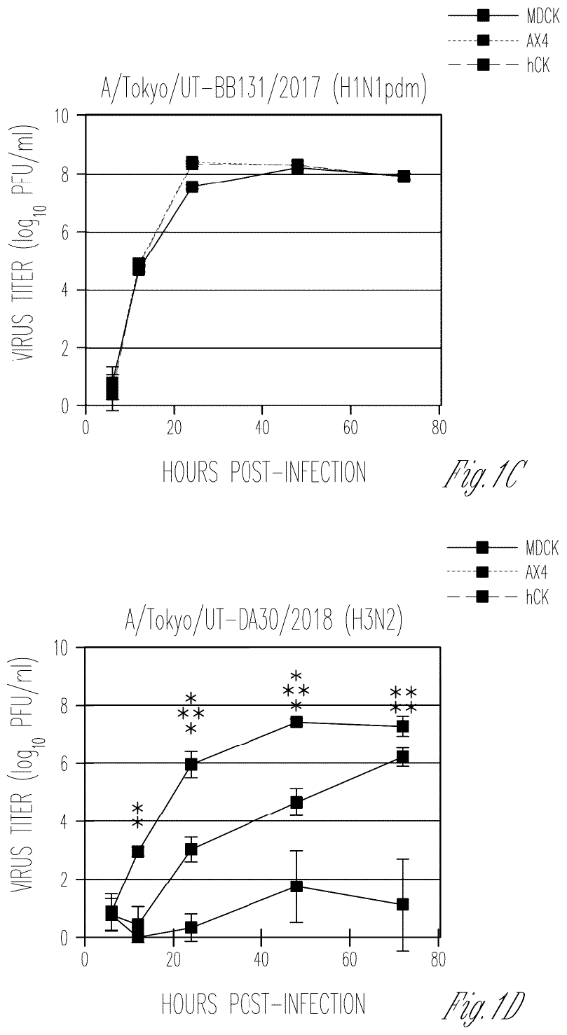

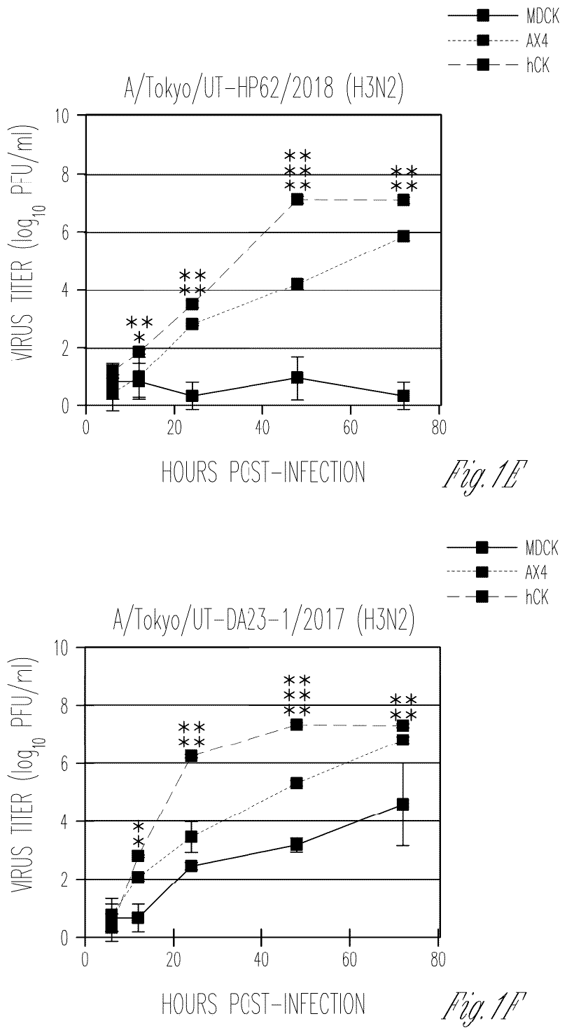

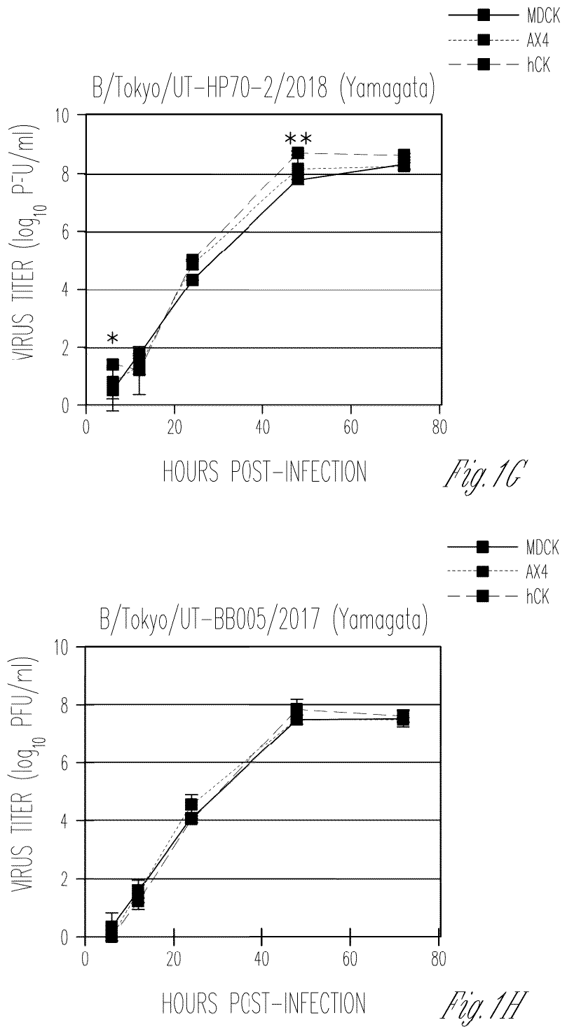

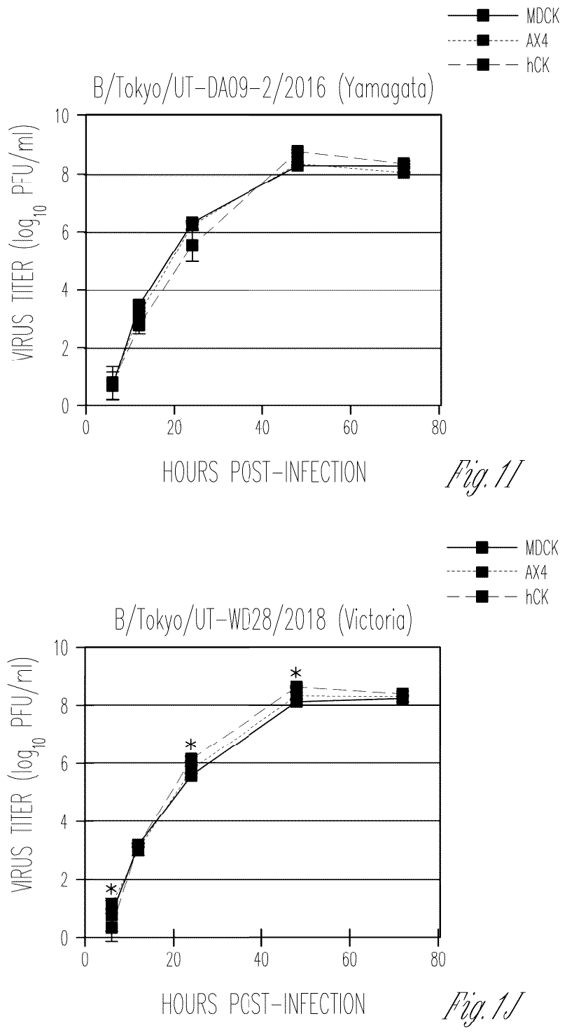

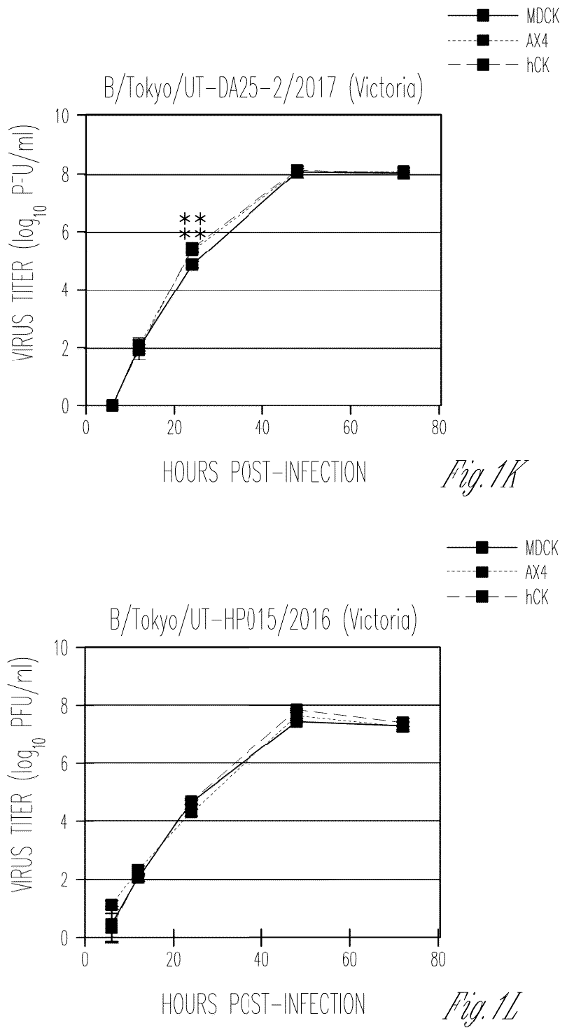

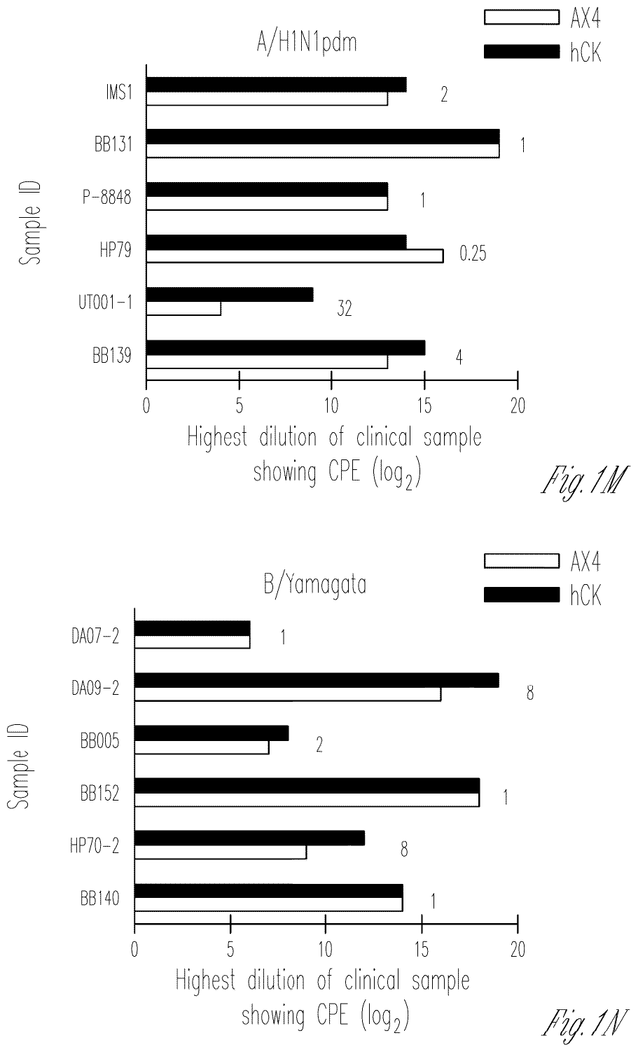

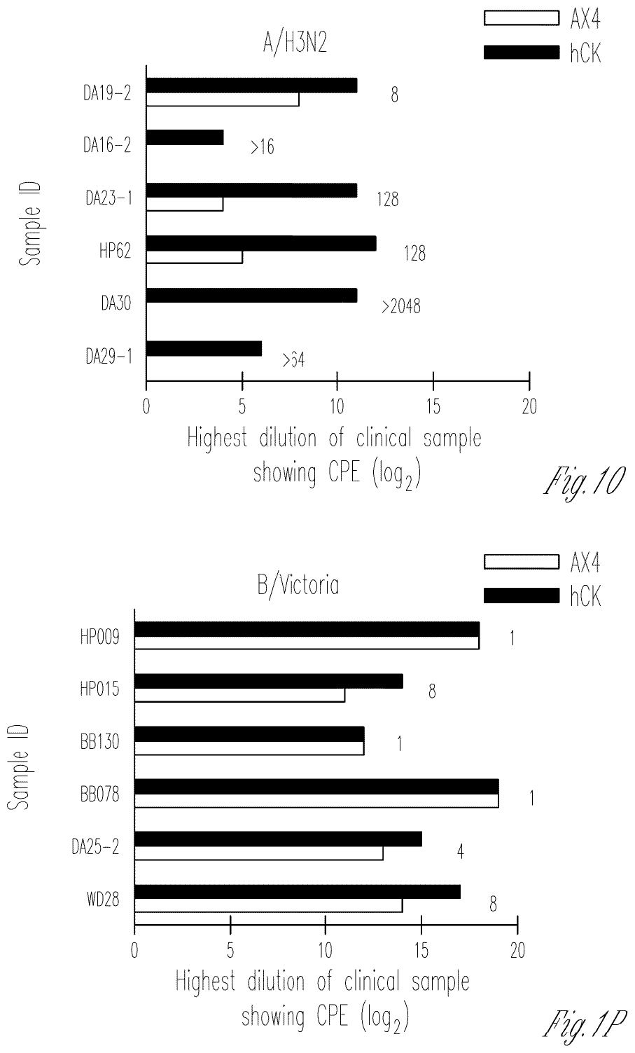

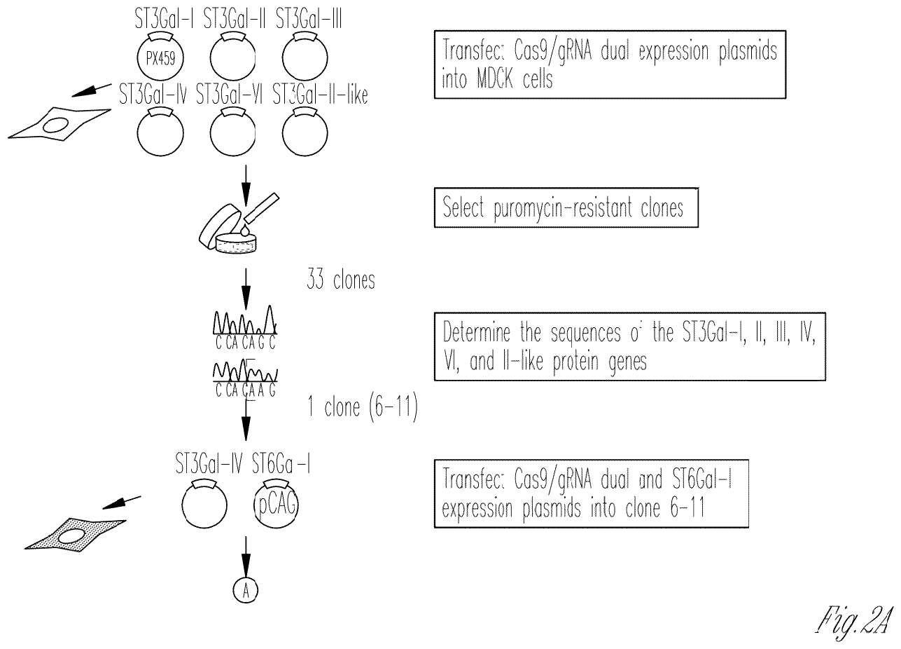

To determine whether hCK cells could support efficient replication of human influenza viruses, the growth kinetics of viruses [3 A/H1N1 2009 pandemic (A/H1N1pdm), 3 A/H3N2, 3 B/Yamagatalineage, and 3 B/Victoria-lineage] were examined in hCK cells. The three A/H1N1pdm isolates grew efficiently in MDCK, AX4, and hCK cells, and no substantial differences in titers were observed (FIG. 1A). The six influenza B isolates also replicated with similar efficiency in all three cell lines. By contrast, for A/H3N2 viruses, all three isolates grew much faster and to higher titers (2.03 to 2.91 log units higher at 48 h post-infection) in hCK cells than in AX4 cells. As reported elsewhere (Chambers et al., 2014), in MDCK cells, these recent A/H3N2 isolates replicate poorly. These findings demonstrate that hCK cells, which express very low levels of α2,3 -sialoglycans and high levels of α2,6 -sialoglycans, more efficiently support the replication of recent A/H3N2 viruses than do either MDCK or AX4 cells.

To evaluate the susceptibility of hCK cells for isolation of human influenza viruses, aliquots of 90 respiratory specimens (30 A/H1N1pdm, 30 A/H3N2, and 30 B/Yamagata-lineage) were inoculated into MDCK, AX4, and hCK cells. The cells were observed for the development of cytopathic effect (CPE) for 7 days. For MDCK, AX4, and hCK cells, A/H1N1pdm viruses were successfully recovered from all of the RT-PCR-positive samples without the need for blind passages (100% isolation efficiency) (Table 2). Similarly, these three cell lines showed 100% efficiency for the isolation of influenza B viruses. For the A/H3N2-positive samples, 5 and 2 viruses were not recovered from MDCK and AX4 cells, respectively. These results are consistent with previous reports (Oh et al., 2008; Hatakeyama et al., 2005) that conventional MDCK cells have relatively low sensitivity for the detection of recent A/H3N2 viruses.

The agglutination of red blood cells by influenza viruses is thought to be due to the virus binding to sialic acids on the surface of the cell. Since 2005, A/H3N2 isolates have lost their ability to agglutinate turkey red blood cells (Lin et al., 2013). In addition, current A/H3N2 isolates show reduced or no agglutination of guinea pig red blood cells (Lin et al., Influenza Other Respir Viruses, 2017), indicating a change in their avidity for sialic acid receptors. Indeed, Lin et al. (2013) measured the avidity of recent A/H3N2 viruses for α2,6 -linked sialic acid receptors and showed that it has decreased drastically. Glycan array analysis has revealed that recent A/H3N2 isolates prefer binding to branched sialylated N-linked glycans with extended poly-N acetyllactosamine chains (Peng et al., Cell Host Microbe., 2017).

By contrast, virus isolation from hCK cells was successful with all samples without any subsequent blind passage, suggesting that this cell line is more effective than AX4 or MDCK cells for the isolation of human A/H3N2 viruses from clinical specimens.

During replication of recent A/H3N2 human isolates in MDCK cells, the viruses rapidly acquired amino acid changes at positions 148 and 151 of the NA protein (e.g., T148I and D151G), which affect the biological properties of NA. To examine whether the A/H3N2 viruses isolated from the three cell lines possessed mutations in their HA and NA proteins, the nucleotide sequences of the HA and NA segments of the isolates were determined by means of Sanger sequencing (Table 3). Sequence analysis revealed that 7 out of 25 MDCK-grown isolates contained an amino acid change at position 151 of NA compared with the sequence from the original specimens: NA-151N, NA-151D/G, and NA-151D/N (mixed populations of amino acids at position 151). Amino acid changes leading to the loss of the glycosylation site at position 158 of HA were found among virus populations of some other MDCK-grown isolates: HA-158K, HA-160K, HA-160K/I, and HA-160K/T. These changes are known to alter the antigenic properties of HA (Lin et al., 2017; Chambers et al., 2015; Skowronski et al., 2016). Importantly, cell culture-adaptive mutations were also found in the NA protein of several isolates propagated in AX4 cells: NA-148K/T, NA-148T/I, NA-151D/N, and NA-151D/G. Strikingly, no mutations were detected in hCK-grown isolates, except for only one isolate that possessed an S44P mutation in its NA stalk. These findings strongly suggest that hCK cells support the efficient growth of A/H3N2 viruses without accompanying cell culture-adaptive mutations.

Seasonal influenza viruses from clinical specimens grow better in AX4 cells than in MDCK cells (Hatakeyama et al., 2005). To determine whether hCK cells are superior to AX4 cells for virus isolation, the sensitivity of hCK and AX4 cells were compared by testing serial 2-fold dilutions of specimens. Aliquots of 24 specimens (6 A/H1N1pdm, 6 A/H3N2, 6 B/Yamagata-lineage, and 6 B/Victoria-lineage) were inoculated into AX4 and hCK cells in triplicate. All culture wells were examined for CPE on day 7 post-inoculation, and the ratios of the highest dilutions showing CPE observed in hCK cells to those in AX4 cells were determined. For one of the six A/H1N1pdm-positive samples (sample ID, HP79), hCK cells were slightly less sensitive than AX4 cells (FIG. 1B and Table 4). For the remaining samples, however, the sensitivity of hCK cells was similar to or greater than that of AX4 cells. For the B/Yamagata- and B/Victoria-lineage-positive samples, hCK cells showed sensitivities equal to or somewhat greater than that of AX4 cells For all of the A/H3N2-positive samples, hCK cells showed greater sensitivity than AX4 cells; for some samples, hCK cells were approximately 100-to 2,000-fold more sensitive than AX4 cells. Taken together, these results indicate that hCK cells are more suitable than AX4 or MDCK cells for the primary isolation of recent seasonal A/H3N2 viruses.

To evaluate the genetic stability of the HA and NA genes of viruses isolated in hCK cells, aliquots of 12 clinical specimens (3 A/H1N1pdm, 3 A/H3N2, and 3 B/Yamagata-lineage, and 3 B/Victoria-lineage) were inoculated into MDCK, AX4, and hCK cells, and the isolates were sequentially passaged ten times. After the first, sixth, and tenth passages, the HA and NA sequences of the viruses were determined by Sanger sequencing, and the sequences were compared to those in the clinical specimens. For A/H1N1pdm-positive specimens, a mixed viral population encoding either N or S at position 296 of HA was detected in one out of the three hCK-grown viruses (BB139) after the first passage (Table 1). The hCK-grown virus also possessed an S153G substitution mutation in its NA. Another hCK-grown virus (HP79) encoded a D27N substitution in its HA after the tenth passage. A mixed population encoding either T or I at position 167 of HA was found in one MDCK-grown virus after the tenth passage (BB139). Another MDCK-grown virus (BB131) had a mixed population encoding HA-446N and HA-446S at passage ten. The MDCK-grown virus also contained a mixed population encoding either H or Y at position 411c of NA. A mixture of C53Y/C in NA was observed in one AX4-grown virus after the tenth passage (BB131).

For A/H3N2-positive samples, viruses that were recovered from AX4 and hCK, but not MDCK, cells were serially passaged. After the sixth passage, a mixed population encoding HA-158N and HA-158K (leading to the loss of the glycosylation site at position 158 of HA) was detected in one of the three AX4-grown viruses (DA30). In addition, another AX4-grown virus (DA29-1) encoded a T148K substitution in its NA after the sixth passage. A mixed population encoding HA-408D and HA-408N was detected in one hCK-grown virus after the tenth passage (DA29-1).

For B/Yamagata-lineage viruses, no changes were detected in any isolates after the first, sixth, or tenth passages, with the exception of a mixed population encoding HA-148S and HA-148N detected in one hCK-grown virus at passage six (HP70-2). For B/Victoria lineage viruses, a mixed population encoding NA-208G and NA-208R was found in one of the three AX4-grown viruses (BB139) after the first passage. After the sixth passage, one hCK-grown viruses encoded a mixture of L72L/F in its NA (WD28). At passage ten, one MDCK-grown virus (WD28) contained an N196S mutation known to lead to the loss of the glycosylation site at position 196 of HA (B/Victoria-lineage), which can significantly alter the antigenicity of influenza B viruses. Another MDCK-grown virus (HP015) had a mixture of D459D/N in its NA.

Overall, A/H1N1pdm and B viruses were slightly more variable when passaged in MDCK or hCK cells than in AX4 cells. In contrast, A/H3N2 viruses propagated in hCK cells maintained higher genetic stability than those in AX4 cells.

In conclusion, a cell line derived from MDCK cells, hCK, expresses large amounts of α2,6 -sialoglycans and small amounts of α2,3 -sialoglycans that will be useful for influenza virus research, particularly studies involving human A/H3N2 influenza viruses and possibly for vaccine production.

### REFERENCES

Bates et al., J. Stat. Softw., 61:1 (2015).
Chambers et al., Cell Rep., 12:1 (2015).
Chambers et al., J. Viral, 88:10986 (2014).
Chu and Whittaker, Proc. Natl. Acad. Sci. U.S.A., 101:18153 (2004).
Cong et al., Science, 339:819 (2013).
Connor et al., Virology, 205:17 (1994).
Gamblin and Skehel, J. Biol. Chem., 285:28403 (2010).
Han et al., Cell Rep., 23:596 (2018).
Hatakeyama et al., J. Clin. Microbiol. 43:4139 (2005).
Hegde, Hum. Vaccine Immunother., 11:1223 (2015).
Hidari et al., Biochem. Biophys. Res. Commun., 436:394 (2013).
Jinek et al., Science, 337:816 (2012).
Lee et al., PLOS One 8 (2013).
Lenth, J. Stat. Softw. 69 (2016)
Li et al., J. Clin. Microbial., 47:466 (2009).
Lin et al., Influenza Other Respir Viruses, 11:263 (2017).
Lin et al., J. Viral., 84:6769 (2010).
Lin et al., PLOS One, 12, 72299 (2017).
Matrosovich et al., Journal of Virology, 77:8418 (2003).
Mohr et al., Viral J., 12:67 (2015).
Oh et al., J. Clin. Microbial. 46:2189 (2008).
Rogers and Paulson, Virology, 127:361 (1983).
Shalem et al., Science, 343:84 (2014).
Shibuya et al., J Biochem., 106:1098 (1989).
Shinya et al., Nature, 440:435 (2006).
Skowronski et al., Euro. Surveill, 21:30112 (2016).
Stevens et al., J. Mal. Biol., 355:1143 (2006).
Takashima and Tsuji, Trends in Glycoscience and Glycotechnology, 23:178 (2011).
Tamura et al., Antimicrob. Agents Chemother., 57:6141 (2013).
van Riel et al., Science, 312:399 (2006).
Zhu et al., J. Viral, 86:13371 (2012).

All publications, patents and patent applications are incorporated berein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. An isolated recombinant mammalian cell, comprising a reduced amount of cell surface β-galactoside α2,3 sialyl residues relative to a corresponding non-recombinant mammalian cell, wherein all β-galactoside α2,3 sialyltransferase (ST3Gal) genes are mutated so as to reduce the amount of the cell surface β-galactoside α2,3 sialyl residues, and wherein the recombinant mammalian cell comprises an expression cassette encoding human β-galactoside α2,6 sialyltransferase (ST6Gal), wherein the reduced amount of cell surface 3-galactoside α2,3 sialyl residues is the result of mutation of the ST3Gal genes in the recombinant mammalian cell.

2. The isolated recombinant cell of claim 1 further comprising an increased amount of human β-galactoside α2,6 sialyl residues relative to a corresponding non-recombinant mammalian cell.

3. The isolated recombinant cell of claim 1 which comprises an expression cassette encoding human β-galactoside α2,6 sialyltransferase I (ST6Gal-I) or II (ST6Gal-II).

4. A method of modifying the amount of cell surface β-galactoside α2,3 sialyl residues and human β-galactoside α2,6 sialyl residues on a mammalian cell, comprising:
mutating all β-galactoside α2,3 sialyltransferase (ST3Gal) genes, and overexpressing a human β-galactoside α2,6 sialyltransferase (ST6Gal) gene, in a parental mammalian cell so as to result in a modified mammalian cell having a reduced amount of cell surface β-galactoside α2,3 sialyl residues and an increased amount of human β-galactoside α2,6 sialyl residues on the surface of the modified cell relative to the corresponding parental mammalian cell.

5. The method of claim 4, wherein the mutations include one or more nucleotide insertions or one or more nucleotide deletions, or both, in the ST3 genes.

6. The method of claim 4 wherein the modified cell comprises an expression cassette comprising a ST6Gal open reading frame.

7. A method of detecting or propagating an influenza virus, comprising:
infecting the recombinant cell of claim 2 with a sample having or suspected of having an influenza virus.

8. The method of claim 7 further comprising collecting progeny virus.

9. The method of claim 7, wherein the sample is from an avian or a mammal suspected of being infected with an influenza virus.

10. The method of claim 7, wherein the influenza virus is a human influenza virus.

11. The method of claim 7, wherein the influenza virus is an influenza A or B virus.

12. The method of claim 7, wherein the influenza virus is a H3 virus.

13. The method of claim 7, wherein the influenza virus is A/H1N1, A/H3N2, a B/Yamagata-lineage influenza B virus or a B/Victoria-lineage influenza B virus.

14. The method of claim 7 further comprising detecting whether the sample is infected with an influenza virus.

15. The method of claim 14 further comprising identifying the HA and/or NA subtype of the virus.

16. The isolated recombinant mammalian cell of claim 1 which is infected with human influenza virus.

17. The isolated recombinant mammalian cell of claim 1, wherein the mammalian cell is a canine, nonhuman primate cell or a rodent cell.

18. The isolated recombinant mammalian cell of claim 17, wherein the nonhuman primate cell is a chimpanzee, ape or monkey cell.

19. The isolated recombinant mammalian cell of claim 17, wherein the rodent cell is a as mouse, rats, rat and guinea pig cell.

20. The isolated recombinant mammalian cell of claim 1, wherein the ST3Gal genes include one or more of ST3Gal-I, ST3Gal-II, ST3Gal-III, ST3Gal-IV, ST3Gal-V, ST3Gal-VI, or ST3Gal-II-like genes.


SEQUENCE LISTING (151 sequences)

SEQ ID NO: 1 - protein, 406 aa, Homo sapiens
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 2 - protein, 529 aa, Homo sapiens
MKPHLKQWRQRMLFGIFAWGLLFLLIFIYFTDSNPAEPVPSSLSFLETRRLLPVQGKQRAIMGAAHEPSPPGGLDARQALPRAHPAGSFHAGPGDLQKWAQSQDGFEHKEFFSSQVGRKSQSAFYPEDDDYFFAAGQPGWHSHTQGTLGFPSPGEPGPREGAFPAAQVQRRRVKKRHRRQRRSHVLEEGDDGDRLYSSMSRAFLYRLWKGNVSSKMLNPRLQKAMKDYLTANKHGVRFRGKREAGLSRAQLLCQLRSRARVRTLDGTEAPFSALGWRRLVPAVPLSQLHPRGLRSCAVVMSAGAILNSSLGEEIDSHDAVLRFNSAPTRGYEKDVGNKTTIRIINSQILTNPSHHFIDSSLYKDVILVAWDPAPYSANLNLWYKKPDYNLFTPYIQHRQRNPNQPFYILHPKFIWQLWDIIQENTKEKIQPNPPSSGFIGILIMMSMCREVHVYEYIPSVRQTELCHYHELYYDAACTLGAYHPLLYEKLLVQRLNMGTQGDLHRKGKVVLPGFQAVHCPAPSPVIPHS

SEQ ID NO: 3 - protein, 353 aa, Homo sapiens
AAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 4 - protein, 529 aa, Homo sapiens; identical to SEQ ID NO: 2

SEQ ID NO: 5 - DNA, 6830 bp, Canis lupus
  source 1..6830: mol_type = other DNA
gtcaggtctcagaaaagtctagaataaggcttacaggcactttgttcagttgtggaacacatggaaacttcatacaccgcccccctccttgcagaccggagagctctctgctctaatttgggcacaggcgcccaaccttgggccccagaggaagcctgttctactcccagggccaacgctcaattgcttggtaacttggcacccacactgttccaggctctggtctcagtttcactctctgcaaaatgagaggctcagagttccgctgacatggcctcggggccgttaaaacctttcttcatgaagacagcctgcacccttcctgcttctgctccaggtccatcctcagaaccttccagaaagtcctggcacgtcagataacagggccaacccggcagtgatgccccaccccccaccccttcccttactcatagagcctgctccagactctcagagcccacacccacttggtgaagtcatttgccagtaattcttcgcactggacattgagaggtttcagaccccagaagtctcaggcgctgggtctgaaagtgggcagagcccaggtgacatttgtggagactctcagtggtgcgtatagccgccgggaccattttcagactcaacctttctgacctggaaatgccaatagaagtaatcatcatcgccaggggctgtggtgagcaaatgcttggagctctgtggccagggcagtttcatttgaggaccagagatggacaatccctcagcctactgagatgaagaaactgagtctcagagaggttaaggaactcccccgaggttgcaccactgaggaagattgacctgacttcccaagaccatacatctggtaaaccggaacctgcacctgcgccatctcaagcctactctggaggcccgaggctaattggcagagtttgagggctgagatgacagacaaccctcagtgccttcatcgggcgggctgcttacctgcacatccctggtgacagcatgggaaagaccgcttctaattaagcgtcatcacacatcacccttttccggaggaagagaggcaaagacagctcccctctatcctgcactgtgaaccttcctccgaggtcctcccctcacccccgagagtccttgccctgtcaccaagattaattacccctcaacccctttggatggcaaaggcagtcattttattaagtttgattaaagcttcaagagacattgccggatgtttcaggactgctgacaaagcagcctgcttgtttcctgaaaagaccaattatatgcaagaagcgtcagccccactccccgaggggtccacttagcctccgaccaccacagggtgatgtccagcgcaccggtgtggccatcacctggcgggagtgaggtcggccgtagcaggtctggaggcccccctgcaagtccctgcctcaccctgtgactgagcctctcgtctctgggaattttgttccaaattccccgtctaccaggtgtgatttcctccagccccaccagccctgggaggcgcccatccagagagcagagatggtgaccatgagaaagaggactctcaaagtgctcaccctcctcgtcctcttcatcttcctcacctccttcttcctgaattactcccacaccatggtgaccaccacctggtttcccaagcagatggttgtcgagctctcagagaactttaagaagttcatgaaatacactcacaggccttgcacctgtgcccgctgcatcgggcagcagagggtctcggcctggttcgatgagaggttcaaccggtccatgcagccgctgctgaccgcccagaacgccctcttggaggaggacacctacagctggtggctgaggctccagcgggagaagcaacccaacaacttgaacgataccatcagggagctgttccaggtggtgcccgggaacgtggaccccctgctggagaagaggtcggtgggctgccggcgctgcgcagtcgtgggcaactctggcaacctccgagagtcctggtacgggcctcagatcgacagccacgacttcgtgctcaggatgaacaaggcccccacggcgggcttcgagatggatgtcgggagcaagaccacccaccacctggtgtaccccgagagcttcagggagctggcggagaatgtcagcatggtcctggtgcccttcaagaccaccgacctggagtgggtggtcagtgccaccaccacaggcaccatctctcacacctatgttcctgttcctgcaaagatcaaagtgaaaaaggataagatcctcatctaccacccggccttcatcaagtacgtcttcgacagctggctgcagggccacgggcggtacccgtccaccggcatcctctccgtcatcttctcgctgcacatctgcgacgaggtggacttgtacggcttcggggcagacagtaaggggaactggcatcactactgggagaacaatccatcggcgggggctttccgcaagaccggggtgcacgacggagactttgagtccaacgtgacggccaccttggcgtccatcaataagatccggatatttaaggggagatgacgctgccgaggagcaccggagcccgcctctttggccagccccagcctctgctggagccgatctgtgctgggggctttgagggccagcctcgggggcgtgttcaggtgcccctcgtgccccctcgcaccccgacatttggcagcatcgactcagcaaggccccagagccccgctgggtctgcagagcgagtgtcagaactggtcttggatggggacaccccccctccacctccctgcaccgccactgctgtcctggagcagggggcaggtggggagacgcgggaggaggtccggtctccaaacgctcaagtcatttcggctttggggggcagagggggagatatcgaagactctgtgttctaaagtgaaggacactggccctgggcagaggggtcccgggctgactctctgactctgatgcttgctgggtgaggacgacctcgggacagagccacatcgggccacgtggacgctgggggtgagaaggcaccttcctctgcacgatgcccggcccctccctcaccgcctctgcggtcttcccgaagctcctccgtggcggccaggagaggcgccctgcgccgagggtcctcacagatgcttggccaagtgtttcagactccagcaggtgtcttcttcgcaagctgaggctcccttgagtgatcgatctttgtggccataaatgatggctgagagcagatctgactacttccacgtgcctttgtgtctgggggagagctgtgcgcattggctgaaataaggcaaaagccttaattcgggagtggggagctcccccctctccccgcccccagcaatgccacccccttgctctggagctgggtaacatctttactagtttcctgaggcggtgccggagctggaatgaagctaggatgatgctcaacggcgtccagaggtgcgtcagccgcggcacgtcattcttaggttcacagactgatggatttggggcccgtgaccttccaaggccacacacagaacctccaccagccagcactttgccccaggagcccaccctccaggccaagtccctccgggccacctgcacagtgtcctggggtttgtggggccatcccaggaacatgaccctcggagcgggagaagacatacctcatcctctcacttctgggctgctctgattcctcctcatgattatttattgatttttttttaattcagctgacattgcgtgcactttgtggctccgtggctggtcctgatgttttaattaagcttgctctcgcttcacctggcagccggggcatggggggcttaagtcaaggtttgcaggagtcctcagacttgggagggggatgcatatctagggcttgggggctcgtcggtgggcaaatgccagggtttcaggttggtaggttctccgagtgctctgtacctccttcccctcagctctgcctcccttctccatcattgcgggttgggatcattctcaggctaagaatctgcaaaacccagtgagacggccttggggccaggtggggagtggagtcacacaggcagggcagcgatggttggctccggttgctgacactgaatcagaaaatccacgtttcctattgagagcatttcctaacaggccactgctacttcgaggaggtgtgacagtgtcctggctgtcacgggggcccgtctcctgtgctccttgcagcagcactccggcaagttctgctctcctggcttcagtctctgcctctgcaaacggagagggaggcatttggggagctcagggggccacaggtggtttcagggggttcacaagcgtccccagagagagcacaccacgcccgtgttttaccccaagcctgtgtgtatgagcatatattcctggggagcggtgccgtagcgttcattcaattgtcagaggttcagaacccagacaatggtttacaaaaacacgaaacgacagcaagcaaacaaaaacccgttcgagctttaaaatcctccaacacttagactctcttgacccctaaaggctcataaatcactacgaggtcagcaagctgatcataaatcccaaagactatatagcaggcatctgtgggttggctgatgaatccgctgagagaagtgggtgcggagcggcctccgagctcctccccgagtcctggaagagccgagtccagtcagagctgatgggtccagagcgctcagcctgcgtcggggctgtgctgagtcaggacaggtccccagagtaagggggggggtctctctccctccccaccccactgagccgtccaggctgttgagaggacttcccacctgccttcccatgtaacacttgagaaaagcgaggcccggagaagggaagtggcttgcaaagtgaacccgtggtgctggacaacgcaagtcagaccctttgagcaaagatagagcacgattgctacatgttgaacacacctcgaggtgggctttcccagccggcttccatccagcctccccaaggactgtgaccgggcacaggtgggctcctcaggctgcacgtggtggcccccgcaagctccggtgtctgccttttccccccatccctccagtgagggagacacccttgctcttccctcagccccagcaaacttgtctttgtgttcgttggcttagcaaaggggtctgacccgaatgcaccaacgggcttcagccccctggcatctgtacctggggtaagggtggctgagaatccaaccagagtatttcccccagagctcgtggctgtgggcaggttaacgagaaccacccgccccccccactcggaggaggctcctgaatgcccaccccagtgttgggctcccagcctagggccctgcttgctcctcaagcacagagctcccttggaggccagtcctggacccctgggcagggagcagaggcatccctgcctctctcagtgctgagcccgaagccaggaagaccagggccgaggagggagagagagattttaacaccctccccggccctggggagccaggagctggagtcacctgccgcaaatgcaaggcctggttccacgtgcccccctccgcccctgtcctcacccccttgttagggtgggtttgtagcgtctggctcccagagccatcaggcaaaaaagaaagttcttaaagagatgcttttaggggcctgtgcttagtgggagaagccagattcaagaaaatgggacttaaaacggggaagccagacgagaaggtggggggggggttgtccgggtgtgattcttacaagtgctgaggccagaaggatggaccctctccaggtccagagtagagtggagtcaacggggaatatttaggactgtgatgtttgcagtaatggtcctcttcaaggcagcctggacacaaagcaaaatcggaaacagaaaagaactcaccatcccttgcgtgggtaggaccaaaacagctttctccagggaggcgcaagccctgagtggccgccgtctggccaaagccggcctcgtgccatcgggaaccggccaccgcccatgcccctccggagagctcggggtcgtcatcgtgtccaccacgggcatctggtcggaagcaaacgttcccacaggccactggctggtcatcgtcccctgtggtctgggctgaccggtttgagaccaagtccgctgccttgctccggcctgtcctgaagacaccagcgccccggcctgtggggtgccccgtgatgcctccatgcagggaagagcaggggaccagggaggaagagcagagacaggtgaagcgacagtccccgcgtcccagcctcagcattcgcatcctcttggcccctacttttcctctccgcccagcagacatctgccctgcccttgcccttgaccccattgctgcgcttccctcaaggacgggcctggccttggtggccacctgcggacagccctgcgcccgacgcccgcttcaccccggggcccgggtctggaggggccgcccccaggacgaacgcggctgccccacggggccggcccctcaccggcttcgcgtccaagccaaagtttctcgagcacttttttgttctttgcaatcatgttgggttcattgttggtgttttaaaattttgcttccctctccctctggcctcgctcctgtgtgtgttttgtagccgagcgctaacctggatgcttttttgaatgacctttgcaagagcctgccttcctcggcctctgctctgttttatttattgttgaatatttccaatgatccaaatcaaagtgaattaaaacaaagctattttatcgtt

SEQ ID NO: 6 - protein, 342 aa, Canis lupus
MVTMRKRTLKVLTLLVLFIFLTSFFLNYSHTMVTTTWFPKQMVVELSENFKKFMKYTHRPCTCARCIGQQRVSAWFDERFNRSMQPLLTAQNALLEEDTYSWWLRLQREKQPNNLNDTIRELFQVVPGNVDPLLEKRSVGCRRCAVVGNSGNLRESWYGPQIDSHDFVLRMNKAPTAGFEMDVGSKTTHHLVYPESFRELAENVSMVLVPFKTTDLEWVVSATTTGTISHTYVPVPAKIKVKKDKILIYHPAFIKYVFDSWLQGHGRYPSTGILSVIFSLHICDEVDLYGFGADSKGNWHHYWENNPSAGAFRKTGVHDGDFESNVTATLASINKIRIFKGR

SEQ ID NO: 7 - DNA, 4577 bp, Canis lupus
  source 1..4577: mol_type = other DNA
cggctcgctccgggagggcagagccggcaagggcgggactggcctgctgcgggcggacgggggagccgcggagctaacgggtccggacgtcgccagcggcggagctttctgcacgggcggaggcggcggcaggagcggcaagcatggcccgcgcggcgcccggcgctgactgagggtcatgctgcggcagcctggcgcctgacgagtgagggggaacctgccaggggattactgctagcacgagctaacagcaggacagcaggcctcaggggagtggggatgggcatagcagcctgcctgcaacgctgggccagtggcaccaaggaggcccaggccctggcagggagctcggcacacgctcagctgacccagacagcattcaaagcaggtccctgctgagcagcagctgttcccaggccctggaggaccgcaaacgcctcagcaaccacatttggggctgtgaccatcaggagaagggaccaacacctcgaatgggaagcagaagggcagaacctgctgccaggaggatcttggtctctgcagcaaagtcttccgacacccgtggagcttcttgggtttctcctctcaggactggggccttgtttcctcctctggtggagggggtaggccaggagggccagctgaggtggggacgactctcggatggaatccaggccacttgaagcccatgggctctctgctgtgattggggttcgagggcttccccactgcaggggacgaggggccgcctccgtgtctgctcatgaaccacaaggaccccgaatgctccagactggaccatttcgagccaccaaagagtggggcccccagagctggcagccagcagctccaagggactagagagctgggatggactgacctccccctcaccagggcattaggagagtcagagcctctgtggcccagctagtgacagagagacccgatgaggccataagctggcacccgagtgactcagggacaggagagccactcctgcctactgtgctttctctttacattgagggagggtgtggtaagggacccctgcctctgttccctcctcagcacgtgcccctatgccctttgcacgtggtgccaggagacgcaggctacactgtggctggcccatcagtgggctgggaagggagtggccacggtgacacccacccgcccaccaggctggtggtctagccccccggggcgccaaaccatgaagtgctccctgcgggtgtggttcctctctgtggccttcctgctggtgttcatcatgtcactgctctttacctactcccaccacagcatggccaccttgccctacctggactcaggggccctgggcggtacccaccgggtgaagctggtgcctggctatgctggtctgcagcgcctcagcaaggaggggctcaccggtaagagctgtgcctgccgccgctgcatgggtgacactggcgcctctgactggtttgacagccacttcaacagcaacatttcccctgtgtggacccgagagaatatggatctgcctccagatgtccagaggtggtggatgatgctgcagccccagttcaagtcacacaacaccaacgaggtactggagaagctgttccagatagtaccaggcgagaacccctaccgtttccgggacccccaccagtgccggcgctgtgcggtagtggggaactcaggcaacctgcggggctctggctacggcccagatgtggatgggcataacttcatcatgaggatgaatcaggcgccaaccgtgggctttgagcaggatgttggcagccgaactacccaccatttcatgtaccccgagagtgccaagaaccttcctgccaacgtcagctttgtgttggtgcccttcaaagctctggacctactatggattgccagcgctttgtccacagggcaaatcagattcacctatgcgccagtgaagtccttccttcgagtggacaaagaaaaggttcagatctacaacccggcattcttcaagtacatccacgaccggtggacagagcatcacgggcggtacccttccacagggatgctggtgctcttctttgccctgcatgtttgtgatgaggtgaacgtgtacgggttcggggccgacagccggggcaactggcaccactactgggagaataaccggtatgcgggcgagttccggaagacgggagtgcacgacgccgacttcgaggcccacatcatcgacatgctggccaaggccagcaagatcgaggtctaccgaggcaactgagcccggcctcgccgcgacccttccggcccagccgcgaggctgcgacgctcgctccgcagccgggactcccggccagcccgagggcggcggccttggcgagggcgcggcgcccggttgggcgtctccagccctgggagcgacgccaagtcgcggtctggaccaatcatgctgcaagtccagcgagcgccggctgtcccccgccaatcaggagactttgggggctggcccaggcctggcacccaatcagcgctgcagtgggagcggaggctctttctcccagccaatcatgcgactcaaggagaacttccggcgctgggcccggtctcctccaatcaatggccttcggaggcgggccggccgccgctgaatccccactcccctatgctttgggtaggattttattttatgctttttaaggagtagtggttggttccggcctcagtggagtactttcctcaggctctgcgggaggagtgttggtggcctgtcggcggtactcggccaggggcaccgaggaggaagcgggggggaaggtgcggggcagcagcggctgggcctcccttggccgggggcccctcgcgacctcggggcgggcgggggggcgggggcgttggcctcccgcttctggaggtccggggggaatcaggtggtttccggggagcgcgctttcatccccgggaagagctagatccctctcaaaccttttcaggcctcagagcactctagaccgcgtatttcctttatctgtcgggcccagatgggtgagtgtaaacccaccaaagaaaggcagtggagcgtgggtcccctcgtcctcccttgtccccatccccacctttggccaccctatgggatggccttcctgaccagggcattgagcatcccacctggaaactagaactgtattcacccgttgctagggctcggagttcgccgatggcctaactcgagcgcaggggtaaggaacagggcaggggacccggaagccccgtcacttcagatgtaaggtgcttctcactctcgtgtactctcgcggccccttactgttcgcccacaacttttttagtgtcccttcttaagccctgggcctcctcaccagcctgctcgtcctgggattgggggtggggggtggggcactgctggcttctccaaccccctacccctcctcgctcgtctttagccggctctagggagaggaaggcaggctggagatggggagcccagctgcctggtgcatgcaccgttttcctccgcccatcaccccaaagaggagtaggaaacctcttgcttgggggtggaatttgctttggtctcctaatttagttaacttgaggttaccagggatggctgaccaacaaagattcttttaaaattccaggctggccatgcaaattgctgggatcctagctggggaggagtcgactgacttgcccgccttgcatgtctcctctcctgcccctgcgtcccctccctctgccacgctcacttcctgcctcatctctccaacccattttccattttcagctctagaagggcagggacgcttacaaacaggagttacatctggaagttacttccaagactgaacccagcttaagtccctagaggaagctgctgatgatattctcacccttcaaggttggggaagtttcggagggggaaagtgcttctgtgaagcttccaaaccactaataggatcccccttcccaacaatgaggaacacaaacaccaccctttatcttagttgataccaccaagcagcctcctggccattggggtaattcctgcagctggctggggtaaccagcaggggagtatattagaggaggattggggcagggcagtgggcacccctaaagttaatatattgagaacttagcttaagcctaagtcttagttccttcccaattccaaaagtaggaggagcaacgagtgggggtggatttgggggggcctatcctggaatgcctctctcagggcttcccccaccattttagagagtcaaggcaccagccattcatgccagtctcctctcagtgcttcctgaagaggctgtttggagtgttcggaaaatgaaaaaaacaatgcaattatgccaaacagtattgagcagaataatttatttcttttttgttcctttttcttctttttgttttgtttaaaacattaataaatcccctttctggaagaggtaggtcccagcatccagcccagatctccttttctgcaatagttatttaaacaaatgtttgtttgtttttttattttcttccctttctctctctttctgaattaaaaaaaaagaaaactccta

SEQ ID NO: 8 - protein, 350 aa, Canis lupus
MKCSLRVWFLSVAFLLVFIMSLLFTYSHHSMATLPYLDSGALGGTHRVKLVPGYAGLQRLSKEGLTGKSCACRRCMGDTGASDWFDSHFNSNISPVWTRENMDLPPDVQRWWMMLQPQFKSHNTNEVLEKLFQIVPGENPYRFRDPHQCRRCAVVGNSGNLRGSGYGPDVDGHNFIMRMNQAPTVGFEQDVGSRTTHHFMYPESAKNLPANVSFVLVPFKALDLLWIASALSTGQIRFTYAPVKSFLRVDKEKVQIYNPAFFKYIHDRWTEHHGRYPSTGMLVLFFALHVCDEVNVYGFGADSRGNWHHYWENNRYAGEFRKTGVHDADFEAHIIDMLAKASKIEVYRGN

SEQ ID NO: 9 - DNA, 1782 bp, Canis lupus
  source 1..1782: mol_type = other DNA
ttggtggtcgcgctccgcccgccgctgcgtccccaccatggcggcgcccgtgcagcccaccgcgtcgtgggcgcccgccgggctcccccgcggctgtggcggccgcccgcgcctcggcctccgcctccccgcccgcgccggcccgggcgccgcctccccgctgcctccgtctccgctgcggtcatgtaggaaatcgtaaatcatgtgaagatgggactcttggtatttgtacgcaatctgctgctagccctctgcctttttctggtactgggatttttgtattattctgcgtggaagctgcatttactccagtgggaggactccaattcagtggttctttcctttgactccgctggacaaacactaggctcagagtatgatcggttgggtttcctcctgaagctggactctaaactgcctgctgagttagccaccaagtatgcaaacttttcagagggagcttgcaagcctggctatgcttcggccttgatgactgccatctttccccggttctccaagccagcacccatgttcctggatgactctttccgcaagtgggctaggattcgggagtttgtgccgccttttgggatcaaaggtcaagacaatctgatcaaagccatcttgtcagtcaccaaagagtaccgcctgacccctgccttggacagcctcagctgccgccgctgcatcatcgtgggcaacggaggtgtcctagccaacaagtctctggggtcacgaattgatgactatgacattgtggtcagactgaactccgcaccagtgaaaggctttgagaaggacgtgggcagcaaaactacactgcgcatcacctaccctgagggcgccatgcagcggcctgagcaatatgaacgcgattctctatttgtcctcgctggcttcaagtggcaggacttcaagtggttgaagtacatcgtctacaaggagagagtgctctgggcccgcagggatacctgccaatctgtctgggcccatccccctctcccctccaccagctgtcaccagccaccccaggggaggggtcctgcagagttcaggccattcttcttccaatacccgagcctcctactggaggagaatgatgacagacagcctctggcgacaagtgcatcagatggcttctggaaatccgtggccacacgagtgcccaaggagccccctgagattcgcatcctcaacccgtacttcatccaggaggccgccttcaccctcatcggactgcccttcaacaacggcctcatgggccgcgggaacatcccgacccttggcagtgtggcagtgaccatggcgctacacggctgtgatgaggtggcagtcgcaggctttggctacgacatgagcacacccaacgcgcccctgcactactatgagaccgtgcgcatggcagccatcaaagaggtcaccagcgactcagctcaaggctgccaaatccagtggacacatggaagcctcatctttcctgacctcccagaaatgctttttctgttgaccactccttcctctttgaaacttttcctgctcagactgtcctggacacacaatatccagcgagagaaagagtttctgcgcaagctggtgaaggcgcgcgtcatcaccgacctaaccagcggcatctgaggtgggcccagcacatggccacggaggtcctggcaccgccaagaggaagccgcagccactgccacctgtccacttcattggcctcggtctggctctgcctgaaaggcgcaggagtcttcagacccagagaaggacagtgccaagggg

SEQ ID NO: 10 - protein, 474 aa, Canis lupus
MGLLVFVRNLLLALCLFLVLGFLYYSAWKLHLLQWEDSNSVVLSFDSAGQTLGSEYDRLGFLLKLDSKLPAELATKYANFSEGACKPGYASALMTAIFPRFSKPAPMFLDDSFRKWARIREFVPPFGIKGQDNLIKAILSVTKEYRLTPALDSLSCRRCIIVGNGGVLANKSLGSRIDDYDIVVRLNSAPVKGFEKDVGSKTTLRITYPEGAMQRPEQYERDSLFVLAGFKWQDFKWLKYIVYKERVLWARRDTCQSVWAHPPLPSTSCHQPPQGRGPAEFRPFFFQYPSLLLEENDDRQPLATSASDGFWKSVATRVPKEPPEIRILNPYFIQEAAFTLIGLPFNNGLMGRGNIPTLGSVAVTMALHGCDEVAVAGFGYDMSTPNAPLHYYETVRMAAIKEVTSDSAQGCQIQWTHGSLIFPDLPEMLFLLTTPSSLKLFLLRLSWTHNIQREKEFLRKLVKARVITDLTSGI

SEQ ID NO: 11 - DNA, 1819 bp, Canis lupus
  source 1..1819: mol_type = other DNA
ccctacaggcccgagctgccggggtcgggcctccccgggttcccgctcccgggtcctcctggacacaccggcctggcctggctcccggggaactctcgtctgctagcggggagcctccctccgcctcgcccacgggcacccctcccacccagtatccttggcctcttgcaggtggcccgaggcagccgggatgacagctctccccaggaaccctgctaccctctgagaaacatgatcagcaagtcccgctggaagctcctggccatgttggctctggtcctggtcgtcatggtgtggtattccatctcccgagaagacaggtacattgagcttttttattttcccatcccagagaagaaggaaccgtgcttccagggtgaggcagagagaaaggcctctaagctctttggcaactactcccgagatcagcccatcttcctgcagatgaaggattatttctgggtcaagacaccgtctgcctacgagctgccctatgggaccaaggggagcgaagacctgctcctccgggttctagccatcaccagctactccattccagagagcatccagagtctcaagtgtcgccgctgcgtggtggtgggcaatgggcatcggctgcgcaacagctcgctgggagatgccatcaacaagtacgacgtggtcatcagactgaacaacgcccccgtggctggctacgagggtgacgtgggctcgaagaccaccatgcgtctcttctacccggagtcagcccacttcaaccccaaagtggagaacaacccagacacacttctcgtcctggtggccttcaaggcaatggacttccactggattgagaccatcctgagtgataagaagagggtacgaaagggcttctggaagcagcctcccctcatctgggacgtcaaccccaggcaggttcggattctcaaccctttctttatggagattgcagctgacaaactgctgaacctgccaatgaaacagccacgcaagatttcccagaagcccaccacgggcctgctggccatcacgctggctctccacctctgcgacctggtgcacatcgccggcttcggctacccggacgcccacaacaggaagcagaccattcactactatgaacagatcacgctcaagtccatggcggggtcaggccacaacgtctcccaggaggccctggccatcaagcggatgctggagatcggagcagtcaagaacctcacgttcttctgacggggacaggagctctagccgtcagtctgcccgccctgccgcctgagcgaccaaccacggctgtgggggcgccggcgtgacctgcttggattccccctccccgtgtggagagggggcctggtacaggcggggcctgagatggggccgcgcccctggctgctcttggggcggccggatccagtcagggtggaggccccgggtggcgggaggccttccgaggcgcggggtgtgtggctgaggcaccccttctcaccagccccgggagcttatttaatgggctatttaattaaaagggtaggaatgtgcctcgggctggtcccatggcatccggaaacgggggcatagcacagtggtctgcccactgtggataaaaacacacaagtgcttggcccactagagcctagagccagagcaggcctcccaggagggcaggggcgtctggagcgggtgggtgccctccagagaggggctgctacctcccagcgggcatgggaagagcattgggatgaggtcccacggagaatgggacctcatgtagaaaagaggtttgaaacctaacattaaactattttttcctaaaacggaa

SEQ ID NO: 12 - protein, 333 aa, Canis lupus
MISKSRWKLLAMLALVLVVMVWYSISREDRYIELFYFPIPEKKEPCFQGEAERKASKLFGNYSRDQPIFLQMKDYFWVKTPSAYELPYGTKGSEDLLLRVLAITSYSIPESIQSLKCRRCVVVGNGHRLRNSSLGDAINKYDVVIRLNNAPVAGYEGDVGSKTTMRLFYPESAHFNPKVENNPDTLLVLVAFKAMDFHWIETILSDKKRVRKGFWKQPPLIWDVNPRQVRILNPFFMEIAADKLLNLPMKQPRKISQKPTTGLLAITLALHLCDLVHIAGFGYPDAHNRKQTIHYYEQITLKSMAGSGHNVSQEALAIKRMLEIGAVKNLTFF

SEQ ID NO: 13 - DNA, 1385 bp, Canis lupus
  source 1..1385: mol_type = other DNA
cgctctggaaccacttacagccacctggtgcatcctcctttggggtgcgtttggagggcctggttcctgctcagccacatcttctgccactttcaccagcaatgcccagtgagtataactatgtaaaactgagaagcgatcgctcaagaccctctctgcaatggtacacccgagctcaaaacaagatgagaagacccaacttgttgttaaaagacatccttaagtgtacattgcttgtgtttggagtgtggatcctttatattctcaagttaaattatactactgaagaatgtgacatgaaaaaaatgcattatgtggacccagaccgtgtaaagagagctcagaaatatgctcagcaagtcttgcaaaaggagtgccgacccaagtttgcgaagaagtcgatggcgcagttgttcgagcacaggtacagcacggacttgccacctttcgtgaaggagacccccaaaatgaatgaagccgagtacaagtatgatcctccttttggattccgaaagttctccagtgaagtccagaccctgttggaaatactgcccgagcatgacatgcccgaacacttgagagcaaagagctgtaggcgttgtgtggtcatcggaagcggtggcatactccacggactagcactgggccaggccctcaaccagttcgatgtggtgataaggttaaacagtgcaccagttgagggatattctgagcatgttggtaataaaactactataaggatgacttatccagagggcgcgccactgtctgaccttgaatattattccaatgacttgtttgttgctgttttattcaagagtgttgacttcaactggcttcaagcaatggtaaaaaatgaaaccctgccattttgggtgcggctcttcttttggaagcaggtggcggaaaaaatcccactacagccaaaacatttcaggattttgaatccagttattatcaaagagactgcctttgacatccttcaatactcagagccccagtcaaggttctggggccgagataagaacgtgcccaccattggtgtcattgccgttgtcttagccacacatctgtgtgatgaagtcagcttggcaggctttggatatgacctcaatcaacccaaaacacctttgcactactttgacaatctctgcatggctgccatgaactttcaaaccatgcataatgtgacaacggagaccaggttcctcctcaagctggtcaaagagggcgtggtgaaggatctcagcggaggcatccattgtgaattttgaacacagggaaacctcatgtgacaatgcaactctgactctgaaggctgtttttcgtagccttctcgatgcagcgcatcctgcaaaatacttagaggtgcagctggggttttt

SEQ ID NO: 14 - protein, 390 aa, Canis lupus
MPSEYNYVKLRSDRSRPSLQWYTRAQNKMRRPNLLLKDILKCTLLVFGVWILYILKLNYTTEECDMKKMHYVDPDRVKRAQKYAQQVLQKECRPKFAKKSMAQLFEHRYSTDLPPFVKETPKMNEAEYKYDPPFGFRKFSSEVQTLLEILPEHDMPEHLRAKSCRRCVVIGSGGILHGLALGQALNQFDVVIRLNSAPVEGYSEHVGNKTTIRMTYPEGAPLSDLEYYSNDLFVAVLFKSVDFNWLQAMVKNETLPFWVRLFFWKQVAEKIPLQPKHFRILNPVIIKETAFDILQYSEPQSRFWGRDKNVPTIGVIAVVLATHLCDEVSLAGFGYDLNQPKTPLHYFDNLCMAAMNFQTMHNVTTETRFLLKLVKEGVVKDLSGGIHCEF

SEQ ID NO: 15 - DNA, 3159 bp, Canis lupus
  source 1..3159: mol_type = other DNA
ggtcgattgccccttggctgctgtggaggctgtgatgacctccagggccgcggccctccgggcgatgcttctccaggggctgaggccaacgcagaactcccgtggcacccactcggactcgcggcgtgttcacatgtggggttttattaaatcctcccaccaaccgtgtgagacaggaacagttagccccggtgtgtccgccaagattgccccgcacaagtggctccggatggatcacacgaagcacttgcaagtgaagaagcagcacagccctttatcttgggctatttcctgtggagagactccaacaatttagcagccaggctcctgggcctctgggaccctcaccacatcacatccttcaccttcaggagcagagcgcctttgggaaacagacttctaaaagtgcaggtgggccagccatgagagggtacctagtggccatattcctgagtgctgtctttctctattatgtgctgcattgtatattgtggggaacaaacatctattgggtgccacctgtggaaatgaagcggagaaataagatccagccttgtttagcgaagccagcttttgcctctctcctgaggtttcatcagtttcacccttttctgtgtgcagctgattttaaaaagattgcttccttgtatggtagcgataagtttgatctgccctatgggataagaacatcagcggaatattttcgactcgctctttcaaaactgcagagttgtgatctctttgatgagtttgacaatgtgccgtgtaaaaagtgcgtggtggttggtaatggaggagttctgaagaataagacattaggagaaaaaattgactcctatgatgtcataataagaatgaataatggtcctgttttaggacatgaagaggaagttgggagaaggacaaccttccgacttttttatccagaatctgttttttcagatcccaatcacaatgatcctaatactacagcgattctcactgcttttaagccgcttgacttaaagtggctgtgggaagtgttgacgggtggcaaaataaacactaatggtttttggaagaaaccagctttaaacttgatctacaaaccttatcaaatcagaatattagatcctttcattatcagaatggcagcttatgaactgcttcacttcccaaaagtgtttcccaaaaaccagaaacccaaacacccaacaacaggaattattgccatcacgctggcctttcacatatgtcacgaagttcaccttgctggttttaaatacaatttttctgacctcaagagccctttacactattatgggaacgcgaccatgtctttgatgaataagaatgcgtatcacaatgtgacagcggaacagctctttttgaaggacattctagaaaaaaactttgtaatcaacttgactgaagattgaccctacagactctgcagatgatgctaagagtattagttttatttttatactgcaatttttagtttatttttaaatatgttggatgcacttgtcaaaaaattgtgtatagtcagtctgttgctgcctggtgattcataaccaccagcttaatttctgtgaatatatttaatttataaaaaccaagaagatatgcttagatatccgggaagttttgattgcgttggttttaaaacaaccttagttctctgaagtgtttttaaacatcttttttaatagttacttcatctttgacttctgagggcatgtgacgtccaagtaaggggctttagcttgaccaccacaaactctgaacagagttggtggcggattcggctactgtaaattggtggggaatagccatgtgattgtgcaaactggaaccggtttaggcaagtatcgagttcctttttactgaacccgaggaaacggatttgaatcttaaagcaggcccaaccatagcagtaggtacggttatgaaatctaagatcataatggtttcattaagctttttttcctgtaagtaaaccagattataaaatgaaaggtgtttgtttttaaggtggaggaaacaggctacatgtgaaattctggatgagtaaacaacctaggaatgcaattactaaagtctggtggctgcattattttaaagttcatacaaagaagcagagctaggccacctcaaggagacagttcttaaacgtcatcttttgcctgccttaatatgttaaaatttggaagtttactatttgaaataggaaagatgaatacggcacagtaggtaaatccttcagactcctcaggctgtttttggatttaaatggtcctttcgtgaaaaatctcacttgtccacggtgaaatcccatcttcaaagggaaggcttacccggctacctagggtgcatcagagaagagtcctgctggatgcagacaagtcaaaaccagcctgtccaacaaacgtgcgcccgtctctcttctcaaagagggatggaatgaacagctctcagaagaggtaagagttgaaggacttgttatcctctgagcgataatcgtcatggagagacactgctggtgttcctgaaaaccagcctgcctctgagtctcagagacaaaatatgagagcagccactgggataaatcgtgaagcacggcataaggggggagaagcctcgtagttgattgaacccatgtctacgtggcttcagctgattcccctgtaacggaggtggaaagttcccgcacgtacacagctgcacgctgcagcctggcggctgggattccatgggtggactcattcagggtacaaagacagtcctggctgcaaagtgaaaaaccccaggtggcattttcaagtgtttatggactgaaataatggctgtacggtatctggcggatgctcaacttgaggaatcggcatttttgtacagtgggagctgaggctataaacctcagcgtggcttcacataagccagaagaaactctcagcccgatacatatgtacaatttattaaaaacacatgaacacgttaaaatctcactatttatacaatctacattctagcaacatatacaaataccgagtgactacagtacatgccgaggtaagaaaagtacattcggggagactatcactgacactcaagccatttttatttccaatatgttttgctttcacctttcccagtgccaaaaaaaaaaaaaaaaaa

SEQ ID NO: 16 - protein, 331 aa, Canis lupus
MRGYLVAIFLSAVFLYYVLHCILWGTNIYWVPPVEMKRRNKIQPCLAKPAFASLLRFHQFHPFLCAADFKKIASLYGSDKFDLPYGIRTSAEYFRLALSKLQSCDLFDEFDNVPCKKCVVVGNGGVLKNKTLGEKIDSYDVIIRMNNGPVLGHEEEVGRRTTFRLFYPESVFSDPNHNDPNTTAILTAFKPLDLKWLWEVLTGGKINTNGFWKKPALNLIYKPYQIRILDPFIIRMAAYELLHFPKVFPKNQKPKHPTTGIIAITLAFHICHEVHLAGFKYNFSDLKSPLHYYGNATMSLMNKNAYHNVTAEQLFLKDILEKNFVINLTED

SEQ ID NO: 17 - DNA, 1337 bp, Canis lupus
  source 1..1337: mol_type = other DNA
aaagacttcactgggtatcagtctcctttgggagaccacaggacacgtgtcacctctcccatcctctcagcctccagcccagaccttggcagagttccttttaggagttagcaagtggctgaggaggcaagaggtgccagagccaatctactatctgctgggggatgattgccagggccagagatgagggctcaatacttgaagtggggtctggtagctgcctgtatagttacgttatggctgatgatgatgaacttcctggaccaggagttcaaacagaatgacttccctaaaaagacaagaatacaattatgccactgccccaggaactctttcagaaagtgtaggtgttcgtttgagatccgcaagtgctctgcctgcctccgcgtacgtggaacgtctgtctggtttgatgaacgcttcgaaacggctattgagcctgtgcagagaccagaagatcccatatcctctgatgctctgatattgtggttgggtgtccaatcaaagagggagtttgagactcagaagccaatagaagagcctcctgggcaaccactgggctacgtggagtccagttgtcggacctgtgcagtggttggaaactcaaggtgcctacggggctctggccatggattcaggattaaccaaaatgacatggtcctcaggatgaaccaggcccccgtccaaggatttgagatggatgtggggaacacaaccaccatgcgcataatgtaccccgatatggctagcacgcagaatcctggcaccaaattgctgctgcttcctctgaattcatctggtctaaagtggtttatggaagtactacaggaacagagcttcagaaagcccataaaccctggatttcagatagtccagtttcctggtggaagtaacacgagcaaagacgaggtcttggtgatcagcctcacctttcttcagtacatccaggatcattggctgcgaaaacgtcatcgttttccatccttggggtttgtgggtctgttatatgccctgcacacttgtgaccaggtatccttatttggttttgggacagatcagctcatgaggtggtcccattactgggatgataaatatcggttcgagagtaacatgcacagtttcaaagaagagcagaagctcatcctccagctgcaatgtgaggggaagattgttatctacagctgacatgtttctgtcctgttcagcccactggaggccccaggaggctgacaggtagtcaaggggaccacagagtgtcagagagggactggggcttcaagtggaccctggatatagatcagtctgctgctaaataaaactacagcttatttctccca

SEQ ID NO: 18 - protein, 333 aa, Canis lupus
MRAQYLKWGLVAACIVTLWLMMMNFLDQEFKQNDFPKKTRIQLCHCPRNSFRKCRCSFEIRKCSACLRVRGTSVWFDERFETAIEPVQRPEDPISSDALILWLGVQSKREFETQKPIEEPPGQPLGYVESSCRTCAVVGNSRCLRGSGHGFRINQNDMVLRMNQAPVQGFEMDVGNTTTMRIMYPDMASTQNPGTKLLLLPLNSSGLKWFMEVLQEQSFRKPINPGFQIVQFPGGSNTSKDEVLVISLTFLQYIQDHWLRKRHRFPSLGFVGLLYALHTCDQVSLFGFGTDQLMRWSHYWDDKYRFESNMHSFKEEQKLILQLQCEGKIVIYS

SEQ ID NO: 19 - skipped

SEQ ID NO: 20 - DNA, 83 bp, synthetic construct
  misc_feature 1..83: note = A synthetic oligonucleotide
  source 1..83: mol_type = other DNA
cctccttcttcctgaattactcccacacccctccttttctgaattactcccacacccctcctttcctgaattactcccacacc

SEQ ID NO: 21 - DNA, 59 bp, synthetic construct
  misc_feature 1..59: note = A synthetic oligonucleotide
  source 1..59: mol_type = other DNA
ctttacctactcccaccacagcatggccactttacctactcccaccacaagcatggcca

SEQ ID NO: 22 - DNA, 87 bp, synthetic construct
  misc_feature 1..87: note = A synthetic oligonucleotide
  source 1..87: mol_type = other DNA
ctcccccgcggctgtggcggccgcccgcgctcccccgcgggctgtggcggccgcccgcgctcccccgcgctgtggcggccgcccgcg

SEQ ID NO: 23 - DNA, 319 bp, synthetic construct
  misc_feature 1..319: note = A synthetic oligonucleotide
  source 1..319: mol_type = other DNA
tccccaggaaccctgctaccctctgagaatccccaggaccctgctaccctctgagaatccccaggacgatggtggtgtcaaagtacttgaaggcggcaggggctcccagattggtcagggtaaacaggtggatgatattctcggcctgctctctgatgggcttatcccggtgcttgttgtaggcggacagcactttgtccagattagcgtcggccaggatcactctcttggagaactcgctgatctgctcgatgatctcgtccaggtagtgcttgtgctgttccacaaacagctgtttcaccctgctaccctctgagaa

SEQ ID NO: 24 - DNA, 135 bp, synthetic construct
  misc_feature 1..135: note = A synthetic oligonucleotide
  source 1..135: mol_type = other DNA
atcgctcaagaccctctctgcaatggtacatcgctcaagaccctctctgatggtacatcgctcaagaccaatggtacatcgctcaagaccctctctggcaatggtacatcgctcaagaccctctctcaatggtac

SEQ ID NO: 25 - DNA, 59 bp, synthetic construct
  misc_feature 1..59: note = A synthetic oligonucleotide
  source 1..59: mol_type = other DNA
agcgataagtttgatctgccctatgggataagcgataagtttgatctgcctatgggata

SEQ ID NO: 26 - DNA, 57 bp, synthetic construct
  misc_feature 1..57: note = A synthetic oligonucleotide
  source 1..57: mol_type = other DNA
gcccccgtccaaggatttgagatggatgtgcccccgtcaaggatttgagatggatgt

SEQ ID NO: 27 - DNA, 20 bp, synthetic construct
  misc_feature 1..20: note = A synthetic oligonucleotide
  source 1..20: mol_type = other DNA
ccctcctcgtcctcttcatc

SEQ ID NO: 28 - DNA, 20 bp, synthetic construct
  misc_feature 1..20: note = A synthetic oligonucleotide
  source 1..20: mol_type = other DNA
aggcagagagagaccagaga

SEQ ID NO: 29 - DNA, 20 bp, synthetic construct
  misc_feature 1..20: note = A synthetic oligonucleotide
  source 1..20: mol_type = other DNA
ccaaaccatgaagtgctccc

SEQ ID NO: 30 - DNA, 20 bp, synthetic construct
  misc_feature 1..20: note = A synthetic oligonucleotide
  source 1..20: mol_type = other DNA
aggggcttgaagagtgactc

SEQ ID NO: 31 - DNA, 20 bp, synthetic construct
  misc_feature 1..20: note = A synthetic oligonucleotide
  source 1..20: mol_type = other DNA
atgagacttgcttgcatccc

SEQ ID NO: 32 - DNA, 22 bp, synthetic construct
  misc_feature 1..22: note = A synthetic oligonucleotide
  source 1..22: mol_type = other DNA
ctttggttggcctctctgtctc

SEQ ID NO: 33 - DNA, 18 bp, synthetic construct
  misc_feature 1..18: note = A synthetic oligonucleotide
  source 1..18: mol_type = other DNA
cgttagccgcgcgcacag

SEQ ID NO: 34 - DNA, 17 bp, synthetic construct
  misc_feature 1..17: note = A synthetic oligonucleotide
  source 1..17: mol_type = other DNA
ccgggatgacagctctc

SEQ ID NO: 35 - DNA, 19 bp, synthetic construct
  misc_feature 1..19: note = A synthetic oligonucleotide
  source 1..19: mol_type = other DNA
acatggaagctggactcac

SEQ ID NO: 36 - DNA, 20 bp, synthetic construct
  misc_feature 1..20: note = A synthetic oligonucleotide
  source 1..20: mol_type = other DNA
catcatcacaaggatcctgc

SEQ ID NO: 37 - DNA, 19 bp, synthetic construct
  misc_feature 1..19: note = A synthetic oligonucleotide
  source 1..19: mol_type = other DNA
ctctcccatgaaaacctgg

SEQ ID NO: 38 - DNA, 21 bp, synthetic construct
  misc_feature 1..21: note = A synthetic oligonucleotide
  source 1..21: mol_type = other DNA
gttttaaatttgggagcggcc

SEQ ID NO: 39 - DNA, 20 bp, synthetic construct
  misc_feature 1..20: note = A synthetic oligonucleotide
  source 1..20: mol_type = other DNA
tggctcacatcaaacaccac

SEQ ID NO: 40 - DNA, 20 bp, synthetic construct
  misc_feature 1..20: note = A synthetic oligonucleotide
  source 1..20: mol_type = other DNA
ggttggaaactcaaggtgcc

SEQ ID NO: 41 - DNA, 20 bp, synthetic construct
  misc_feature 1..20: note = A synthetic oligonucleotide
  source 1..20: mol_type = other DNA
tgactccttccccttttccc

SEQ ID NO: 42 - DNA, 37 bp, synthetic construct
  misc_feature 1..37: note = A synthetic oligonucleotide
  source 1..37: mol_type = other DNA
gttacgcgccagcaaaagcaggggaaaacaaaagcaa

SEQ ID NO: 43 - DNA, 38 bp, synthetic construct
  misc_feature 1..38: note = A synthetic oligonucleotide
  source 1..38: mol_type = other DNA
gttacgcgccagtagaaacaagggtgtttttctcatgc

SEQ ID NO: 44 - DNA, 32 bp, synthetic construct
  misc_feature 1..32: note = A synthetic oligonucleotide
  source 1..32: mol_type = other DNA
gttacgcgccagcaaaagcaggagtttaaaat

SEQ ID NO: 45 - DNA, 38 bp, synthetic construct
  misc_feature 1..38: note = A synthetic oligonucleotide
  source 1..38: mol_type = other DNA
gttacgcgccagtagaaacaaggagttttttgaacaac

SEQ ID NO: 46 - DNA, 37 bp, synthetic construct
  misc_feature 1..37: note = A synthetic oligonucleotide
  source 1..37: mol_type = other DNA
gttacgcgccagcaaaagcaggggataattctattaa

SEQ ID NO: 47 - DNA, 39 bp, synthetic construct
  misc_feature 1..39: note = A synthetic oligonucleotide
  source 1..39: mol_type = other DNA
gttacgcgccagtagaaacaagggtgtttttaattaatg

SEQ ID NO: 48 - DNA, 32 bp, synthetic construct
  misc_feature 1..32: note = A synthetic oligonucleotide
  source 1..32: mol_type = other DNA
gttacgcgccagcaaaagcaggagtaaagatg

SEQ ID NO: 49 - DNA, 41 bp, synthetic construct
  misc_feature 1..41: note = A synthetic oligonucleotide
  source 1..41: mol_type = other DNA
gttacgcgccagtagaaacaaggagttttttctaaaattgc

SEQ ID NO: 50 - DNA, 38 bp, synthetic construct
  misc_feature 1..38: note = A synthetic oligonucleotide
  source 1..38: mol_type = other DNA
gttacgcgccagcagaagcagagcattttctaatatcc

SEQ ID NO: 51 - DNA, 43 bp, synthetic construct
  misc_feature 1..43: note = A synthetic oligonucleotide
  source 1..43: mol_type = other DNA
gttacgcgccagtagtaacaagagcatttttcaataacgtttc

SEQ ID NO: 52 - DNA, 39 bp, synthetic construct
  misc_feature 1..39: note = A synthetic oligonucleotide
  source 1..39: mol_type = other DNA
gttacgcgccagcagaagcagagcatcttctcaaaactg

SEQ ID NO: 53 - DNA, 38 bp, synthetic construct
  misc_feature 1..38: note = A synthetic oligonucleotide
  source 1..38: mol_type = other DNA
gttacgcgccagtagtaacaagagcatttttcagaaac

SEQ ID NO: 54 - DNA, 30 bp, synthetic construct
  misc_feature 1..30: note = A synthetic oligonucleotide
  source 1..30: mol_type = other DNA
agaaaagaatgtaacagtaacacactctgt

SEQ ID NO: 55 - DNA, 22 bp, synthetic construct
  misc_feature 1..22: note = A synthetic oligonucleotide
  source 1..22: mol_type = other DNA
tgtttccacaatgtargaccat

SEQ ID NO: 56 - DNA, 27 bp, synthetic construct
  misc_feature 1..27: note = A synthetic oligonucleotide
  source 1..27: mol_type = other DNA
ctattggacaatagtaaaaccgggrga

SEQ ID NO: 57 - DNA, 24 bp, synthetic construct
  misc_feature 1..24: note = A synthetic oligonucleotide
  source 1..24: mol_type = other DNA
gtcattgggratgcttccatttgg

SEQ ID NO: 58 - skipped

SEQ ID NO: 59 - DNA, 30 bp, synthetic construct
  misc_feature 1..30: note = A synthetic oligonucleotide
  source 1..30: mol_type = other DNA
cctgttacatctgggtgctttcctataatg

SEQ ID NO: 60 - DNA, 30 bp, synthetic construct
  misc_feature 1..30: note = A synthetic oligonucleotide
  source 1..30: mol_type = other DNA
gttgatarcctgatatgttcgtatcctckg

SEQ ID NO: 61 - DNA, 30 bp, synthetic construct
  misc_feature 1..30: note = A synthetic oligonucleotide
  source 1..30: mol_type = other DNA
cctgttacatccgggtgcttycctataatg

SEQ ID NO: 62 - DNA, 30 bp, synthetic construct
  misc_feature 1..30: note = A synthetic oligonucleotide
  source 1..30: mol_type = other DNA
gttgataacctkatmttttcatatcctctg

SEQ ID NO: 63 - DNA, 29 bp, synthetic construct
  misc_feature 1..29: note = A synthetic oligonucleotide
  source 1..29: mol_type = other DNA
ccmaggtcgaaacgtaygttctctctatc

SEQ ID NO: 64 - DNA, 32 bp, synthetic construct
  misc_feature 1..32: note = A synthetic oligonucleotide
  source 1..32: mol_type = other DNA
tgacagratyggtcttgtctttagccaytcca

SEQ ID NO: 65 - DNA, 17 bp, synthetic construct
  misc_feature 1..17: note = A synthetic oligonucleotide
  source 1..17: mol_type = other DNA
ggagcaaccaatgccac

SEQ ID NO: 66 - DNA, 20 bp, synthetic construct
  misc_feature 1..20: note = A synthetic oligonucleotide
  source 1..20: mol_type = other DNA
gtktaggcggtcttgaccag

SEQ ID NO: 67 - DNA, 23 bp, synthetic construct
  misc_feature 1..23: note = A synthetic oligonucleotide
  source 1..23: mol_type = other DNA
cagccagcaatrttrcatttacc

SEQ ID NO: 68 - DNA, 23 bp, synthetic construct
  misc_feature 1..23: note = A synthetic oligonucleotide
  source 1..23: mol_type = other DNA
cagccagcaatrttrcatttacc

SEQ ID NO: 69 - DNA, 23 bp, synthetic construct
  misc_feature 1..23: note = A synthetic oligonucleotide
  source 1..23: mol_type = other DNA
aagtaacccckaggagcaattag

SEQ ID NO: 70 - DNA, 23 bp, synthetic construct
  misc_feature 1..23: note = A synthetic oligonucleotide
  source 1..23: mol_type = other DNA
aagtaacccckaggagcaattag

SEQ ID NO: 71 - DNA, 18 bp, synthetic construct
  misc_feature 1..18: note = A synthetic oligonucleotide
  source 1..18: mol_type = other DNA
ttagacagctgcctaacc

SEQ ID NO: 72 - DNA, 18 bp, synthetic construct
  misc_feature 1..18: note = A synthetic oligonucleotide
  source 1..18: mol_type = other DNA
ttagacagctgcctaacc

SEQ ID NO: 73 - DNA, 18 bp, synthetic construct
  misc_feature 1..18: note = A synthetic oligonucleotide
  source 1..18: mol_type = other DNA
tcaggcaactasccaatc

SEQ ID NO: 74 - DNA, 18 bp, synthetic construct
  misc_feature 1..18: note = A synthetic oligonucleotide
  source 1..18: mol_type = other DNA
tcaggcaactasccaatc

SEQ ID NO: 75 - DNA, 22 bp, synthetic construct
  misc_feature 1..22: note = A synthetic oligonucleotide
  source 1..22: mol_type = other DNA
atytcggctttgagggggcctg

SEQ ID NO: 76 - DNA, 20 bp, synthetic construct
  misc_feature 1..20: note = A synthetic oligonucleotide
  source 1..20: mol_type = other DNA
ataaactttgaagcaggaat

SEQ ID NO: 77-99 - skipped (23 entries)

SEQ ID NO: 100 - DNA, 1221 bp, Homo sapiens
  source 1..1221: mol_type = other DNA
atgattcacaccaacctgaagaaaaagttcagctgctgcgtcctggtctttcttctgtttgcagtcatctgtgtgtggaaggagaagaagaaagggagttactatgattcctttaaattgcaaaccaaggaattccaggtgttaaagagtctggggaaattggccatggggtctgattcccagtctgtatcctcaagcagcacccaggacccccacaggggccgccagaccctcggcagtctcagaggcctagccaaggccaaaccagaggcctccttccaggtgtggaacaaggacagctcttccaaaaaccttatccctaggctgcaaaagatctggaagaattacctaagcatgaacaagtacaaagtgtcctacaaggggccaggaccaggcatcaagttcagtgcagaggccctgcgctgccacctccgggaccatgtgaatgtatccatggtagaggtcacagattttcccttcaatacctctgaatgggagggttatctgcccaaggagagcattaggaccaaggctgggccttggggcaggtgtgctgttgtgtcgtcagcgggatctctgaagtcctcccaactaggcagagaaatcgatgatcatgacgcagtcctgaggtttaatggggcacccacagccaacttccaacaagatgtgggcacaaaaactaccattcgcctgatgaactctcagttggttaccacagagaagcgcttcctcaaagacagtttgtacaatgaaggaatcctaattgtatgggacccatctgtataccacccagatatcccaaagtggtaccagaatccggattataatttctttaacaactacaagacttatcgtaagctgcaccccaatcagcccttttacatcctcaagccccagatgccttgggagctatgggacattcttcaagaaatctccccagaagagattcagccaaaccccccatcctctgggatgcttggtatcatcatcatgatgacgctgtgtgaccaggtggatatttatgagttcctcccatccaagcgcaggactgacgtgtgctactactaccagaagttcttcgatagtgcctgcacgatgggtgcctaccacccgctgctctttgagaagaatttggtgaagcatctcaaccagggcacagatgaggacatctacctgcttggaaaagccacactgcctggcttccggaccattcactgctaa

SEQ ID NO: 101 - protein, 406 aa, Homo sapiens
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHPDIPKWYQNPDYNFFNNYKTYRKLHPNQPFYILKPQMPWELWDILQEISPEEIQPNPPSSGMLGIIIMMTLCDQVDIYEFLPSKRRTDVCYYYQKFFDSACTMGAYHPLLFEKNLVKHLNQGTDEDIYLLGKATLPGFRTIHC

SEQ ID NO: 102-149 - skipped (48 entries)

SEQ ID NO: 150 - protein, 406 aa, Homo sapiens; identical to SEQ ID NO: 101

SEQ ID NO: 151 - DNA, 1221 bp, Homo sapiens; identical to SEQ ID NO: 100
  source 1..1221: mol_type = other DNA